SEQUENCE LISTING (102 sequences)

SEQ ID NO: 1 - DNA, 1662 bp, Cypridina noctiluca
  CDS (1)..(1662)
atgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggctgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcatagaattcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctcgagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagatatcaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaatag

SEQ ID NO: 2 - protein, 553 aa, Cypridina noctiluca
MKTLILAVALVYCATVHCQDCPYEPDPPNTVPTSCEAKEGECIDSSCGTCTRDILSDGLCENKPGKTCCRMCQYVIECRVEAAGWFRTFYGKRFQFQEPGTYVLGQGTKGGDWKVSITLENLDGTKGAVLTKTRLEVAGDIIDIAQATENPITVNGGADPIIANPYTIGEVTIAVVEMPGFNITVIEFFKLIVIDILGGRSVRIAPDTANKGMISGLCGDLKMMEDTDFTSDPEQLAIQPKINQEFDGCPLYGNPDDVAYCKGLLEPYKDSCRNPINFYYYTISCAFARCMGGDERASHVLLDYRETCAAPETRGTCVLSGHTFYDTFDKARYQFQGPCKEILMAADCFWNTWDVKVSHRNVDSYTEVEKVRIRKQSTVVELIVDGKQILVGGEAVSVPYSSQNTSIYWQDGDILTTAILPEALVVKFNFKQLLVVHIRDPFDGKTCGICGNYNQDFSDDSFDAEGACDLTPNPPGCTEEQKPEAERLCNSLFAGQSDLDQKCNVCHKPDRVERCMYEYCLRGQQGFCDHAWEFKKECYIKHGDTLEVPDECK

SEQ ID NO: 3 - DNA, 1668 bp, Vargula hilgendorfii
  CDS (1)..(1668)
atgaagataataattctgtctgttatattggcctactgtgtcaccgacaactgtcaagatgcatgtcctgtagaagcggaaccgccatcaagtacaccaacagttccaacttcttgtgaagctaaagaaggagaatgtatagataccagatgcgcaacatgtaaacgagatatactatcagatggactgtgtgaaaataaaccagggaagacatgctgtagaatgtgccagtatgtgattgaatgcagagtagaagcagctggttattttagaacgttttacggcaaaagatttaattttcaggaacctggtaaatatgtgctggctaggggaaccaagggtggcgattggtctgtaaccctcaccatggagaatctagatggacagaagggagctgtgctgactaagacaacactggaggttgcaggagacgtaatagacattactcaagctactgcagatcctatcacagttaacggaggagctgacccagttatcgctaacccgttcacaattggtgaggtgaccattgctgttgttgaaataccgggcttcaatatcacagtcatcgaattctttaaactaatcgtgattgatattctgggaggaagatctgtgagaattgctccagacacagcaaacaaaggactgatatctggtatctgtggtaatctggagatgaatgacgctgatgactttactacagatgcagatcagctggcgatccaacccaacataaacaaagagttcgacggctgcccattctatggcaatccttctgatatcgaatactgcaaaggtctgatggagccatacagagctgtatgtcgtaacaatatcaacttctactattacactctatcctgtgccttcgcttactgtatgggaggagaagaaagagctaaacacgtccttttcgactatgttgagacatgcgctgcgccggaaacgagaggaacgtgtgttttatcaggacatactttctatgacacattcgacaaagcaagatatcaattccagggcccatgcaaggagattctgatggccgcagactgttactggaacacatgggatgtaaaggtttcacatagagacgtcgaatcatacactgaggtagagaaagtaacaatcaggaaacagtcaactgtagtagatctcattgtggatggcaagcaggtcaaggttggaggagtggatgtatctatcccgtacagctctgagaacacttccatatactggcaggatggagacatcctgacgacggccatcctacctgaagctcttgtcgttaagttcaactttaagcagctccttgtagttcatatcagagatccattcgatggaaagacatgcggcatatgtggtaactataatcaagattcaactgatgatttctttgacgcagaaggagcatgcgctctaacccccaaccccccaggatgtacagaggaacagaaaccagaagctgagcgactttgcaataatctctttgattcttctatcgacgagaaatgtaatgtctgctacaagcctgaccggattgcccgatgtatgtacgagtattgcctgaggggacaacaaggattttgtgaccatgcttgggagttcaagaaagaatgctacataaaacatggagacactctagaagtaccacctgaatgtcaataa

SEQ ID NO: 4 - protein, 555 aa, Vargula hilgendorfii
MKIIILSVILAYCVTDNCQDACPVEAEPPSSTPTVPTSCEAKEGECIDTRCATCKRDILSDGLCENKPGKTCCRMCQYVIECRVEAAGYFRTFYGKRFNFQEPGKYVLARGTKGGDWSVTLTMENLDGQKGAVLTKTTLEVAGDVIDITQATADPITVNGGADPVIANPFTIGEVTIAVVEIPGFNITVIEFFKLIVIDILGGRSVRIAPDTANKGLISGICGNLEMNDADDFTTDADQLAIQPNINKEFDGCPFYGNPSDIEYCKGLMEPYRAVCRNNINFYYYTLSCAFAYCMGGEERAKHVLFDYVETCAAPETRGTCVLSGHTFYDTFDKARYQFQGPCKEILMAADCYWNTWDVKVSHRDVESYTEVEKVTIRKQSTVVDLIVDGKQVKVGGVDVSIPYSSENTSIYWQDGDILTTAILPEALVVKFNFKQLLVVHIRDPFDGKTCGICGNYNQDSTDDFFDAEGACALTPNPPGCTEEQKPEAERLCNNLFDSSIDEKCNVCYKPDRIARCMYEYCLRGQQGFCDHAWEFKKECYIKHGDTLEVPPECQ

SEQ ID NO: 5 - DNA, 591 bp, Oplophorus gracilirostris
  CDS (1)..(591)
atggcgtactccactctgttcataattgcattgaccgccgttgtcactcaagcttcctcaactcaaaaatctaacctaacttttacgttggcagatttcgttggagactggcaacagacagctggatacaaccaagatcaagtgttagaacaaggaggattgtctagtctgttccaagccctgggagtgtcagtcacgcccatacagaaagttgtactgtctggggagaatgggttaaaagctgatattcatgtcataataccttacgagggactcagtggttttcaaatgggtctaattgaaatgatcttcaaagttgtttaccccgtggatgatcatcatttcaagattattctccattatggtacactcgttattgacggtgtaacacccaacatgattgactactttggaagaccttaccctggaattgctgtatttgacggcaagcagatcacagttactggaactctgtggaacggcaacaagatctatgatgagaggctaatcaaccctgatggttcactcctcttcagagttactatcaatggagtcacgggatggaggctttgcgagaacattcttgcctaa

SEQ ID NO: 6 - protein, 196 aa, Oplophorus gracilirostris
MAYSTLFIIALTAVVTQASSTQKSNLTFTLADFVGDWQQTAGYNQDQVLEQGGLSSLFQALGVSVTPIQKVVLSGENGLKADIHVIIPYEGLSGFQMGLIEMIFKVVYPVDDHHFKIILHYGTLVIDGVTPNMIDYFGRPYPGIAVFDGKQITVTGTLWNGNKIYDERLINPDGSLLFRVTINGVTGWRLCENILA

SEQ ID NO: 7 - DNA, 660 bp, Metridia longa
  CDS (1)..(660)
atggatataaaggttgtctttactcttgttttctcagcattggttcaggcaaaatcaactgaattcgatcctaacattgacattgttggtttagaaggaaaatttggtataacaaaccttgagacggatttattcacaatatgggagacaatggaggtcatgatcaaagcagatattgcagatactgatagagccagcaactttgttgcaactgaaaccgatgctaaccgtggaaaaatgcctggcaaaaaactgccactggcagttatcatggaaatggaagccaatgctttcaaagctggctgcaccaggggatgccttatctgtctttcaaaaataaagtgtacagccaaaatgaaggtgtacattccaggaagatgtcatgattatggtggtgacaagaaaactggacaggcaggaatagttggtgcaattgttgacattcccgaaatctctggatttaaggagatggcacccatggaacagttcattgctcaagttgatcgttgcgcttcctgcactactggatgtctcaaaggtcttgccaatgttaagtgctctgaactcctgaagaaatggctgcctgacagatgtgcaagttttgctgacaagattcaaaaagaagttcacaatatcaaaggcatggctggagatcgttga

SEQ ID NO: 8 - protein, 219 aa, Metridia longa
MDIKVVFTLVFSALVQAKSTEFDPNIDIVGLEGKFGITNLETDLFTIWETMEVMIKADIADTDRASNFVATETDANRGKMPGKKLPLAVIMEMEANAFKAGCTRGCLICLSKIKCTAKMKVYIPGRCHDYGGDKKTGQAGIVGAIVDIPEISGFKEMAPMEQFIAQVDRCASCTTGCLKGLANVKCSELLKKWLPDRCASFADKIQKEVHNIKGMAGDR

SEQ ID NO: 9 - DNA, 1560 bp, Homo sapiens
  CDS (1)..(1560)
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatga

SEQ ID NO: 10 - protein, 519 aa, Homo sapiens
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYSRVGAAGRFEQT

SEQ ID NO: 11 - DNA, 267 bp, Saccharomyces cerevisiae
  CDS (1)..(267)
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 12 - protein, 89 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 13 - DNA, 1875 bp, Artificial Sequence
  Description of Artificial Sequence a gene encoding a fusion protein
  CDS (1)..(1875)
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctcaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggctgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcatagaattcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctcgagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagatatcaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaatag

SEQ ID NO: 14 - protein, 624 aa, Artificial Sequence
  Description of Artificial Sequence fusion protein
MRFPSIFTAVLFAASSALAAPVNTTTEDETAQIPAEAVIGYSDLEGDFDVAVLPFSNSTNNGLLFINTTIASIAAKEEGVSLEKREAEAQDCPYEPDPPNTVPTSCEAKEGECIDSSCGTCTRDILSDGLCENKPGKTCCRMCQYVIECRVEAAGWFRTFYGKRFQFQEPGTYVLGQGTKGGDWKVSITLENLDGTKGAVLTKTRLEVAGDIIDIAQATENPITVNGGADPIIANPYTIGEVTIAVVEMPGFNITVIEFFKLIVIDILGGRSVRIAPDTANKGMISGLCGDLKMMEDTDFTSDPEQLAIQPKINQEFDGCPLYGNPDDVAYCKGLLEPYKDSCRNPINFYYYTISCAFARCMGGDERASHVLLDYRETCAAPETRGTCVLSGHTFYDTFDKARYQFQGPCKEILMAADCFWNTWDVKVSHRNVDSYTEVEKVRIRKQSTVVELIVDGKQILVGGEAVSVPYSSQNTSIYWQDGDILTTAILPEALVVKFNFKQLLVVHIRDPFDGKTCGICGNYNQDFSDDSFDAEGACDLTPNPPGCTEEQKPEAERLCNSLFAGQSDLDQKCNVCHKPDRVERCMYEYCLRGQQGFCDHAWEFKKECYIKHGDTLEVPDECK

SEQ ID NO: 15 - DNA, 1662 bp, Artificial Sequence
  Description of Artificial Sequence a synthesized gene optimized for use in Saccharomyces cerevisiae
atgaagaccttgatcttggctgtcgctttggtctactgtgctactgttcactgtcaagactgtccatacgaaccagacccaccgaacactgtccctacctcttgtgaagctaaagaaggtgaatgtatcgattcttcttgtggtacttgtaccagggacatattgtctgacggtttgtgtgaaaacaagcctggcaagacttgttgtagaatgtgccaatacgttatcgaatgcagagttgaagcggcagggtggttcaggactttttacggtaaacgtttccaattccaagaaccaggtacttacgtcttaggccaaggtaccaaaggtggagattggaaagtatccataacattagagaatcttgatggcactaagggtgctgtcttaactaaaacccggttagaagtcgctggtgatattatcgacatcgctcaagctactgagaatccgattactgttaatggtggtgctgatcctatcatcgccaacccttatactatcggtgaggttaccatcgccgtcgttgagatgccaggctttaacattaccgtaatcgagttcttcaagttgatcgtcatcgacatcttgggtggtcgttctgtcagaatcgctccagacactgctaacaagggtatgatctctggtttgtgtggcgatctcaagatgatggaggacactgatttcacttcagatcctgagcagctagctatccaacctaagattaaccaagagttcgatgggtgtccgttatacggtaatcctgatgacgtcgcttactgtaaaggcttgttggaaccatacaaagattcttgtaggaatcctatcaacttttactactataccatatcttgtgctttcgccaggtgcatggggggagacgagcgtgcatctcatgttttgttggactacagagagacttgtgctgctccagaaactagaggtacttgtgtcttgtctggtcacactttctacgacactttcgacaaggctaggtaccaattccaagggccgtgcaaggaaatactaatggctgcggactgcttctggaacacttgggacgtcaaggtctctcaccgtaacgtggattcttacactgaagtcgaaaaggtcaggattcgtaagcagtccactgtcgtcgaattgatcgttgacggtaagcaaatcttggtcggtggtgaagccgtttctgtgccatattcctctcaaaacacttcaatatactggcaagacggtgacatcttaaccactgctatcttgccagaagccttggtcgtcaagttcaacttcaagcagctattagttgttcatattagagacccattcgacggtaagacttgtggtatctgtggtaactacaatcaggacttctctgacgattctttcgacgctgaaggtgcttgcgatctaacaccgaatccacctggttgtactgaagaacaaaagccagaagctgaacgtttgtgcaacagtttattcgctggtcaatccgatttggaccaaaagtgtaacgtctgtcacaagccagacagagttgaaagatgtatgtacgaatactgtttgagaggtcaacaaggtttctgtgaccatgcttgggagttcaagaaggagtgctatatcaagcacggcgataccttagaggtcccagatgagtgcaagtag

SEQ ID NO: 16 - DNA, 39 bp, Artificial Sequence
  Description of Artificial Sequence primer
gaaaagagaggctgaagctcaggactgtccttacgaacc

SEQ ID NO: 17 - DNA, 32 bp, Artificial Sequence
  Description of Artificial Sequence primer
ccctgtctagactatttgcattcatctggtac

SEQ ID NO: 18 - DNA, 29 bp, Artificial Sequence
  Description of Artificial Sequence primer
gggtcccgggatgagatttccttcaattt

SEQ ID NO: 19 - DNA, 39 bp, Artificial Sequence
  Description of Artificial Sequence primer
ggttcgtaaggacagtcctgagcttcagcctctcttttc

SEQ ID NO: 20 - DNA, 36 bp, Artificial Sequence
  Description of Artificial Sequence Oligo DNA linker
ccgctcgagcggccgcgagctcgtcgacatcgatgg

SEQ ID NO: 21 - DNA, 36 bp, Artificial Sequence
  Description of Artificial Sequence Oligo DNA linker
ccatcgatgtcgacgagctcgcggccgctcgagcgg

SEQ ID NO: 22 - DNA, 653 bp, Saccharomyces cerevisiae
tcgagtttatcattatcaatactcgccatttcaaagaatacgtaaataattaatagtagtgattttcctaactttatttagtcaaaaaattagccttttaattctgctgtaacccgtacatgccaaaatagggggcgggttacacagaatatataacactgatggtgcttgggtgaacaggtttattcctggcatccactaaatataatggagcccgctttttaagctggcatccagaaaaaaaaagaatcccagcaccaaaatattgttttcttcaccaaccatcagttcataggtccattctcttagcgcaactacagagaacagggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggtttaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcga

SEQ ID NO: 23 - DNA, 31 bp, Artificial Sequence
  Description of Artificial Sequence primer
gggtggatcccgagtttatcattatcaatac

SEQ ID NO: 24 - DNA, 20 bp, Artificial Sequence
  Description of Artificial Sequence primer
tcgaaactaagttcttggtg

SEQ ID NO: 25 - DNA, 70 bp, Artificial Sequence
  Description of Artificial Sequence synthesized DNA encoding portion of a luciferase
catgaagaccttgatcttggctgtcgctttggtctactgtgctactgttcactgtcaagactgtccatac

SEQ ID NO: 26 - DNA, 70 bp, Artificial Sequence
  Description of Artificial Sequence synthesized DNA encoding portion of a luciferase
gatacattcaccttctttagcttcacaagaggtagggacagtgttcggtgggtctggttcgtatggacag

SEQ ID NO: 27 - DNA, 70 bp, Artificial Sequence
  Description of Artificial Sequence synthesized DNA encoding portion of a luciferase
tgaatgtatcgattcttcttgtggtacttgtaccagggacatattgtctgacggtttgtgtgaaaacaag

SEQ ID NO: 28 - DNA, 70 bp, Artificial Sequence
  Description of Artificial Sequence synthesized DNA encoding portion of a luciferase
tgccgcttcaactctgcattcgataacgtattggcacattctacaacaagtcttgccaggcttgttttca

SEQ ID NO: 29 - DNA, 70 bp, Artificial Sequence
  Description of Artificial Sequence synthesized DNA encoding portion of a luciferase
tgaagcggcagggtggttcaggactttttacggtaaacgtttccaattccaagaaccaggtacttacgtc

SEQ ID NO: 30 - DNA, 70 bp, Artificial Sequence
  Description of Artificial Sequence synthesized DNA encoding portion of a luciferase
atcaagattctctaatgttatggatactttccaatctccacctttggtaccttggcctaagacgtaagta

SEQ ID NO: 31 - DNA, 70 bp, Artificial Sequence
  Description of Artificial Sequence synthesized DNA encoding portion of a luciferase
gaatcttgatggcactaagggtgctgtcttaactaaaacccggttagaagtcgctggtgatattatcgac

SEQ ID NO: 32 - DNA, 70 bp, Artificial Sequence
  Description of Artificial Sequence synthesized DNA encoding portion of a luciferase
ggcgatgataggatcagcaccaccattaacagtaatcggattctcagtagcttgagcgatgtcgataata

SEQ ID NO: 33 - DNA, 70 bp, Artificial Sequence
  Description of Artificial Sequence synthesized DNA encoding portion of a luciferase
tatcatcgccaacccttatactatcggtgaggttaccatcgccgtcgttgagatgccaggctttaacatt

SEQ ID NO: 34 - DNA, 70 bp, Artificial Sequence
  Description of Artificial Sequence synthesized DNA encoding portion of a luciferase
tctgacagaacgaccacccaagatgtcgatgacgatcaacttgaagaactcgattacggtaatgttaaag

SEQ ID NO: 35 - DNA, 70 bp, Artificial Sequence
  Description of Artificial Sequence synthesized DNA encoding portion of a luciferase
ttctgtcagaatcgctccagacactgctaacaagggtatgatctctggtttgtgtggcgatctcaagatg

SEQ ID NO: 36 - DNA, 70 bp, Artificial Sequence
  Description of Artificial Sequence synthesized DNA encoding portion of a luciferase
gttaatcttaggttggatagctagctgctcaggatctgaagtgaaatcagtgtcctccatcatcttgaga

SEQ ID NO: 37 - DNA, 70 bp, Artificial Sequence
  Description of Artificial Sequence synthesized DNA encoding portion of a luciferase
taagattaaccaagagttcgatgggtgtccgttatacggtaatcctgatgacgtcgcttactgtaaaggc

SEQ ID NO: 38 - DNA, 70 bp, Artificial Sequence
  Description of Artificial Sequence synthesized DNA encoding portion of a luciferase
tatggtatagtagtaaaagttgataggattcctacaagaatctttgtatggttccaacaagcctttacag

SEQ ID NO: 39 - DNA, 70 bp, Artificial Sequence
  Description of Artificial Sequence synthesized DNA encoding portion of a luciferase
ctataccatatcttgtgctttcgccaggtgcatggggggagacgagcgtgcatctcatgttttgttggac

SEQ ID NO: 40 - DNA, 70 bp, Artificial Sequence
  Description of Artificial Sequence synthesized DNA encoding portion of a luciferase
agtgtgaccagacaagacacaagtacctctagtttctggagcagcacaagtctctctgtagtccaacaaa

SEQ ID NO: 41 - DNA, 70 bp, Artificial Sequence
  Description of Artificial Sequence synthesized DNA encoding portion of a luciferase
tggtcacactttctacgacactttcgacaaggctaggtaccaattccaagggccgtgcaaggaaatacta

SEQ ID NO: 42 - DNA, 70 bp, Artificial Sequence
  Description of Artificial Sequence synthesized DNA encoding portion of a luciferase
atccacgttacggtgagagaccttgacgtcccaagtgttccagaagcagtccgcagccattagtatttcc

SEQ ID NO: 43 - DNA, 70 bp, Artificial Sequence
  Description of Artificial Sequence synthesized DNA encoding portion of a luciferase
taacgtggattcttacactgaagtcgaaaaggtcaggattcgtaagcagtccactgtcgtcgaattgatc

SEQ ID NO: 44 - DNA, 70 bp, Artificial Sequence
  Description of Artificial Sequence synthesized DNA encoding portion of a luciferase
ttgagaggaatatggcacagaaacggcttcaccaccgaccaagatttgcttaccgtcaacgatcaattcg

SEQ ID NO: 45 - DNA, 70 bp, Artificial Sequence
  Description of Artificial Sequence synthesized DNA encoding portion of a luciferase
ttcctctcaaaacacttcaatatactggcaagacggtgacatcttaaccactgctatcttgccagaagcc

SEQ ID NO: 46 - DNA, 70 bp, Artificial Sequence
  Description of Artificial Sequence synthesized DNA encoding portion of a luciferase
gtcgaatgggtctctaatatgaacaactaatagctgcttgaagttgaacttgacgaccaaggcttctggc

SEQ ID NO: 47 - DNA, 70 bp, Artificial Sequence
  Description of Artificial Sequence synthesized DNA encoding portion of a luciferase
cccattcgacggtaagacttgtggtatctgtggtaactacaatcaggacttctctgacgattctttcgac

SEQ ID NO: 48 - DNA, 70 bp, Artificial Sequence
  Description of Artificial Sequence synthesized DNA encoding portion of a luciferase
tggcttttgttcttcagtacaaccaggtggattcggtgttagatcgcaagcaccttcagcgtcgaaagaa

SEQ ID NO: 49 - DNA, 70 bp, Artificial Sequence
  Description of Artificial Sequence synthesized DNA encoding portion of a luciferase
acaaaagccagaagctgaacgtttgtgcaacagtttattcgctggtcaatccgatttggaccaaaagtgt

SEQ ID NO: 50 - DNA, 70 bp, Artificial Sequence
  Description of Artificial Sequence synthesized DNA encoding portion of a luciferase
ccaaaagtgtaacgtctgtcacaagccagacagagttgaaagatgtatgtacgaatactgtttgagaggt

SEQ ID NO: 51 - DNA, 70 bp, Artificial Sequence
  Description of Artificial Sequence synthesized DNA encoding portion of a luciferase
tttgagaggtcaacaaggtttctgtgaccatgcttgggagttcaagaaggagtgctatatcaagcacggc

SEQ ID NO: 52 - DNA, 44 bp, Artificial Sequence
  Description of Artificial Sequence synthesized DNA encoding portion of a luciferase
cctacttgcactcatctgggacctctaaggtatcgccgtgcttg

SEQ ID NO: 53 - DNA, 30 bp, Artificial Sequence
  Description of Artificial Sequence primer
catacccgggatgaagaccttgattttggc

SEQ ID NO: 54 - DNA, 28 bp, Artificial Sequence
  Description of Artificial Sequence primer
ccctgtctagactacttgcactcatctg

SEQ ID NO: 55 - DNA, 29 bp, Artificial Sequence
  Description of Artificial Sequence primer
ccctgtcgacaagcgcgcaattaaccctc

SEQ ID NO: 56 - DNA, 39 bp, Artificial Sequence
  Description of Artificial Sequence primer
ccctgtcgaccctaagcttggcgtaatcatggtcatagc

SEQ ID NO: 57 - DNA, 30 bp, Artificial Sequence
  Description of Artificial Sequence primer
ccctaagcttagacatgataagatacattg

SEQ ID NO: 58 - DNA, 28 bp, Artificial Sequence
  Description of Artificial Sequence primer
cctaccgcggttaccacatttgtagagg

SEQ ID NO: 59 - DNA, 30 bp, Artificial Sequence
  Description of Artificial Sequence primer
cctaccgcggagacatgataagatacattg

SEQ ID NO: 60 - DNA, 28 bp, Artificial Sequence
  Description of Artificial Sequence primer
ccctgtcgacttaccacatttgtagagg

SEQ ID NO: 61 - DNA, 669 bp, Saccharomyces cerevisiae
acaagcgcgcctctaccttgcagacccatataatataataactaaataagtaaataagacacacgcgagaacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttccttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaaca

SEQ ID NO: 62 - DNA, 29 bp, Artificial Sequence
  Description of Artificial Sequence primer
cccattactagtacaagcgcgcctctacc

SEQ ID NO: 63 - DNA, 27 bp, Artificial Sequence
  Description of Artificial Sequence primer
tgttaattcagtaaattttcgatcttg

SEQ ID NO: 64 - DNA, 1078 bp, Saccharomyces cerevisiae
gtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatataca

SEQ ID NO: 65 - DNA, 30 bp, Artificial Sequence
  Description of Artificial Sequence primer
cccaggatccgtatactagaagaatgagcc

SEQ ID NO: 66 - DNA, 25 bp, Artificial Sequence
  Description of Artificial Sequence primer
tgtatatgagatagttgattgtatg

SEQ ID NO: 67 - DNA, 953 bp, Saccharomyces cerevisiae
gttttagtgtgtgaatgaaataggtgtatgttttctttttgctagacaataattaggaacaaggtaagggaactaaagtgtagaataagattaaaaaagaagaacaagttgaaaaggcaagttgaaatttcaagaaaaaagtcaattgaagtacagtaaattgacctgaatatatctgagttccgacaacaatgagtttaccaaagagaacaatggaataggaaactttgaacgaagaaaggaaagcaggaaaggaaaaaatttttaggctcgagaacaatagggcgaaaaaacaggcaacgaacgaacaatggaaaaacgaaaaaaaaaaaaaaaaacacagaaaagaatgcagaaagatgtcaactgaaaaaaaaaaaggtgaacacaggaaaaaaaataaaaaaaaaaaaaaaaaaaggaggacgaaacaaaaaagtgaaaaaaaatgaaaatttttttggaaaaccaagaaatgaattatatttccgtgtgagacgacatcgtcgaatatgattcagggtaacagtattgatgtaatcaatttcctacctgaatctaaaattcccgggagcaagatcaagatgttttcaccgatctttccggtctctttggccggggtttacggacgatggcagaagaccaaagcgccagttcatttggcgagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaata

SEQ ID NO: 68 - DNA, 30 bp, Artificial Sequence
  Description of Artificial Sequence primer
cccaggatccgttttagtgtgtgaatgaaa

SEQ ID NO: 69 - DNA, 25 bp, Artificial Sequence
  Description of Artificial Sequence primer
tattaatttagtgtgtgtatttgtg

SEQ ID NO: 70 - DNA, 579 bp, Saccharomyces cerevisiae
acaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 71 - DNA, 29 bp, Artificial Sequence
  Description of Artificial Sequence primer
cccaggatccacaatgcatactttgtacg

SEQ ID NO: 72 - DNA, 28 bp, Artificial Sequence
  Description of Artificial Sequence primer
tttgtaattaaaacttagattagattgc

SEQ ID NO: 73 - DNA, 464 bp, Saccharomyces cerevisiae
taagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 74 - DNA, 27 bp, Artificial Sequence
  Description of Artificial Sequence primer
cccaggatcctaagccgatcccattac

SEQ ID NO: 75 - DNA, 30 bp, Artificial Sequence
  Description of Artificial Sequence primer
tttatgtgatgattgattgattgattgtac

SEQ ID NO: 76 - DNA, 451 bp, Saccharomyces cerevisiae
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacc

SEQ ID NO: 77 - DNA, 30 bp, Artificial Sequence
  Description of Artificial Sequence primer
cccaactagtacggattagaagccgccgag

SEQ ID NO: 78 - DNA, 24 bp, Artificial Sequence
  Description of Artificial Sequence primer
ggttttttctccttgacgttaaag

SEQ ID NO: 79 - DNA, 610 bp, Saccharomyces cerevisiae
gatcccactaacggcccagccgaaaatggaaaaaaagggtcggtgatgtgtgggtgccagctggcggtagcaatgacgacgtgttgacgggcccttggctcttgggacaaggactagaagccaaaagccagaggcggtaaaaatagcaagactagaatattgctggcatctgttaaggggatatgttgcaacttgcagggggcggcacaaaataacatagaaacgtagtaaagaggggaaaaggaaaaggaaaaggaaaaggaaggaaaaaaacccattgacgtagaaattgaaagaaggaaaggtatacgcaagcattaatacaacccacaaacacagaccagaagcactctagacggagagtaactagatctacagcccctggaaaatcgtttggtcaactttgaggttccggtcgtccccctcttgatctgaaaggtctttctctaaatctatattaaaacgtataaataggacggtgaattgcgttctacttcctcaattgcgtttgatcttatttaatctctctctaatatatagaaaaaaaaaccatctgattattcgataatctcaaacaaacaactcaaaacaaaaaaaactaaatacaaca

SEQ ID NO: 80 - DNA, 29 bp, Artificial Sequence
  Description of Artificial Sequence primer
gggtggatccgatcccactaacggcccag

SEQ ID NO: 81 - DNA, 28 bp, Artificial Sequence
  Description of Artificial Sequence primer
tgttgtatttagttttttttgttttgag

SEQ ID NO: 82 - DNA, 30 bp, Artificial Sequence
  Description of Artificial Sequence primer
gggtcccgggatgaccggttccggagcttg

SEQ ID NO: 83 - DNA, 30 bp, Artificial Sequence
  Description of Artificial Sequence primer
ccctgtctagattacgcgaaatacgggcag

SEQ ID NO: 84 - DNA, 28 bp, Artificial Sequence
  Description of Artificial Sequence primer
atggttagagttgctattaacggtttcg

SEQ ID NO: 85 - DNA, 28 bp, Artificial Sequence
  Description of Artificial Sequence primer
ttaagccttggcaacgtgttcaaccaag

SEQ ID NO: 86 - DNA, 17 bp, Artificial Sequence
  Description of Artificial Sequence primer
gggccgtgcaaggaaat

SEQ ID NO: 87 - DNA, 21 bp, Artificial Sequence
  Description of Artificial Sequence primer
gacgtcccaagtgttccagaa

SEQ ID NO: 88 - DNA, 22 bp, Artificial Sequence
  Description of Artificial Sequence primer
ctgctaaggctgtcggtaaggt

SEQ ID NO: 89 - DNA, 21 bp, Artificial Sequence
  Description of Artificial Sequence primer
tgaaagccataccggtcaact

SEQ ID NO: 90 - DNA, 30 bp, Artificial Sequence
  Description of Artificial Sequence primer
aagaggatccaatggagcccgctttttaag

SEQ ID NO: 91 - DNA, 30 bp, Artificial Sequence
  Description of Artificial Sequence primer
aagaggatccagaatcccagcaccaaaata

SEQ ID NO: 92 - DNA, 30 bp, Artificial Sequence
  Description of Artificial Sequence primer
aagaggatcctgttttcttcaccaaccatc

SEQ ID NO: 93 - DNA, 30 bp, Artificial Sequence
  Description of Artificial Sequence primer
aagaggatccggagtaaatgatgacacaag

SEQ ID NO: 94 - DNA, 30 bp, Artificial Sequence
  Description of Artificial Sequence primer
aagaggatcctgcgtcctcgtcttcaccgg

SEQ ID NO: 95 - DNA, 30 bp, Artificial Sequence
  Description of Artificial Sequence primer
aagaggatccgaaaaattggcagtaacctg

SEQ ID NO: 96 - DNA, 3636 bp, Rattus norvegicus
ggatccgatcgcaaaacaacttgaaagttgtgctcagtaggggtggggatagctccccctcccaagattatacatccggcaaccgccagaaacaggacagtagcatcaacacttgggttcccagggcttgacaatgtatggcttcaaatccattgggagccaccactgaacagctcctgaaggaaaggagcatgtccccagcccggggatggggagagttcaccgtggcagaggaatcactacagaggggccagggctaaaactcagttttcatcccagaagctgagcctcttaacagatagagccccccacctatttccatttaagcctccagcccttttccgtacaacggaatgcaccagaccccgcgggaaaggggagaagcagtactcagtgccccagcaccaaggcctggattattcaatgaggagtcagctcctttttggggggggggggggtcacaaggcatcaaaactccacctctttcctctgccctgctgtgaagggggggggggagagcaacccgcctcgtgacagggggttggcccagcccgccctagccctgaggagggggcggggcagggggagtcctataattggacaggtctgggatccggtcccctgctcagaccccggaggctaaggagttgtttcggaaggagctggtaagacaagcttgggctggcgattcacccagggggcttggtaagacgtgggcaggggaccttgaaatccgctggagtccaggaaacaggcacaattattagaaaagcaggaagcccgatagaagacttagggtggcggggagacaaactaagatcgtgaggtaactagcctttgccggggtcagaacagatggaggagatggaggggtcatctccaggagtatgggactgtcggtgcactgcatacgggctcaactagaaggtagtatcaatctttagcgaaaggacccgcgatgagccttaggttgcgtttatataaatacctactgatttccatcacagtccccaaaattaccctgggccagtttcaaagcgaagatcctcatgcctgagcgtccgaagttctggggtcgcgggagggtaccacttcgcagggatggaggacgattaaaaacttacattagtactaaaccgagagcccggcttagcagggaaggtagaaaggaatgtaaattggaccaccggctgtccctctgcgttgtggaatttgaaccctaggactgggagctggaatttttggcagcgggtccaccccggggtgctgagatagagataggagggggaggtaaatagacctttgggcagatgctctattgtggagatgtttgtgatgactcacagacctgagaagggaattaaggtgggggctctttgggtggcccgagtttctccacccctacccactggtgttcaaagacagcttttccttccgcagactggccaatcacaactgggaagatgaaggctctgtgggccctgctgttggtcccattgctgacaggtatggggcaaggggtggctttccctgctggcaggttggggttggggcagttctgagacctctgagattcaatagccctgagcagctgttttacatgcaggccttgtctttccctatccataatatgagaggattgatgctcagagggtaaaggtgcttgtcataaagctcgaagacttatctcggagcccaacatggtggaaggagagagcggattctctctagttgtcctctgacctccaagggtacacacacagacacacaattaaaaagttgaagtgtgtaatttcttagtcgcttttaaaggggaggtatacattttctaaccccataccagcctagtctcagctctgttctacataggatactagacagtgtatgcttgtagtatctaaacagactccacgactgacttccagacgcactggtgcttcaaagagacccaagagcactgtaggtcctgacccagccttaaacttactactctacacaggatgcctggccgagggagagctggaggtgacagatcagctcccagggcaaagcgaccaaccctgggagcaggccctgaaccgcttctgggattacctgcgctgggtgcagacgctttctgaccaggtccaggaagagctgcagagctcccaagtcacacaggaactgacgtgagtgctcagcgcttcaccctccgcacctggtgagtatccagatccaggggttcctcctatctgggcacctacctacttgtttcctttctccatgagtgtgtgggccaggttggccttgaactctcaatacttctgctttctttagccttctggatactgggatgaacaggcattcatttatgttcttggtcgaatggcttttggctttttgagacaggatcccaatctaacttaagctggcttcgaaggctctgcaattctccttcctcagcttctcaacttctgggaatacaagcgagtaccaccgcacctcgctctgtggtttcctcccaccctaattctgacctttttgtctctgcattcatctccctcttgtgtttcctctgggcctgcagggtactgatggaggacactatgacggaagtaaaggcatacaaaaaggagctggaggaacagctgggcccagtggcggaggagacacgggccaggctggctaaagaggtgcaggcggcacaggcccgtctgggagctgacatggaggatctacgcaaccgactcgggcagtaccgcaacgaggtaaacaccatgctgggccagagcacagaggagctgcggtcgcgcctctccacacacctgcgcaagatgcgcaagcgcctgatgcgggatgcggatgatctgcagaagcgcctggcggtgtacaaggccggggcacaggagggcgccgagcgcggtgtgagtgctatccgtgagcgcctggggccactggtggagcagggtcgtcagcgcacagccaacctaggcgctggcgccgcccagcccctgcgcgatcgcgcccaggctttgagtgaccgcatccgagggcggctggaggaagtgggcaaccaggcccgagaccgcctagaggaggtgcgtgagcagatggaggaggtgcgctccaagatggaggagcagacccagcagatacgcctgcaggccgagatcttccaggcccgcatcaagggctggttcgagccgctagtggaagacatgcagcgccagtgggcaaacctaatggagaagatacaggcctctgtggctaccaactccattgcctccaccacagtgcccctggagaatcaatgatcatccctcacctacgccctgccgcaacatccatgaccagccaggtggccctgtcccaagcaccactctggccctctggtggcccttgcttaataaagattctccaagcacgttctgagtctctgtgagtgattccacacagcttcagcctcagtttatcgtttctgcctgacatagcacacattccatggccatgtctgtagaaggaggtcatgtgctttagttcaagtctagtttggtctgctctgctttggttttgcagtaatagagataaaacctaggagctagcaacgccaaaggagaagtgctctacctctgagccccgcccgcaacccttcaccgg

SEQ ID NO: 97 - DNA, 726 bp, Artificial Sequence
  Description of Artificial Sequence a sequence including the coding region of third exon in rat Apo E gene and a termination codon at 3′ end
  CDS (1)..(726)
gtactgatggaggacactatgacggaagtaaaggcatacaaaaaggagctggaggaacagctgggcccagtggcggaggagacacgggccaggctggctaaagaggtgcaggcggcacaggcccgtctgggagctgacatggaggatctacgcaaccgactcgggcagtaccgcaacgaggtaaacaccatgctgggccagagcacagaggagctgcggtcgcgcctctccacacacctgcgcaagatgcgcaagcgcctgatgcgggatgcggatgatctgcagaagcgcctggcggtgtacaaggccggggcacaggagggcgccgagcgcggtgtgagtgctatccgtgagcgcctggggccactggtggagcagggtcgtcagcgcacagccaacctaggcgctggcgccgcccagcccctgcgcgatcgcgcccaggctttgagtgaccgcatccgagggcggctggaggaagtgggcaaccaggcccgagaccgcctagaggaggtgcgtgagcagatggaggaggtgcgctccaagatggaggagcagacccagcagatacgcctgcaggccgagatcttccaggcccgcatcaagggctggttcgagccgctagtggaagacatgcagcgccagtgggcaaacctaatggagaagatacaggcctctgtggctaccaactccattgcctccaccacagtgcccctggagaatcaatga

SEQ ID NO: 98 - DNA, 47 bp, Artificial Sequence
  Description of Artificial Sequence primer
ccctaagcttatcgcatgccaggactgtccttacgaacctgatccac

SEQ ID NO: 99 - DNA, 37 bp, Artificial Sequence
  Description of Artificial Sequence primer
gggtaagcttgagcttcagcctctcttttctcgagag

SEQ ID NO: 100 - DNA, 60 bp, Artificial Sequence
  Description of Artificial Sequence primer
gaagaaggggtatctctcgagaaaagagaggctgaagctgtactgatggaggacactatg

SEQ ID NO: 101 - DNA, 60 bp, Artificial Sequence
  Description of Artificial Sequence primer
gaactgtgtttggtggatcaggttcgtaaggacagtcctgttgattctccaggggcactg

SEQ ID NO: 102 - DNA, 63 bp, Artificial Sequence
  Description of Artificial Sequence primer
gaactgtgtttggtggatcaggttcgtaaggacagtcctgtcattgattctccaggggcactg

(19) United States
(12) Patent Application Publication
(10) Pub. No.: US 2007/0248967 A1
(43) Pub. Date: Oct. 25, 2007

(54) Reporter Assay Using Secrectory Luminescent Enzymes

(75) Inventors: Satoru Ohgiya, Hokkaido (JP); Yuki Tochigi, Hokkaido (JP); Takehiko Sahara, Hokkaido (JP); Yoshihiro Ohmiya, Osaka (JP)

Correspondence Address: SUGHRUE MION, PLLC, 2100 PENNSYLVANIA AVENUE, N.W., SUITE 800, WASHINGTON, DC, 20037, US

(73) Assignee: NATIONAL INSTITUTE OF ADVANCED INDUSTRIAL SCIENCE, Tokyo (JP)

(21) Appl. No.: 11/660,058

(22) PCT Filed: Jun. 9, 2006
(86) PCT No.: PCT/JP06/11597

§ 371 Date: Jun. 29, 2007

(30) Foreign Application Priority Data
Jun. 9, 2005 (JP) 2005-169768

(51) Int. Cl. C12Q 1/68

(52) U.S. Cl. 435/6

(57) ABSTRACT

It is an objective of the present invention to provide a method for convenient and highly sensitive reporter assay. A secretory luminescent enzyme is used as a reporter protein so as to evaluate the expression, functions, transcriptional activity, or transcriptional control functions of a foreign gene or a foreign DNA fragment.

### TECHNICAL FIELD

The present invention relates to a reporter assay method using, for example, a secretory luminescent enzyme as a reporter protein.

### BACKGROUND ART

Reporter assay is, for example, a method for directly or indirectly measuring an amount of synthesized mRNA. Such mRNA has previously been transcribed from a gene encoding a reporter protein (hereafter to be referred to as a “reporter gene”) by the function of a DNA sequence such as a promoter that is necessary for transcriptional initiation. In general, upon reporter assay, a specific reporter gene is linked to the 3′ end downstream of a promoter and the resultant is used as a plasmid or is inserted into a chromosome, so that a transformant is constructed.

In general, upon reporter assay, it is not easy to quantify mRNA synthesized from a specific reporter gene that has been linked to the 3′ end downstream of a promoter. Thus, in many cases, the amount of a protein that has been synthesized according to information in mRNA is measured. At such time, in order to conveniently measure the amount of a protein synthesized, it is common to use a method that employs an enzyme as a reporter protein, so that the enzyme activity level may be regarded as a value indicating the relative value of the amount of mRNA synthesized. It has been generally accepted that the final enzyme activity measured correlates with the promoter transcriptional activity in the case of reporter assay.

Hitherto, there have been many studies that employ the reporter assay. Examples of such studies include elucidation of the control mechanism of a specific promoter and identification of upstream factors of signal transduction involved in such promoter control mechanism.

In addition, reporter assay has been used to examine the presences and the amounts of specific chemical substances. For instance, the use of a dioxin receptor protein (Aryl hydrocarbon receptor (AhR)) in reporter assay has been known. The receptor protein has a function of promoting the transcriptional activity of genes such as CYP1A1 (a cytochrome P450 isozyme) by binding to dioxin. Such complex of dioxin and a dioxin receptor protein moves to the nucleus and binds to a target sequence (e.g., a sequence known as “XRE”) so as to activate transcription of a gene linked to the target sequence. In this case, a cell expressing a dioxin receptor protein is prepared. In addition, a reporter plasmid containing a promoter having a target sequence of a dioxin receptor protein is prepared. A specific reporter gene is located at the 3′ end downstream of the promoter in the reporter plasmid. Then, the reporter plasmid is introduced into the cell expressing a dioxin receptor protein. When dioxin is added to a culture solution of the cell, dioxin permeates the cell so as to bind to a dioxin receptor. The complex of dioxin and a dioxin receptor protein moves to the nucleus and binds to a target sequence in the reporter plasmid. Thus, transcription of the reporter gene is activated. As described above, it is possible to determine the presence or the amount of dioxin based on the production of reporter protein or the amount of reporter protein produced via the steps of transcription from a reporter gene to mRNA and translation into a reporter protein (Kawanishi, M., Sakamoto, M., Ito, A., Kishi, K., Yagi, T. (2003) Mut. Res. 540, 99-105). Furthermore, a system for detecting environmental hormones based on a combination of an endocrine disruptor (hereafter to be referred to as an “environmental hormone”) and an estrogen receptor that binds thereto has been developed.

In addition, as a representative method for examining interaction between proteins, an experimental system known as a two-hybrid system has been widely used. Usually, such experimental system also employs reporter assay (Jung, J., Ishida, K., and Nishihara, T. (2004) Life Sci. 74, 3065-3074).

Considering the systems that use reporter assay described above, in any system in which intracellular transcriptional activation is induced, measurement can be carried out with the use of reporter assay.

In addition, a method for detecting gene mutation using reporter assay has been proposed. Firstly, a gene of interest prepared from humans or the like by PCR is fused with a reporter gene. Then, the fusion gene is expressed. Thus, an abnormal termination codon or the like that exists in the gene of interest can be detected based on detection of the presence of a reporter protein or related enzyme activity (Zhang, C. L., Tada, M., Kobayashi, H., Nozaki, M., Moriuchi, T. and Abe, H. (2000) Oncogene 19, 4346-4353). Moreover, a method for screening for a protein having a signal peptide that is necessary for its secretion with the use of reporter assay has been disclosed (Patent Document 1).

Components used in reporter assay can be roughly divided into two categories. The first type of component causes transcriptional activation. Basically, such a component comprises a promoter or a promoter containing a DNA sequence involved in transcriptional activation/repression, and a receptor and/or a coactivator that promotes transcriptional activation of a promoter. In some cases, for instance, when reporter assay is used for detection of gene mutation as described above, a gene having an abnormal termination codon or the like is the first component.

The second type of component allows a measurement of transcriptional activation. Basically, the component comprises a reporter protein. The first component can differ depending on the subject to be measured. However, the second component is essentially versatile. That is, a specific reporter protein can be commonly used for various types of reporter assay. As described above, if an improved reporter protein is developed and used instead of a reporter protein that has been conventionally used for reporter assay, an advanced type of reporter assay can be developed.

Hitherto, reporter assay has been constructed using various types of hosts such as Escherichia coli, yeasts, and cultured cells. The same basic principles of reporter assay can be applied to these hosts. However, the most important point in terms of the selection of hosts is whether or not activation of a promoter or the like to be analyzed (transcriptional activation) can be reestablished, and whether or not a reporter protein is expressed. For instance, when dioxin response in humans is examined using reporter assay, a transformant expressing a human dioxin receptor is constructed. In such case, when a prokaryote such as Escherichia coli is used as a host, it is generally difficult to achieve the expression of a human protein. Moreover, since the intracellular environment of E. coli differs significantly from that of human cells which are eukaryotic cells, it is impossible to construct an appropriate transformant that can be used for reporter assay for E. coli. Meanwhile, cultured cells are similar to human cells in terms of cellular environment. Thus, cultured cells are often used as hosts for reporter assay. However, in general, expensive fetal bovine serum is used for the culture of such cells, resulting in the increased cost. Further, in such case, the cell growth rate is very slow compared with cases in which microorganisms are used, so that the experiment becomes lengthy, which is problematic.

Meanwhile, compared with Escherichia coli and cultured cells, in the case of yeasts, the growth rate is rapid and a less expensive medium can be used for culture. Further, yeasts are eukaryotic cells, like those in humans. Thus, the intracellular environment is very similar to that of humans. Therefore, it has been known that production of human proteins can be easily carried out using yeasts. In view of such advantageous points, various types of reporter assay using yeasts have been proposed. Representative examples thereof are reporter assay for detection of dioxin or environmental hormones and a two-hybrid method for protein interaction analysis.

In the case of yeast reporter assay, Escherichia coli-derived β-galactosidase has been conventionally used as a reporter protein. In addition, recently, firefly luciferase and renilla luciferase have been used as reporter proteins (Non-Patent Document 1). In the case of all such reporter proteins, the amount of a reporter protein is measured based on enzyme activity. Further, a jellyfish-derived green fluorescent protein (GFP) and a mutant thereof have been used as reporter proteins. In the case of GFP, the amount of the reporter protein is measured based on fluorescence intensity (Non-Patent Document 2).

All of the above reporter proteins are intracellularly expressed. In order to evaluate the amounts of the above reporter proteins (other than GFP) produced as a result of enzyme activity, it is essential to carry out cell harvest via centrifugation and cell disruption using ultrasonic waves, detergents, organic solvents, and the like (or alternatively, to carry out an operation for enhancing cellular permeability). Such operations are not adequate for the processing of numerous samples. Specifically, as long as these reporter proteins are used, it is impossible to construct so-called high-throughput assay whereby numerous samples are processed.

On the other hand, a technique is known wherein firefly luciferase is allowed to be expressed in a cell or peroxisome, resulting in uptake of luciferin serving as a substrate through a medium (Non-Patent Document 3). However, in accordance with such technique, uptake of a substrate is a rate-limiting factor, so that it cannot be expected to obtain sufficient activity. In addition, in the case of reporter assay wherein GFP is used as a reporter protein, measurement can be carried out while GFP is intracellularly expressed. Thus, such reporter assay is advantageous because neither cell recovery nor disruption is required. However, when GFP is used as a reporter protein, a high background intensity is obtained upon measurement of fluorescence intensity due to properties of GFP, which is problematic. Such high background intensity is derived from scattered light or the like generated from a fluorescent substance or a yeast cell in a medium. Meanwhile, in order to avoid the obtaining of such background intensity, a method using a flow cytometer known as FACS has been known. However, in such case, the required apparatus itself is very expensive.

As described above, there have been no reports of convenient and highly sensitive reporter proteins that can be used for yeast reporter assay. An ideal convenient reporter protein is a secretory protein that can be used without cell harvest or cell disruption. Also, the most appropriate protein as an ideal high-sensitivity reporter protein is a protein that causes luminescence from which a low background intensity is obtained based on measurement principles.

Patent Document 2 and Non-Patent Document 4 disclose that a gene encoding a Cypridina noctiluca-derived luciferase is subjected to cloning, resulting in extracellular secretion of the luciferase from mammalian cells with good efficiency. However, there have been no reports of yeast reporter assay employing secretory luminescent enzymes, including a Cypridina noctiluca-derived luciferase or other secretory luciferases, as reporter proteins.

[Patent Document 1] JP Patent Publication (Kohyo) No. 2003-530106 A

[Patent Document 2] JP Patent Publication (Kokai) No. 2004-187652 A

[Non-Patent Document 1] Harger, J. W. and Dinman J. D., “RNA,” 2003, vol. 9, pp. 1019-1024

[Non-Patent Document 2] Bovee, T. F. H., Helsdingen, R. J. R., Koks, P. D., Kuiper, H. A., Hoogenboom, R. L. A. P., and Keijer, J., “Gene,” 2004, vol. 325, pp. 187-200

[Non-Patent Document 3] Leskinen P., Virtaq, M., and Karp, M., “Yeast,” 2003, vol. 20, pp. 1109-1113

[Non-Patent Document 4] Nakajima Y., Kobayashi, K., Yamagishi, K., Enomoto, T., and Ohmiya, Y., “Bioscience, Biotechnology, and Biochemistry,” 2004, vol. 68, pp. 565-570

### DISCLOSURE OF THE INVENTION

In view of the above circumstances, it is an objective of the present invention to provide a method for convenient and highly sensitive reporter assay.

In particular, the inventors of the present invention have examined the direct use of a culture or culture supernatant for reporter assay. In order to carry out reporter assay with the direct use of a culture, it is necessary to use a secretory luminescent enzyme as a reporter protein.

As a result of intensive studies in order to solve. the above problems, the inventors of the present invention have found that a gene encoding a secretory luminescent enzyme is introduced into a host, the culture or culture supernatant of the obtained transformant is allowed to come into contact with the substrate of the secretory luminescent enzyme, and the enzyme activity of the secretory luminescent enzyme is measured, such that the expression, functions, transcriptional activity, or transcriptional control functions of the foreign gene or foreign DNA fragment that has been introduced into the host can be efficiently evaluated. This has led to the completion of the present invention.

The present invention encompasses the following (1) to (29):

(1) a reporter assay method, comprising: a first step of introducing a gene encoding a secretory luminescent enzyme into a host; a second step of allowing the culture or culture supernatant of the transformant obtained in the first step to come into contact with the substrate of the secretory luminescent enzyme; and a third step of measuring the enzyme activity of the secretory luminescent enzyme, wherein the expression, functions, transcriptional activity, or transcriptional control functions of the foreign gene or foreign DNA fragment that has been introduced into the host is evaluated based on the enzyme activity of the secretory luminescent enzyme;

(2) the reporter assay method described in (1), wherein the foreign gene is linked to the gene encoding a secretory luminescent enzyme or is inserted between a gene encoding a secretory signal peptide and a gene encoding a mature protein in the gene encoding a secretory luminescent enzyme;

(3) the reporter assay method described in (1), wherein the foreign gene is expressed in the host;

(4) the reporter assay method described in (1), wherein the foreign DNA fragment is linked to the gene encoding a secretory luminescent enzyme;

(5) the reporter assay method described in (1), wherein the secretory luminescent enzyme is a secretory luciferase;

(6) the reporter assay method described in (5), wherein the secretory luciferase is Cypridina luciferase;

(7) the reporter assay method described in (6), wherein the Cypridina luciferase is a Cypridina noctiluca-derived luciferase;

(8) the reporter assay method described in (1), wherein the secretory luminescent enzyme is a fusion protein of a secretory signal peptide that functions in the host and a mature protein of a secretory luminescent enzyme;

(9) the reporter assay method described in (1), wherein the host is a yeast;

(10) the reporter assay method described in (9), wherein the yeast is Saccharomyces cerevisiae;

(11) the reporter assay method described in (9), wherein a transformant of the yeast is cultured under conditions of pH 3.5 to 6.5;

(12) a reporter assay method, comprising: a first step of introducing into a host DNA in which a gene encoding a secretory signal peptide or secretory protein is linked to the upstream of a gene encoding a mature protein of a secretory luminescent enzyme; a second step of allowing the culture or culture supernatant of the transformant obtained in the first step to come into contact with the substrate of the secretory luminescent enzyme; and a third step of measuring the enzyme activity of the secretory luminescent enzyme; wherein the secretion capacity of the secretory signal peptide or secretory protein is evaluated based on the enzyme activity of the secretory luminescent enzyme;

(13) the reporter assay method described in (12), wherein the secretory luminescent enzyme is a secretory luciferase;

(14) the reporter assay method described in (13), wherein the secretory luciferase is Cypridina luciferase;

(15) the reporter assay method described in (14), wherein the Cypridina luciferase is a Cypridina noctiluca-derived luciferase;

(16) the reporter assay method described in (12), wherein the host is a yeast;

(17) the reporter assay method described in (16), wherein the yeast is Saccharomyces cerevisiae;

(18) the reporter assay method described in (16), wherein a transformant of the yeast is cultured under conditions of pH 3.5 to 6.5;

(19) a reporter assay method, comprising: a first step of introducing a gene encoding a secretory luminescent enzyme into a host; a second step of allowing the culture or culture supernatant of the transformant obtained in the first step to come into contact with the substrate of the secretory luminescent enzyme; and a third step of measuring the enzyme activity of the secretory luminescent enzyme; wherein chemical or physical change is measured based on the enzyme activity of the secretory luminescent enzyme;

(20) the reporter assay method described in (19), wherein the method comprises a step of exposing the transformant to a subject of chemical or physical change after the first step;

(21) the reporter assay method described in (19), wherein a foreign DNA fragment that is responsive to the chemical or physical change is linked to the gene encoding a secretory luminescent enzyme;

(22) the reporter assay method described in (19), wherein a foreign DNA fragment that interacts with a protein that is responsive to the chemical or physical change is linked to the gene encoding a secretory luminescent enzyme;

(23) the reporter assay method described in (22), wherein the protein that is responsive to the chemical or physical change is encoded by a foreign gene;

(24) the reporter assay method described in (19), wherein the secretory luminescent enzyme is a secretory luciferase;

(25) the reporter assay method described in (24), wherein the secretory luciferase is Cypridina luciferase;

(26) the reporter assay method described in (25), wherein the Cypridina luciferase is a Cypridina noctiluca-derived luciferase;

(27) the reporter assay method described in (19), wherein the host is a yeast;

(28) the reporter assay method described in (27), wherein the yeast is Saccharomyces cerevisiae; and

(29) the reporter assay method described in (27), wherein a transformant of the yeast is cultured under conditions of pH 3.5 to 6.5.

In accordance with the present invention, a convenient and highly sensitive reporter assay method is provided. Specifically, in accordance with the present invention, reporter assay can be carried out efficiently with the use of yeasts and secretory luminescent enzymes such as secretory luciferase.

This description includes part or all of the contents as disclosed in the description of Japanese Patent Application No. 2005-169768, which is a priority document of the present application.

### BRIEF DESCRIPTION OF THE DRAWINGS

FIG. 1 shows results of the examination of culture conditions in secretory expression of CLuc using a CLuc-expressing transformant.

FIG. 2A shows results of CLuc activity measurement using Cypridina-luciferin-diluted solutions that were diluted with buffer solutions with different pH levels.

FIG. 2B shows results of CLuc activity measurement using Cypridina-luciferin-diluted solutions that were diluted with Tris-HCl (pH 7.4) solutions at different final concentrations.

FIG. 3 shows results of CLuc activity measurement using culture supernatants of a CLuc-expressing transformant that were sequentially diluted relative to different final concentrations of luciferin in reaction solutions.

FIG. 4 shows the relative residual activity of CLuc measured after incubation of culture supernatants of a CLuc-expressing transformant at different temperatures for a certain period of time.

FIG. 5 shows results of examination of the linearity and the dynamic range of a culture supernatant of a CLuc-expressing transformant upon CLuc activity measurement.

FIG. 6 shows results of examination of inhibitory effects of different chemical substances with respect to CLuc upon CLuc activity measurement.

FIG. 7A shows results of CLuc activity measurement (shown as RLU) using culture solutions or culture supernatants of CLuc-expressing transformants at different turbidity levels.

FIG. 7B shows results of CLuc activity measurement (shown as RLU/OD) using a culture solution and a culture supernatant of a CLuc-expressing transformant.

FIG. 8 shows results of CLuc activity measurement using culture solution samples obtained by sampling from a culture solution of a CLuc-expressing transformant and incubating the samples for differing periods of time at 25° C.

FIG. 9 shows results of CLuc activity measurement using a culture solution of a transformant having αCLuc-encoding DNA linked to a TDH3 promoter cultured in a 96-deep well plate.

FIG. 10 shows results of mCLuc activity measurement using culture solutions of transformants each having mCLuc-encoding DNA linked to the corresponding promoter shown at the bottom of the figure.

FIG. 11 shows results of β-galactosidase activity measurement using culture solutions of transformants each having β-galactosidase-encoding DNA linked to the corresponding promoter shown at the bottom of the figure.

FIG. 12 shows mCLuc mRNA contents that were measured by real-time PCR so as to be standardized based on TDH3 mRNA contents.

FIG. 13A shows results of mCLuc activity measurement using culture solutions of transformants each having mCLuc-encoding DNA linked to the corresponding promoter shown at the bottom of the figure in the presence and absence of copper ions.

FIG. 13B shows results of β-galactosidase activity measurement using culture solutions of transformants each having β-galactosidase-encoding DNA linked to the corresponding promoter shown at the bottom of the figure in the presence and absence of copper ions.

FIG. 14A shows results of mCLuc activity measurement using culture solutions of transformants each having mCLuc-encoding DNA linked to the corresponding promoter shown at the bottom of the figure in the presence and absence of galactose.

FIG. 14B shows results of β-galactosidase activity measurement using culture solutions of transformants each having β-galactosidase-encoding DNA linked to the corresponding promoter shown at the bottom of the figure in the presence and absence of galactose.

FIG. 15 shows results of mCLuc activity measurement using culture solutions of transformants each having mCLuc-encoding DNA linked to a different TDH3 promoters, such promoters having various lengths, shown in the left panel (each promoter sequence having a known cis sequence as shown in the figure).

FIG. 16 shows results of mCLuc activity measurement using culture solutions of transformants each having mCLuc-encoding DNA linked to a different GAL1 promoters, such promoters having various lengths, shown in the left panel (each promoter sequence having a known cis sequence as shown in the figure).

FIG. 17 shows results of CLuc activity measurement using culture solutions of transformants each having a reporter plasmid in which the corresponding DNA shown at the bottom of the figure (equivalent to a code region contained in the third exon of rat ApoE gene) has been inserted between α-factor secretory signal peptide DNA and a mature CLuc DNA in αCLuc-encoding DNA. In addition, ApoE (+), ApoE (−), and ApoE (±) denote DNA not containing a termination codon, DNA containing a termination codon, and DNA obtained by samples containing an equal amount of each DNA, respectively.

### BEST MODE FOR CARRYING OUT THE INVENTION

Hereafter, the present invention will be described in greater detail.

The reporter assay method of the present invention is a method for evaluating the expression, functions, transcriptional activity, or transcriptional control functions of a foreign gene or foreign DNA fragment using a secretory luminescent enzyme as a reporter protein. In accordance with the method of the present invention, a gene encoding a secretory luminescent enzyme (hereafter to be referred to as “secretory luminescent enzyme gene”) is first introduced into a host. Then, the culture or culture supernatant of the obtained transformant is allowed to come into contact with a substrate of a secretory luminescent enzyme under conditions that allow an enzyme reaction of the secretory luminescent enzyme to take place. Thereafter, the enzyme activity of the secretory luminescent enzyme is measured. The method of the present invention is intended to evaluate the expression, functions, transcriptional activity, or transcriptional control functions of the foreign gene or foreign DNA fragment that has been introduced into the host based on enzyme activity.

Herein, the term “secretory luminescent enzyme” indicates an enzyme that is secreted to the outside of a cell membrane or cell wall and catalyzes a luminous reaction upon degradation of a substrate. Examples of a secretory luminescent enzyme/substrate that can be used in the reporter assay method of the present invention include secretory luciferase/luciferin and a secretory phosphatase/1,2-dioxetane derivative.

Secretory luciferases catalyze oxidation of luciferin, which is a substrate, with oxygen molecules. At such time, reaction energy that has been produced causes the generation of an oxidation product (oxyluciferin) at an excited state. Luminescence then takes place when such product returns to the ground state. Examples of such secretory luciferases include Cypridina luciferases such as a Cypridina noctiluca-derived luciferase (Cypridina noctiluca is closely related to Cypridina) (cDNA: SEQ ID NO: 1; amino acid sequence: SEQ ID NO: 2) and a Vargula hilgendorfii-derived luciferase (cDNA: SEQ ID NO: 3; amino acid sequence: SEQ ID NO: 4), Oplophorus gracilirostris (Oplophoridae)-derived luciferase (cDNA: SEQ ID NO: 5; amino acid sequence: SEQ ID NO: 6), and a Metridia longa (of Copepoda)-derived luciferase (cDNA: SEQ ID NO: 7; amino acid sequence: SEQ ID NO: 8). In view of high efficiency of secretion, a Cypridina noctiluca-derived luciferase is particularly preferable.

In addition, a secretory phosphatase catalyzes dephosphorylation of a 1,2-dioxetane derivative (CDP-Star, CSPD; Applied Biosystems) that is a luminescent substrate. At such time, the dephosphorylated substrate is spontaneously degraded to adamantanon and luminophore. Luminescence takes place when such a luminophore at an excited state that has been produced due to reaction energy returns to the ground state. An example of such secretory phosphatase is human placental alkaline phosphatase (SEAP (cDNA: SEQ ID NO: 9; amino acid sequence: SEQ ID NO: 10)).

In general, a secretory protein containing a secretory luminescent enzyme is synthesized in the form of a precursor having a secretory signal peptide at its N-terminal. Such precursor is cleaved with a signal peptidase during a transmembrane process so that it becomes a mature protein. In accordance with the present invention, the term “mature protein” indicates a protein that is secreted to the outside of a cell membrane or cell wall. In general, a secretory signal peptide is often removed from a mature protein. Further, the mature protein of the present invention may be a mature protein from which a secretory signal peptide is not removed but from which a sequence that is presumed to correspond to a secretory signal peptide is removed.

In accordance with the reporter assay method of the present invention, a secretory luminescent enzyme may be a fusion protein of a secretory signal peptide and a nonsecretory luminescent enzyme, as long as such protein is actually secreted to the outside of a cell membrane or cell wall. In addition, a fusion protein may be used as a secretory luminescent enzyme, such fusion protein being obtained by linking the N-terminal of a mature protein of a secretory luminescent enzyme to a secretory signal peptide known to function as a secretory signal peptide in a selected host in place of an original secretory signal peptide. For instance, a secretory signal peptide that is the α-factor of Saccharomyces cerevisiae (cDNA: SEQ ID NO: 11; amino acid sequence: SEQ ID NO: 12) has been known to contribute to high secretion efficiency in yeast. In addition, an example of a secretory signal peptide in yeast is a secretory signal peptide of invertase. Meanwhile, a secretory signal peptide of a Cypridina noctiluca-derived secretory luciferase is an amino acid sequence (1^{st} to 18^{th} amino acids) of the amino acid sequence of Cypridina noctiluca-derived secretory luciferase set forth in SEQ ID NO: 2. Thus, in a case in which a yeast is selected as a host, for example, a fusion protein (cDNA: SEQ ID NO: 13; amino acid sequence: SEQ ID NO: 14) can be used as a secretory luminescent enzyme, such fusion protein being obtained by linking a secretory signal peptide of an α-factor to the N-terminal of a mature protein of Cypridina noctiluca-derived secretory luciferase.

Nucleotide sequences of the secretory luminescent enzyme genes described above and amino acid sequences corresponding thereto are not limited to the above nucleotide sequences set forth in the above SEQ ID NOS or the amino acid sequences corresponding thereto. Each secretory luminescent enzyme may have an amino acid sequence derived from the amino acid sequence represented by any of the amino acid sequences set forth in the above SEQ ID NOS by substitution, deletion, or addition of one or more amino acids (e.g., 1 to 10 or 1 to 5 amino acids). In addition, such secretory luminescent enzyme may be secreted and have enzyme activity of an original secretory luminescent enzyme. Further, based on the frequency of use of a codon of a host to be transformed and the like, a secretory luminescent enzyme gene obtained by optimizing the nucleotide sequence of the secretory luminescent enzyme gene set forth in one of the above SEQ ID NOS can be used. An example of such secretory luminescent enzyme gene is a synthetic gene (cDNA: SEQ ID NO: 15) that is obtained by optimizing the Cypridina noctiluca-derived luciferase gene (cDNA: SEQ ID NO: 1) to be used in Saccharomyces cerevisiae. In addition, the amino acid sequence of a luciferase encoded by the gene is identical to the amino acid sequence of a wild-type luciferase (SEQ ID NO: 2).

A foreign gene used in the reporter assay method of the present invention may be a gene encoding any type of protein or peptide. The present invention is described in the first to third embodiments described below, for example, according to the types of foreign genes.

In the first embodiment, when a protein is evaluated in terms of stability, functions, and the like when the protein has an amino acid sequence with an abnormal termination codon or with mutation such as a deletion, substitution, or addition, a gene encoding the protein to be evaluated is designated as a foreign gene. In addition, when a gene encoding a protein is evaluated in terms of abnormalities, a gene encoding the protein of interest is designated as a foreign gene, such protein being derived from DNA or RNA prepared from a sample such as human blood. In the above cases, a foreign gene is linked to the 5′ end upstream of a secretory luminescent enzyme gene or is inserted between a gene encoding a secretory signal peptide and a gene encoding a mature protein in a secretory luminescent enzyme gene. In particular, it is preferable to link a foreign gene between a gene encoding a secretory signal peptide and a gene encoding a mature protein in a secretory luminescent enzyme gene such that secretory luminescent enzyme functions are unlikely to be lost. In accordance with the reporter assay method of the present invention, because of the positional relationship between a secretory luminescent enzyme gene and a foreign gene described above, the enzyme activity level of a secretory luminescent enzyme is allowed to correlate with the expression, activity, and functions of a foreign gene. For instance, a gene encoding a specific normal protein is designated as a control foreign gene. On the other hand, a gene with which the presence or absence of an abnormal termination codon in a gene is evaluated (hereafter to be referred to as “test gene”) is designated as a foreign gene. When the enzyme activity level is low or is not detected when using a test gene compared with the enzyme activity level detected when using a control foreign gene, it is possible to evaluate the presence of abnormalities in a test gene without determining the nucleotide sequence.

In the second embodiment, an example of the foreign gene is a gene encoding a foreign transcription factor or transcriptional suppressor. In such case, when a protein to be examined in terms of transcriptional control functions is derived from a host, a “foreign gene” may be a gene derived from the host. Alternatively, the gene may be linked to the entirety or a part of another gene encoding a transcription factor or transcriptional suppressor so that a gene encoding the fusion protein is designated as a foreign gene. For instance, when the reporter assay method of the present invention is used for a two-hybrid system using yeast, a gene encoding a bait protein and a gene encoding a protein (test protein) that is examined concerning whether or not it interacts with the bait protein are designated as foreign genes. In such case, such foreign genes are not directly linked to a secretory luminescent enzyme gene but exist at another site of the same plasmid or another plasmid or chromosome so as to be expressed in a host. In accordance with the reporter assay method of the present invention, with the use of the enzyme activity level of a secretory luminescent enzyme, transcriptional control functions of a protein encoded by a foreign gene or the strength of interaction between a bait protein and test protein in a two-hybrid system can be evaluated. Further, in accordance with the reporter assay method of the present invention, with the use of the enzyme activity level of a secretory luminescent enzyme, the binding capacity of DNA involved in transcriptional activation and the strength of interaction with a cofactor or RNA polymerase, for example, in a one-hybrid system can be evaluated.

In the third embodiment, a gene encoding a protein subjected to screening is designated as a foreign gene when the reporter assay method of the present invention is used for screening for a protein that is fixed to a cell membrane or cell wall (hereafter to be referred to as “cell membrane protein or cell wall protein”) from many proteins. In such case, the foreign gene is linked to the 5′ upstream region or the 3′ downstream region of a secretory luminescent enzyme gene. Also, in such case, the foreign gene is linked to the secretory luminescent enzyme gene in a manner such that a secretory luminescent enzyme is fixed to the outer surface of the cell membrane or cell wall. When the foreign gene is a gene encoding a cell membrane protein or cell wall protein, the cell membrane protein or cell wall protein is fixed to a cell membrane or cell wall of a host together with the secretory luminescent enzyme. Based on the enzyme activity level of the fixed secretory luminescent enzyme, the cell membrane protein or cell wall protein encoded by the foreign gene can be screened for.

In addition, either the N-terminal or the C-terminal of a cell membrane protein or a cell wall protein may exist outside of a cell membrane or cell wall. Thus, in accordance with the aforementioned screening with the use of the reporter assay method of the present invention, it is possible to evaluate whether or not either the N-terminal or the C-terminal of a cell membrane protein or a cell wall protein exists outside of a cell membrane or cell wall based on the enzyme activity level of the secretory luminescent enzyme. Further, in accordance with the aforementioned screening with the use of the reporter assay method of the present invention, the expression level of a cell membrane protein or a cell wall protein can be evaluated based on the enzyme activity level of the secretory luminescent enzyme.

Meanwhile, an example of a foreign DNA fragment is a promoter, or a DNA fragment or synthetic DNA that has a sequence causing transcriptional activation or repression, which is generally called a cis-sequence. Further, a foreign DNA fragment may be a DNA fragment having a sequence that contributes to mRNA stability or a sequence that affects translational efficiency. In accordance with the reporter assay method of the present invention, a promoter used as a foreign DNA fragment may be a promoter derived from any organism or a combination of artificial cis sequences, or.it may have any sequence, such as an artificial sequence, as long as the foreign DNA fragment functions as a promoter. For instance, when a foreign DNA fragment is a promoter, the promoter is linked to the 5′ end upstream of a secretory luminescent enzyme gene. In such case, in accordance with the reporter assay method of the present invention, the obtained enzyme activity level of the secretory luminescent enzyme can be evaluated as a relative value of the transcriptional activation level of the promoter used as a foreign DNA fragment. In addition, when a foreign DNA fragment has a cis sequence that causes transcriptional activation or transcriptional repression, a sequence that contributes to mRNA stability, or a sequence that affects translational efficiency, the foreign DNA fragment may be located at any site with respect to a secretory luminescent enzyme gene as long as the fragment can function. In such case, in accordance with the reporter assay method of the present invention, based on the obtained enzyme activity level of the secretory luminescent enzyme, the aforementioned sequence in a foreign DNA fragment can be evaluated in terms of transcriptional control function (e.g., transcriptional activation or transcriptional repression), contribution to mRNA stability, or influence on translational efficiency. In addition, another foreign DNA fragment may be linked to a site where it can be controlled with respect to a secretory luminescent enzyme gene.

The host is not particularly limited, as long as a secretory luminescent enzyme gene and a foreign gene or a foreign DNA fragment can function in the host. Examples thereof include: yeast; bacteria belonging to the genus Escherichia, including Escherichia coli, the genus Bacillus, including Bacillus subtilis, or the genus Pseudomonas, including Pseudomonas putida, and the like; animal cells such as COS cells; insect cells such as Sf9; and plants belonging to the genus Brassicaceae or the like. In addition, any type of yeast may be used. Examples of such yeast include Saccharomyces cerevisiae, Shizosaccharomyces pombe, Pichia pastoris, Candida albicans, and Hansenula polymorpha. Among them, Saccharomyces cerevisiae is particularly preferable.

In accordance with the reporter assay method of the present invention, a secretory luminescent enzyme gene and a foreign gene or foreign DNA fragment are first prepared. A secretory luminescent enzyme gene, a foreign gene, and a foreign DNA fragment (hereafter to be referred to as “gene or the like”) can readily be obtained by PCR using genomic DNA or the like of an organism containing the gene or the like (as a template) and primers complementary to nucleotide sequences at both ends of the region.

Once the nucleotide sequence of a gene or the like is determined, such gene or the like can be obtained by chemical synthesis, by PCR using a probe subjected to cloning as a template, or by hybridization using a DNA fragment having the nucleotide sequence as a probe. Moreover, a mutant of a gene or the like, which has functions equivalent to those of the gene or the like before mutation, can be synthesized by site-directed mutagenesis or the like.

In addition, mutagenesis of a gene or the like can be carried out by conventional methods such as the Kunkel method and the gapped duplex method, and by methods similar thereto. For instance, mutagenesis may be carried out using a mutagenesis kit for site-directed mutagenesis (e.g., Mutant-K or Mutant-G (TAKARA)) or an LA PCR in vitro Mutagenesis series kit (TAKARA).

Subsequently, when the foreign gene or foreign DNA fragment is linked to the secretory luminescent enzyme gene, DNA is prepared in which the foreign gene or foreign DNA fragment is linked to the secretory luminescent enzyme gene. In addition, when the foreign gene or foreign DNA fragment is inserted between a gene encoding a secretory signal peptide and a gene encoding a mature protein in the secretory luminescent enzyme gene, DNA is prepared in which the foreign gene or foreign DNA fragment is inserted between a gene encoding a secretory signal peptide and a gene encoding a mature protein in the secretory luminescent enzyme gene. Such DNA is DNA subjected to linkage or insertion as described above, or a vector containing such DNA is used.

A method for linking a foreign gene or foreign DNA fragment to a secretory luminescent enzyme gene that can be used is a method for cleaving a secretory luminescent enzyme gene and a foreign gene or foreign DNA fragment that have been separately purified with an adequate restriction enzyme, followed by linkage. In addition, another such method is a method for linking a secretory luminescent enzyme gene to a foreign gene or foreign DNA fragment via in vitro linkage using PCR or in vivo linkage using a yeast or the like, wherein the secretory luminescent enzyme gene and a foreign gene or foreign DNA fragment have a homologous region.

In addition, the foreign gene or foreign DNA fragment can be inserted between a gene encoding a secretory signal peptide and a gene encoding a mature protein in the secretory luminescent enzyme gene in accordance with the aforementioned method for linking a foreign gene or foreign DNA fragment to a secretory luminescent enzyme gene.

A vector containing DNA obtained by linking a secretory luminescent enzyme gene to a foreign gene or foreign DNA fragment or DNA obtained by inserting a foreign gene or a foreign DNA fragment between a gene encoding a secretory signal peptide and a gene encoding a mature protein in a secretory luminescent enzyme gene (hereafter to be referred to as “DNA of the present invention”) can be obtained by inserting DNA of the present invention into an adequate vector. Such vector is not particularly limited, as long as it can be replicated in a host. Examples thereof include a plasmid, a shuttle vector, and a helper plasmid. In addition, if such vector has no replication capacity, a DNA fragment that can be replicated when it is inserted into a chromosome of a host, for example, may be used.

Examples of a plasmid DNA include an Escherichia coli-derived plasmid (e.g., pBR322, pBR325, pUC118, pUC119, pUC18, pUC19, and pBluescript), a Bacillus subtilis-derived plasmid (e.g., pUB110 and pTP5), and a yeast-derived plasmid (e.g., a YEp system such as YEp13 and a YCp system such as YCp50). Examples of a phage DNA include kphage (e.g., Charon4A, Charon21A, EMBL3, EMBL4, λgt10, λgt11, and λZAP). Further, animal viruses such as retroviruses and vaccinia viruses, and insect virus vectors such as baculoviruses, for example, may be used.

A method for inserting DNA of the present invention into a vector can be carried out in accordance with the aforementioned method for linking a foreign gene or foreign DNA fragment to a secretory luminescent enzyme gene.

Further, in accordance with the reporter assay method of the present invention, a transformant is produced by introducing DNA of the present invention or a vector containing DNA of the present invention (hereafter to be referred to as “vector or the like of the present invention”) into a host. Likewise, when a secretory luminescent enzyme gene and a foreign gene exist in different vectors or the like, a transformant is produced by introducing a vector containing a secretory luminescent enzyme gene and a vector containing a foreign gene into a single host.

A method for introducing the vector or the like of the present invention into a yeast is not particularly limited, as long as it is a method for introducing DNA into a yeast. Examples of such method include an electroporation method, a spheroplast method, and a lithium acetate method. Also, a yeast transformation method may be used, wherein a vector obtained from the YIp system or the like or a DNA sequence complementary to an arbitrary site on a chromosome is substituted or inserted into a chromosome. Further, a method for introducing the vector or the like of the present invention into a yeast may be carried out in accordance with any method described in general experiment manuals, scientific papers, or the like.

A method for introducing the vector or the like of the present invention into a bacterium is not particularly limited as long as it allows DNA to be introduced into a bacterium. Examples of such method include a method using calcium ions and an electroporation method.

When an animal cell is a host, monkey COS-7 cells, Vero cells, Chinese hamster ovary cells (CHO cells), mouse L cells, and the like can be used. Examples of a method for introducing the vector or the like of the present invention into an animal cell include an electroporation method, a calcium phosphate method, and a lipofection method.

When an insect cell is a host, an Sf9 cell or the like can be used. Examples of a method for introducing the vector or the like of the present invention into an insect cell include a calcium phosphate method, a lipofection method, and an electroporation method.

When a plant is a host, the entire plant body, plant organs (e.g. leaves, petals, stems, roots, and seeds), plant tissue (e.g., epidermis, phloem, parenchyma, xylem, and vascular bundle), or a cultured cell of a plant can be used. Examples of a method for introducing a vector or the like of the present invention into a plant include an electroporation method, the Agrobacterium method, the particle gun method, and the PEG method.

It is possible to confirm whether or not the vector or the like of the present invention has been incorporated into a host by PCR, Southern hybridization, Northern hybridization, and the like. As an example, DNA is prepared from a transformant and a DNA-specific primer is designed, followed by PCR. Thereafter, an amplified product is subjected to agarose gel electrophoresis, polyacrylamide gel electrophoresis, or capillary electrophoresis. The amplified product is stained with ethidium bromide, an SYBR Green solution, or the like so that a band corresponding thereto may be detected. Accordingly, transformation is confirmed. Alternatively, PCR is carried out using a primer labeled with a fluorescent dye or the like so that an amplified product can be detected. Moreover, an amplified product is allowed to bind to a solid phase such as a microplate so that such amplified product can be confirmed based on fluorescence, an enzyme reaction, or the like.

Next, in accordance with the reporter assay method of the present invention, the obtained transformant is cultured under conditions that allow it to be grown. Further, when a culture of a transformant is subjected to the measurement of the enzyme activity in accordance with the method of the present invention, a transformant is cultured under conditions such that a secretory luminescent enzyme remains without being deactivated. For instance, upon culture of a transformed yeast into which a secretory luciferase such as Cypridina noctiluca-derived luciferase as a secretory luminescent enzyme has been introduced, the temperature is set at, for example, 4° C. to 37° C. and preferably at 20° C. to 30° C., which allow the yeast to be grown and the luciferase to remain without being deactivated. In addition, the pH of a medium may be adjusted to 3.5 to 6.5 and preferably to 5.5 to 6.0. A time period for the culture may be, for example, 1 to 120 hours and preferably 1 to 24 hours in terms of logarithmic growth phase, as long as the secretory luminescent enzyme activity can be measured.

Further, in accordance with the reporter assay method of the present invention, a culture or culture supernatant obtained after culture of a transformant is allowed to come into contact with a substrate of a secretory luminescent enzyme under conditions that allow the enzyme reaction of secretory luminescent enzyme to take place. Herein, the phrase “conditions that allow the enzyme reaction of secretory luminescent enzyme to take place” indicates conditions such that a substrate specifically binds to the active center of secretory luminescent enzyme and a complex is formed, so that the enzyme reaction proceeds. Also, herein, the phrase “come into contact” indicates a state whereby a substrate and a secretory luminescent enzyme come very close to each other in a culture or culture supernatant such that an enzyme reaction takes place. In addition, in accordance with the reporter assay method of the present invention, the term “culture” indicates a culture solution or medium containing a transformant. In accordance with the reporter assay method of the present invention, since a secretory luminescent enzyme is secreted in a medium, a culture solution or medium containing transformant can be used as it is. Alternatively, in accordance with the reporter assay method of the present invention, a culture supernatant from which a transformant has been separated by centrifugation or the like can be used.

For instance, the temperature condition that allows a culture or culture supernatant of a transformant containing a secretory luciferase such as a Cypridina noctiluca-derived luciferase to come into contact with a substrate (luciferin) is set at, for example, 0° C. to 40° C. and preferably at 15° C. to 30° C. In addition, the pH condition may be adjusted to, for example, 4.0 to 9.0 and preferably to 6.0 to 8.0. The time period during which the contact takes place (reaction time) is, for example 1 second to 30 minutes and preferably 1 to 30 seconds. In particular, substrate solutions diluted with various types of buffer solutions are added to a culture or culture supernatant such that the pH of a culture or culture supernatant can be shifted to a level at which the enzyme activity of a secretory luminescent enzyme becomes high. For instance, a luciferin solution diluted with a buffer solution such as Tris-hydrochloric acid buffer solutions (Tris-HCl: 2M or lower (preferably 50 mM to 200 mM) and pH 3.5 to 9.0 (preferably pH 7.0 to 8.0)) is added to a culture or culture supernatant containing a secretory luciferase such that the pH can be adjusted to the aforementioned pH in the contact state.

The concentration of a substrate relative to a culture or culture supernatant is adequately determined depending on a secretory luminescent enzyme and a substrate. For instance, a luciferin serving as a substrate is added to a final concentration of 0.1 μM or more and preferably of 1.25 to 2.5 μM with respect to the turbidity (e.g., absorbance at 600 nm) of a culture or culture supernatant of a transformant containing a secretory luciferase of 0.05 or more.

Next, in accordance with the reporter assay method of the present invention, the enzyme activity of a secretory luminescent enzyme is measured. A method for the measurement is adequately selected depending on a secretory luminescent enzyme. For instance, in the case of a secretory luciferase used as a secretory luminescent enzyme, a mixture of a substrate and a culture or culture supernatant of a transformant is subjected to luminescence measurement using a luminometer so that the enzyme activity is measured in relative light units (RLU). In addition, preferably, a measurement value is standardized by correcting the enzyme activity upon measurement of the activity in a manner such that the turbidity (e.g., absorbance at 600 nm) of a culture solution or culture supernatant is measured and relative light units are divided by the turbidity, such that the thus corrected value (RLU/OD) can be determined to represent the enzyme activity level. Alternatively, in order to standardize relative light units, a method wherein the ATP level of a transformant is measured and relative light units are divided by the ATP level is also preferable. Further, a method, wherein another enzyme or protein is simultaneously expressed in a transformant and the amount of the enzyme or protein is measured, such that relative light units are divided by the value of the amount for correction, may be used. Furthermore, as long as luminescence can be distinguished based on properties in terms of differences in substrates or luminescence spectra, for example, a method wherein another luminescent enzyme or a mutant of the secretory luminescent enzyme of the present invention is allowed to be expressed so that relative light units are divided by the level of luminescence derived from such luminescent enzyme for correction may also be used.

In addition, in a case in which a host is a microorganism such as Saccharomyces cerevisiae, a transformant is grown in an agar medium so that colony of the transformant is formed. For instance, in a case of a secretory luciferase is used as a secretory luminescent enzyme, a luciferin is added to an agar medium containing a transformant and the luminescence intensity of the colony is measured using, for example, a luminescence detector equipped with a CCD camera or the like, such that the enzyme activity can be measured.

In accordance with the reporter assay method of the present invention, the thus obtained enzyme activity level of secretory luminescent enzyme is allowed to correlate with the expression, functions, transcriptional activity, or transcriptional control functions of a foreign gene or foreign DNA fragment.

As described above, in accordance with the reporter assay method of the present invention, convenient and highly sensitive reporter assay can be carried out. In addition, in accordance with the reporter assay method of the present invention, a high-sensitivity luminescence measurement method whereby a low background intensity is obtained can be used with efficiency. Thus, a microassay system can be established. In accordance with the reporter assay method of the present invention, a sample to be measured can be prepared by sampling of a culture without harvest or cell disruption. Thus, such step can be automated using a robot. Specifically, for instance, 96 types of transformants are cultured with the use of a 96-deep well plate and a part of a culture is dispensed into a plate used for luminescence measurement by manual or robot operations, such that the enzyme activity can directly be measured using a luminometer that accepts a microplate. In addition, a part of the culture is taken from the 96-deep well plate so as to be dispensed into a plate used for absorbance measurement such that the turbidity used for correction can be measured using a microplate reader. In a case in which another method for correction is used, a luminescence, fluorescence, or absorbance microplate reader can also be used. As described above, in accordance with the reporter assay method of the present invention, it becomes possible to carry out reporter assay as high-throughput assay. Further, automated processing is realized based on robotics. Furthermore, with the use of the reporter assay method of the present invention, convenient quantitative evaluation of interaction between proteins is realized using a two-hybrid system. In addition, development of high-sensitivity bioassay of dioxin, environmental hormones, and the like is achieved at low cost. Moreover, the reporter assay method of the present invention can be used for comprehensive screening of secretory proteins, such screening being useful for drug development. Also, the reporter assay method of the present invention can be used for high-throughput screening for a leading compound for a new drug in a manner such that a yeast in which a human membrane binding receptor, intranuclear receptor, or the like is expressed is used and such receptor and the intracellular signal transduction system of the yeast are subjected to coupling. In addition, gene mutation detection has been carried out by a conventional labor-consuming method wherein a single sample is placed in a single petri dish. However, in accordance with the reporter assay method of the present invention, 96 types of samples can be simultaneously analyzed on a single 96-well plate, which contributes to the realization of high-throughput gene mutation analysis.

Further, the reporter assay method of the present invention involves high-sensitivity reporter assay. Thus, scaling down of reporter assay can be realized. For instance, the transformed yeast of the present invention is set in a chip or capillary in which a channel about 1 μm to 1 mm in length, a membrane filter preventing a yeast from flowing outward, and a narrow channel or a nanopillar are provided such that a medium alone can flow therethrough. Alternatively, a chip or capillary in which a culture solution containing cell bodies is allowed to flow therethrough may be used. Subsequently, a substrate of a secretory luminescent enzyme that has been released into a medium is allowed to flow into the channel such that an enzyme reaction takes place. As described above, the reporter assay method of the present invention can be used for on-chip analysis, which is referred to as liTAS (micro total analysis system), or “Lab-on-a-chip.”

Also, in accordance with the reporter assay method of the present invention described above, secretion capacity of a secretory signal peptide or secretory protein can be evaluated. Specifically, DNA obtained by linking a gene encoding a secretory signal peptide or secretory protein to the upstream of a gene encoding a mature protein of a secretory luminescent enzyme is introduced into a host so that based on the enzyme activity of the secretory luminescent enzyme, the secretion capacity of a secretory signal peptide or secretory protein can be quantitatively evaluated. In such case, a gene encoding a secretory signal peptide or secretory protein is linked to the 5′ end upstream of a gene encoding a mature protein of a secretory luminescent enzyme.

In a case in which a gene encoding a secretory signal peptide is used, the obtained enzyme activity level of a secretory luminescent enzyme can be evaluated as a relative value of the secretion activity of the secretory signal peptide. Alternatively, based on the obtained enzyme activity level, such gene can be evaluated as a gene encoding a secretory signal peptide.

Further, when a gene encoding a secretory protein is used, the obtained enzyme activity level of a secretory luminescent enzyme can be evaluated as a relative value of the secretory protein expression level. Alternatively, based on the obtained enzyme activity level, such gene can be evaluated as a gene encoding a secretory protein.

Furthermore, in accordance with the reporter assay method of the present invention, chemical or physical change can be measured. Herein the term “chemical or physical change” indicates, for example, detection of a chemical substance or a physiologically active substance and concentration change in such substance and temperature change. For instance, when a physiologically active substance such as an environmental hormone or the like is detected or the concentration change in such substance is measured, a promoter that is responsive to such substance or DNA fragment involved in transcriptional control is designated as a foreign DNA fragment. For instance, upon detection of dioxin, a product obtained by linking a usual promoter to a sequence to which a complex of a dioxin receptor and dioxin binds is designated as a foreign DNA fragment. Such foreign DNA fragment is linked to a secretory luminescent enzyme gene. Then, the foreign DNA fragment and a gene unit in which a dioxin receptor is expressed are simultaneously introduced into a host cell. As described above, a reporter assay method for detection of dioxin can be carried out. In such case, the concentration of dioxin can be measured based on the luminescence intensity of a secretory luminescent enzyme. Likewise, also in terms of temperature change, a reporter assay method for detecting temperature change can be established using a protein detecting such change. For instance, high temperature can be detected using a temperature-dependent transcription factor called heat shock factor (HSF) based on the secretory luminescent enzyme activity.

In accordance with the method of the present invention, the promoter that is responsive to chemical or physical change or the DNA fragment involved in transcriptional control described above is designated as a foreign DNA fragment. DNA obtained by linking a secretory luminescent enzyme gene to such foreign DNA fragment is introduced into a host, resulting in the DNA expression. Then, the host is exposed to a subject of chemical or physical change. After the exposure, secretory luminescent enzyme activity is measured. In a case in which the secretory luminescent enzyme activity changes compared with a negative control, it is indicated that the expression of a foreign DNA fragment has been changed in response to a test subject. The thus obtained enzyme activity level of a secretory luminescent enzyme can be evaluated as a relative measurement value based on chemical or physical change. Alternatively, a foreign DNA fragment that interacts with the aforementioned protein that is responsive to chemical or physical change (hereafter to be referred to as “responsive protein”) is introduced into a host, followed by DNA expression. In addition, a secretory luminescent enzyme gene is linked to the foreign DNA fragment. Also, when a responsive protein does not exist in a host, a gene encoding a responsive protein serving as a foreign gene is introduced into a host, followed by gene expression. Thereafter, the host is exposed to a subject of chemical or physical change. After the exposure, the secretory luminescent enzyme activity is measured. In a case in which the secretory luminescent enzyme activity changes compared with a negative control, it is indicated that the responsive protein has detected chemical or physical change so that interaction with the foreign DNA fragment has been changed. As described above, the obtained enzyme activity level of a secretory luminescent enzyme can be evaluated as a relative measurement value based on chemical or physical change.

In accordance with the method of the present invention, as long as the transcriptional level changes, an adequate promoter or a DNA fragment involved in transcriptional control can be used as a foreign DNA fragment. A gene encoding one or more responsive proteins (e.g., a receptor represented by a dioxin receptor) is simultaneously expressed according to need. Alternatively, a method using a signal transduction system of a host is used in combination.

Upon detection or measurement of a chemical substance or a physiologically active substance, a gene encoding an adequate receptor is introduced into a host, followed by intracellular, intranuclear, or cellular membrane expression of the receptor. Also, a gene encoding a protein involved in an adequate signal transduction system is allowed to be expressed in a host according to need such that a detection signal affects the transcriptional activity when such receptor detects a chemical substance or a physiologically active substance. Further, an adequate DNA fragment or a promoter involved in transcriptional control serving as a foreign DNA fragment is linked to a secretory luminescent enzyme gene and the resultant is introduced into a host, followed by the expression thereof. Such receptor, signal transduction factor, or the like may be a host-derived or foreign protein, a fusion protein with another protein, or an artificially produced protein, as long as it can function. In addition, a foreign DNA fragment that is linked to a secretory luminescent enzyme gene has a sequence derived from a host to which the fragment is introduced, a foreign sequence, or an artificially designed sequence, as long as the fragment has functions for transcriptional control.

Hitherto, receptors corresponding to many chemical substances have been known. Examples thereof include a dioxin receptor, an environmental hormone receptor (estrogen receptor), and a peroxisome proliferator-activated receptor (PPAR). That is, the reporter assay method of the present invention can be applied as a method for bioassay of numerous chemical substances or physiologically active substances. Further, many receptors existing on a cellular membrane such as a histamine receptor have been known. Thus, a receptor that has not been specified to bind to a particular substance, or a so-called “orphan receptor,” is combined with the reporter assay method of the present invention. Accordingly, the reporter assay method of the present invention can be used for ligand searches that are highly important in terms of drug development. Upon such ligand searches, a significantly large number of candidate substances must be examined, meaning that high-throughput assay is required. Therefore, the reporter assay method of the present invention is adequate for ligand searches.

Moreover, in accordance with the reporter assay method of the present invention, split assay can be carried out. In such case, a secretory luminescent enzyme is designed to be split at an adequate site thereon into two fragments, and the two fragments (hereafter to be referred to as the “N-terminal fragment” and the “C-terminal fragment”) are actually expressed in a host. Then, the two fragments physically associate with each other in an adequate manner, resulting in the reexpression of the secretory luminescent enzyme activity. Such phenomenon is applied to the present invention such that the association capacity between cell membrane proteins on a cellular membrane is determined (split assay).

In accordance with the method of the present invention, genes encoding two types of cell membrane proteins (hereafter to be referred to as “cell membrane protein gene”) serving as a foreign gene are subjected to association capacity determination. First, expression cassettes of the two types of cell membrane proteins are prepared. The first expression cassette is prepared in a manner such that either one of cell membrane protein genes is linked to a DNA fragment encoding the N-terminal fragment of a secretory luminescent enzyme. More specifically, the first expression cassette contains a fusion gene encoding a fusion protein in which the N-terminal fragment of a secretory luminescent enzyme is linked to the extracellular end of the cell membrane protein. The second expression cassette is prepared in a manner such that the other cell membrane protein gene is linked to a DNA fragment encoding the C-terminal fragment of a secretory luminescent enzyme. As with the case of the first expression cassette, the second expression cassette contains a fusion gene encoding a fusion protein in which the C-terminal fragment of a secretory luminescent enzyme is linked to the extracellular end of the cell membrane protein.

Subsequently, these two types of expression cassettes are introduced into a host such that a fusion gene of each cassette is expressed therein. Then, the secretory luminescent enzyme activity is measured. When the enzyme activity of secretory luminescent enzyme can be obtained, it can be determined that two types of cell membrane proteins associate with each other on a cellular membrane. Meanwhile, when the enzyme activity of secretory luminescent enzyme cannot be obtained, it is suggested that two types of cell membrane proteins do not associate with each other on a cellular membrane.

As described above, in accordance with the reporter assay method of the present invention, the mutual association capacity of two types of cell membrane proteins can be determined. In addition, in accordance with the reporter assay method of the present invention, a gene encoding a cell wall protein is designated as a foreign gene instead of a cell membrane protein gene. Thus, the mutual association capacity of two types of cell wall proteins on a cell wall can be determined.

### EXAMPLES

The present invention will be hereafter described in greater detail with reference to the following examples, although the technical scope of the present invention is not limited thereto.

### Example 1

### Examination of the Secretory Expression of Cypridina-noctiluca-secretory Luciferase in Yeasts and the Optimum pH of the Culture Solution Thereof

(1) Production of a Transformant (Transformed Yeast) Containing Cypridina noctiluca Secretory Luciferase

With the use of cDNA (SEQ ID NO: 1) encoding Cypridina noctiluca-secretory luciferase (the amino acid sequence set forth in SEQ ID NO: 2; hereafter to be referred to as “CLuc”), the secretory expression of the secretory luciferase was induced in budding yeasts (Saccharomyces cerevisiae). In addition, the pH condition of a medium used for the secretory expression of CLuc was examined.

First, a plasmid containing CLuc cDNA (hereafter to be referred to as “pcDNA-CL”) was used to amplify cDNA encoding a mature protein (hereafter to be referred to as “mature CLuc cDNA”) obtained by removing a CLuc secretory signal peptide (1^{st} to 18^{th} amino acids in the amino acid sequence of CLuc set forth in SEQ ID NO: 2) from pcDNA-CL by polymerase chain reaction (PCR).

Meanwhile, DNA encoding a secretory signal peptide (the amino acid sequence set forth in SEQ ID NO: 12) of an α-factor of a budding yeast (SEQ ID NO: 11; hereafter to be referred to as “α-factor secretory signal peptide DNA”) was amplified using Saccharomyces cerevisiae S288C genomic DNA (purchased from Invitrogen) as a template.

Then, mature CLuc cDNA and the α-factor secretory signal peptide DNA were linked to each other so as to be subjected to overlap PCR such that a gene (SEQ ID NO: 13) that encodes mature CLuc having an α-factor secretory signal peptide at the N terminal (the amino acid sequence set forth in SEQ ID NO: 14; hereafter to be referred to as “αCLuc”) was produced.

Primer sequences described below were used to amplify mature CLuc cDNA (alpha-luci-F and 3′Xba I-luci).

alpha-luci-F:

(SEQ ID NO: 16)

GAAAAGAGAGGCTGAAGCTCAGGACTGTCCTTACGAACC
3′Xba I-luci:

(SEQ ID NO: 17)

CCCTGTCTAGACTATTTGCATTCATCTGGTAC.

Alpha-luci-F has a sequence in which a 20-bp downstream sequence comprising the first codon (corresponding to the 19^{th} amino acid in the amino acid sequence of CLuc set forth in SEQ ID NO: 2) of mature CLuc is linked to the downstream of a 19-bp DNA sequence starting from the 3′ end of DNA encoding an α-factor secretory signal peptide. In addition, 3′Xba I-luci has a sequence complementary to a 21-bp upstream sequence comprising the termination codon of CLuc.

PCR was carried out to amplify mature CLuc cDNA using 50 μl of a reaction solution containing 300 nM each of the primers, 200 μM of dNTP (a mixed solution of 4 types of deoxynucleotide triphosphates), 100 μM of MgSO₄, 10 ng of pcDNA-CL serving as a template, KOD Plus buffer (1×), and KOD plus DNA polymerase (1 U) by the following steps: a first step at 94° C. for 2 minutes; a second step at 94° C. for 15 seconds (denaturation), 48° C. for 30 seconds (annealing), and 68° C. for 2 minutes (elongation) for 35 cycles; and a third step at 68° C. for 3 minutes.

The obtained PCR product was analyzed by 1% agarose electrophoresis. Accordingly, a DNA fragment (of approximately 1.6 kb) containing mature CLuc cDNA was confirmed. Hereafter, this DNA fragment is referred to as “DNA fragment A.”

Meanwhile, the following primer sequences (5′Sma I-alpha and alpha-luci-R) were used to amplify an α-factor secretory signal peptide DNA of a budding yeast:

5′Sma I-alpha:

(SEQ ID NO: 18)

GGGTCCCGGGATGAGATTTCCTTCAATTT;
and
alpha-luci-R:

(SEQ ID NO: 19)

GGTTCGTAAGGACAGTCCTGAGCTTCAGCCTCTCTTTTC.

5′Sma I-alpha is a 28-bp downstream sequence comprising the initiation codon (ATG) of an α-factor secretory signal peptide. Alpha-luci-R is a sequence complementary to the above alpha-luci-F (SEQ ID NO: 16).

PCR was carried out to amplify α-factor secretory signal peptide DNA under basically the same conditions used for the above mature CLuc cDNA amplification except that: Saccharomyces cerevisiae S288C genomic DNA (1 ng) was used as a template; 5′Sma I-alpha and alpha-luci-R were used as primers; the elongation reaction time was 30 seconds in a second step; and a third step was carried out for 1 minute.

The obtained PCR product was analyzed by 1% agarose electrophoresis. Accordingly, a DNA fragment (of approximately 250 bp) containing cc-factor secretory signal peptide DNA was confirmed. Hereafter, this DNA fragment is referred to as “DNA fragment B.”

Overlap PCR was carried out to link the DNA fragment A to the downstream of the DNA fragment B using primers 5′Sma I-alpha (SEQ ID NO: 18) and 3′Xba I-luci (SEQ ID NO: 17).

Overlap PCR was carried out under basically the same conditions used for the above mature CLuc cDNA amplification except that: 1 μl each of a PCR reaction solution containing the DNA fragment A and a PCR reaction solution containing the DNA fragment B, each of which had been diluted 100-fold with distilled water, was used as a template; 5′Sma I-alpha (SEQ ID NO: 18) and 3′Xba I-luci (SEQ ID NO: 17) were used as primers; the annealing temperature was 50° C. and the elongation reaction time was 2 minutes in a second step; and a third step was carried out for 3 minutes.

The obtained PCR product was purified using a GenElute PCR clean-up kit (Sigma). Then, DNA was eluted from the column of the kit using 40 μl of distilled water. The DNA was cleaved with SmaI (20 U) in 50 μl of a reaction solution for 18 hours. After being cleaved with SmaI, DNA was purified again using a GenElute PCR clean-up kit. Then, DNA was eluted from the column of the kit using 40 μl of distilled water.

Next, the eluted DNA was cleaved with XbaI (20 U) in 50 μl of a reaction solution for 18 hours. After being cleaved with XbaI, DNA was purified using a GenElute PCR clean-up kit. Then, DNA was eluted from the column of the kit using 40 μl of distilled water. Accordingly, a DNA fragment in which mature CLuc cDNA was linked to the downstream of α-factor secretory signal peptide DNA was obtained. Hereafter, the DNA fragment is referred to as DNA fragment C.

Meanwhile, pUG35 (http://mips.gsf.de/proj/yeast/info/tools/hegemann/gfp.html) was cleaved with XbaI and Sacd, followed by blunting with T4 DNA polymerase. Then, self circularization of the resultant was induced such that pUG35-MET25+MCS was prepared. Next, pUG35-MET25+MCS was cleaved with ClaI and XhoI, followed by agarose electrophoresis. Thus, a vector fragment (of approximately 5.1 kbp) was recovered. On the other hand, in order to cause circularization of the vector fragment, oligo DNAs described below were synthesized, followed by annealing. Thus, linker DNA was prepared.

(SEQ ID NO: 20)

| | |
|---|---|
| MCS linker F: | CCGCTCGAGCGGCCGCGAGCTCGTCGACATCGATGG |

(SEQ ID NO: 21)

| | |
|---|---|
| MCS linker R: | CCATCGATGTCGACGAGCTCGCGGCCGCTCGAGCGG |

In addition, the prepared linker DNA contained restriction enzyme sites of XhoI-NotI-SacI-SalI-ClaI.

After annealing, both ends of the linker DNA were cleaved with XhoI and ClaI. Then, the vector fragment and the linker DNA were linked to each other using a DNA Ligation Kit ver. 2. The resultant was introduced into Escherichia coli DH5α. The obtained transformant was cultured overnight. Then, a plasmid was extracted therefrom using a QuantumPrep Plasmid MiniPrep Kit. Based on a restriction enzyme cleavage pattern and sequence analysis, a transformant comprising the plasmid of interest was identified. The plasmid of the interest (hereafter to be referred to as “pUG35-MET25-EGFP3+MCS”) was prepared from the transformant.

Further, the pUG35-MET25-EGFP3+MCS plasmid (2 μg) was cleaved with SmaI (50 U) in 50 μl of a reaction solution for 18 hours. After being cleaved with SmaI, DNA was purified using a GenElute PCR clean-up kit. Then, DNA was eluted from the column of the kit using 40 μl of distilled water. Subsequently, the obtained DNA was cleaved with XbaI (50 U) in 50 μl of a reaction solution for 18 hours. After being cleaved with XbaI, DNA was subjected to agarose electrophoresis. Accordingly, a vector fragment of the pUG35-MET25-EGFP3+MCS plasmid (of approximately 5 kb) was recovered. Hereafter, the vector fragment is referred to as DNA fragment D.

The DNA fragment C and the DNA fragment D were linked to each other using a DNA Ligation Kit ver. 2.1 (TAKARA BIO INC.) so as to be circularized. Then the resultant was introduced into Escherichia coli DH5α. The obtained transformant was cultured overnight. Then, a plasmid was extracted therefrom using a GenElute Plasmid MiniPrep kit (Sigma). In addition, based on SpeI and XbaI cleavage patterns of the plasmid that was extracted, a transformant comprising a plasmid into which. DNA encoding αCLuc had been inserted was identified.

In addition, the obtained plasmid solution was subjected to nucleotide sequence analysis using a BigDye Terminator Cycle Sequencing Ready Reaction kit ver. 3.1 (Applied Biosystems). Accordingly, the plasmid was confirmed to have a nucleotide sequence that was identical to an expected nucleotide sequence encoding αCLuc. Thus, the plasmid (hereafter to be referred to as “pCLuRA”) was prepared from a transformant from which a plasmid having a nucleotide sequence encoding αCLuc is derived.

Next, a promoter (SEQ ID NO: 22; hereafter to be referred to as “TDH3 promoter”) of the Saccharomyces cerevisiae TDH3 gene (systematic gene name: YGR192C) was incorporated into pCLuRA in a manner such that the promoter was located at the 5′ upstream region of DNA encoding αCLuc. Herein, the TDH3 promoter indicates an untranslated region in the 5′ upstream region of a TDH3 gene; that is to say, a region sandwiched by the TDH3 gene and the PDX1 gene that is the 5′ upstream adjacent gene of the TDH3 gene (see the Yeast Genome Database: http://www.yeastgenome.org/).

PCR was carried out to isolate the TDH3 promoter using primer sequences described below (5′TDH3_BamHI and 3′TDH3):

| | |
|---|---|
| 5′TDH3_BamHI: | |
| GGGTGGATCCCGAGTTTATCATTATCAATAC | (SEQ ID NO: 23) |
| 3′TDH3: | |
| TCGAAACTAAGTTCTTGGTG | (SEQ ID NO: 24) |

The 5′TDH3_BamH1 primer has a sequence in which a BamHI restriction enzyme site is added to the 5′ region of 21 bases 3′ downstream of the termination codon of PDX1 ORF 5′ upstream of the TDH3 open reading frame (ORF). The 3′TDH3 primer is a sequence complementary to 20 bases 5′ upstream starting from TDH3 ORF initiation codon. In. addition, see the Yeast Genome Database (http://www.yeastgenome.org/) regarding the positions of the primers.

PCR was carried out under basically the same conditions used for the above mature CLuc cDNA amplification except that: Saccharomyces cerevisiae S288C genomic DNA (1 ng) was used as a template and 5′TDH3_BamHI (SEQ ID NO: 23) and 3′TDH3 (SEQ ID NO: 24) were used as primers; the annealing temperature was 52° C. and the elongation reaction time was 30 seconds in a second step; and a third step was carried out for 1 minute.

The obtained PCR product was analyzed by 1% agarose electrophoresis. Accordingly, a DNA fragment (of approximately 650 bp) was confirmed. Thus, the obtained PCR product was purified using a GenElute PCR clean-up kit. Then, DNA was eluted from the column of the kit using 40 μl of distilled water. The thus obtained DNA fragment of the TDH3 promoter is hereafter referred to as “DNA fragment E.”

Meanwhile, pCLuRA (2 μg) was cleaved with SmaI (50 U) for in 50 μl of a reaction solution for 18 hours. After being cleaved with SmaI, DNA was purified using a GenElute PCR clean-up kit. Then, DNA was eluted from the column of the kit using 40 μl of distilled water. Subsequently, the obtained DNA was cleaved with BamHI (50 U) in 50 μl of a reaction solution for 18 hours, followed by agarose electrophoresis. Then a vector fragment (of approximately 5 kb) was recovered. Hereafter the vector fragment is referred to as “DNA fragment F.”

The DNA fragment E and the DNA fragment F were linked to each other using a DNA Ligation Kit ver. 2 so as to be circularized. The resultant was introduced into Escherichia coli DH5α. The obtained transformant was cultured overnight. A plasmid was extracted therefrom using a GenElute Plasmid MiniPrep kit. In addition, based on a restriction enzyme cleavage pattern of the extracted plasmid and nucleotide sequence analysis, a transformant comprising a plasmid into which DNA encoding a TDH3 promoter had been inserted was identified. A plasmid (hereafter to be referred to as “pCLuRA-TDH3”) was prepared from a transformant from which the plasmid into which DNA encoding a TDH3 promoter had been inserted was derived.

With the use of pCLuRA-TDH3, Saccharomyces cerevisiae strain YPH500 was transformed. An EZ-transformation kit (BIO101) was used for transformation.

The obtained transformant was applied to a uracil-free synthetic agar medium (a SD+KHLWade plate; 0.67% yeast nitrogen base without amino acids (DIFCO), 2% glucose, 0.02 mg/ml adenine sulfate, 0.02 mg/ml tryptophan, 0.02 mg/ml histidine, 0.03 mg/ml lysine, 0.03 mg/ml leucine, and 1.5% agar), followed by culture at 30° C. for 3 days.

After culture for 3 days, a transformant comprising pCLuRA-TDH3 was obtained. In the transformant comprising pCLuRA-TDH3, αCLuc is expressed.

(2) Examination of the Secretory Expression of Cypridina noctiluca-secretory Luciferase From a Transformant and the Optimum pH of the Culture Solution Thereof

The transformant comprising pCLuRA-TDH3 obtained above was introduced into a uracil-free synthetic medium (SD+KHLWade; 0.67% yeast nitrogen base without amino acids (DIFCO), 2% glucose, 0.02 mg/ml adenine sulfate, 0.02 mg/ml tryptophan, 0.02 mg/ml histidine, 0.03 mg/ml lysine, and 0.03 mg/ml leucine; hereafter to be simply referred to as “SD medium”), followed by culture at 30° C. In addition, SD media containing 100 mM potassium phosphate buffer solutions that had been adjusted to different pH levels (pH 4.0, 5.0, 6.0, and 6.5) were prepared. As described above, the transformant comprising pCLuRA-TDH3 was introduced into the media, followed by culture at 30° C.

After culture, when absorbance at 600 nm reached 0.4 to 0.6, a portion of each culture solution was collected and cell bodies were removed therefrom by centrifugation. Thus, the culture supernatants were prepared. The culture supernatants were supposed to contain CLuc that had been secreted from the transformant comprising pCLuRA-TDH3. Therefore, the CLuc activity in each culture supernatant was measured.

For the CLuc activity measurement, an LB960 luminometer (Berthold) was used. Cypridina luciferin (a substrate of. luciferase) used for luminescence measurement was prepared in a manner such that a preservative solution (66 or 100 μM) dissolved in a 50% ethanol-5 mM HCl solution was diluted to 2.5 μM with 100 mM Tris-HCl (pH 7.4) when used.

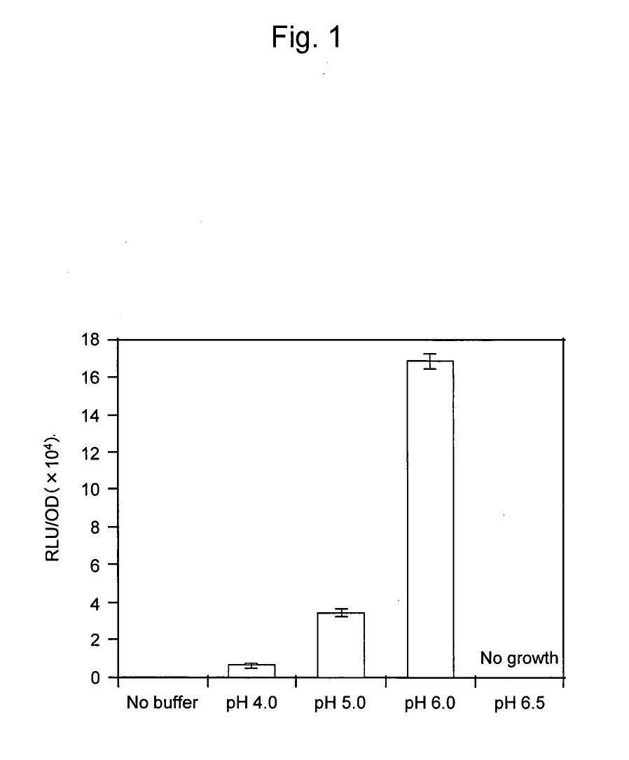

The luminescence intensity upon degradation of Cypridina luciferin by CLuc was measured based on incorporation for 5 seconds by adding 80 μl of a 2.5 μM Cypridina luciferin diluted solution to 20 μl of the culture supernatant prepared above. The measurement values were divided by absorbance of the culture solution at 600 nm such that CLuc activity subjected to a correction of the number of yeast cell bodies was obtained. FIG. 1 shows the results.

In FIG. 1, “RLU/OD” on the vertical axis indicates a value obtained by dividing relative light units from a luminometer by absorbance at 600 nm. In addition, terms on the horizontal axis stand for the following samples:

“no buffer solution:” sample from an SD medium to which a 100 mM potassium phosphate buffer solution had not been added and in which the transformant comprising pCLuRA-TDH3 was cultured; and

“pH 4.0,” “pH 5.0,” “pH 6.0,” and “pH 6.5:” samples from SD media containing 100 mM potassium phosphate buffer solutions that had been adjusted to pH 4.0, 5.0, 6.0, and 6.5, respectively, and in each of which the transformant comprising pCLuRA-TDH3 was cultured.

Further, in FIG. 1, the RLU/OD value of each sample is represented by the mean value and the standard deviation.

As shown in FIG. 1, in the case of the ordinary SD medium to which a buffer solution (100 mM potassium phosphate buffer solution) had not been added and the pH level of which gradually declined, the transformant comprising pCLuRA-TDH3 proliferated; however, the CLuc activity was not detected in the culture supernatant. In addition, in the case of the SD medium containing a buffer solution that had been adjusted to pH 6.5, the transformant comprising pCLuRA-TDH3 was not grown or did not proliferate. Meanwhile, in the cases of the SD medium to which a buffer solution at pH 4.0, 5.0, or 6.0 had been added such that the pH of the medium did not change, it was possible to detect the CLuc activity in the culture supernatant. In particular, in the case of the SD medium to which the buffer solution at pH 6.0 had been added (i.e., the medium with pH 6.0), the maximum and optimum value of the CLuc activity in the culture supernatant was obtained. In addition, the CLuc activity measurement using these culture supernatant samples was carried out at pH 7.4, at which the maximum value of CLuc activity was obtained. As described above, culture conditions such that a transformant (transformed yeast) is able to proliferate and CLuc is not deactivated have been established.

### Example 2

### Examination of pH and Salt Concentration Upon CLuc Activity Measurement

The conditions of pH and salt concentration upon CLuc activity measurement following the secretory expression of CLuc in yeasts were examined.

A culture supernatant containing αCLuc secreted from the transformant comprising pCLuRA-TDH3 that had been prepared in Example 1 was prepared as with the case of Example 1. The pH of medium was determined to be 6.0.

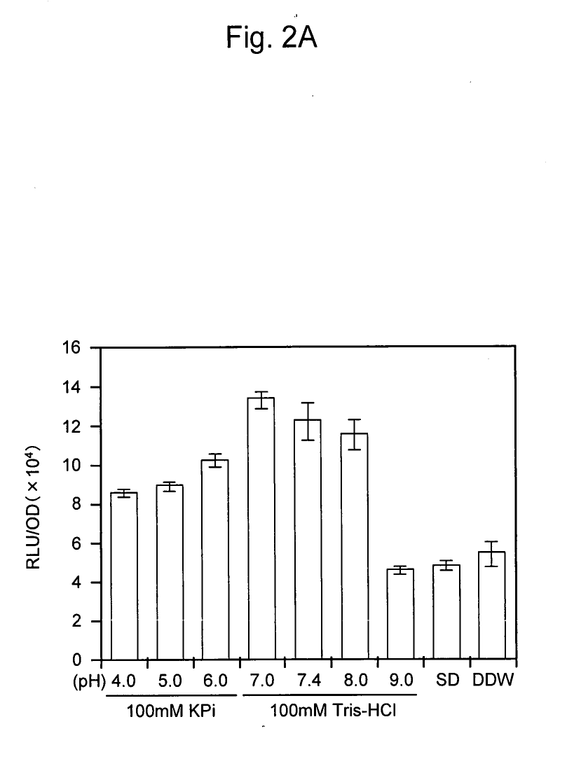

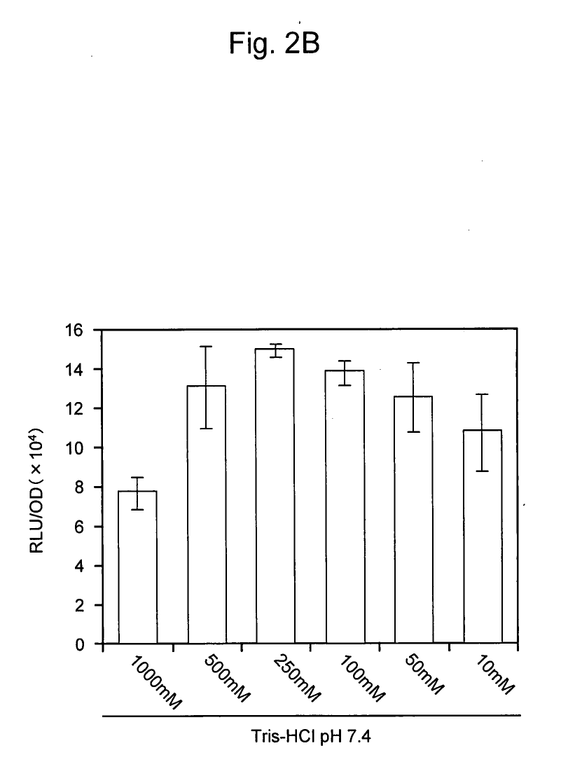

In order to set the pH upon CLuc activity measurement, Cypridina luciferin was diluted with buffer solutions at different pH levels (potassium phosphate buffer solutions (KPi) at final concentrations of 100 mM or Tris-hydrochloric acid buffer solutions (Tris-HCl)) such that the obtained diluted solutions (2.5 μM) were used. In addition, each Cypridina luciferin diluted solution was added to the culture supernatant in a volume 4 times that of the culture supernatant. CLuc activity measurement was carried out as with the case of Example 1. FIG. 2A shows the results.

Alternatively, Cypridina luciferin diluted solutions (2.5 μm) that had been diluted with Tris-HCl (pH 7.4) solutions at different final concentrations were separately added to the culture supernatant in a volume 4 times that of the culture supernatant. Then, CLuc activity measurement was carried out as with the case of Example 1. FIG. 2B shows the results.

In FIGS. 2A and 2B, “RLU/OD” on the vertical axis indicates a value obtained by dividing relative light units from a luminometer by absorbance at 600 nm. The RLU/OD value of each sample is represented by the mean value and the standard deviation. In addition, in FIG. 2A, “SD” stands for a sample for which a Cypridina luciferin diluted solution that had been diluted with an SD medium not containing a buffer solution was used. Also, “DDW” stands for a sample for which a Cypridina luciferin diluted solution that had been diluted with water was used.

As is apparent from FIGS. 2A and 2B, the CLuc (αCLuc) activity was found to change depending on the salt concentration and pH of the luciferin diluted solution upon activity measurement. When the luciferin diluted solution that had been diluted with 100 mM Tris-HCl (pH 7.0 to 8.0) was used, the maximum level of CLuc activity was obtained.

### Example 3

### Examination of Luciferin Concentration Upon CLuc Activity Measurement

Luciferin concentration upon CLuc activity measurement following the secretory expression of CLuc in yeasts was examined.

A culture supernatant comprising aCLuc secreted from the transformant comprising pCLuRA-TDH3 that had been prepared in Example 1 was prepared as with the case of Example 1. The pH of the medium was determined to be 6.0. Cypridina luciferin used was diluted with 100 mM Tris-HCl solutions (pH 7.4) at different concentrations. That is, Cypridina luciferin was diluted so as to have final concentrations of 0.25, 0.5, 1.0, 2.0, and 4.0 μM in reaction solutions. Also, the Cypridina luciferin diluted solutions with different concentrations were adjusted to have the same concentration of ethanol used for dissolution upon the obtaining of preservative solutions.

In addition, a stock solution of the culture supernatant of the transformant comprising pCLuRA-TDH3 was separately serial-diluted 10-fold, 100-fold, 1000-fold, and 10000-fold with an SD medium containing a 100 mM phosphate buffer solution so that the resulting diluted culture supernatants were used. Specifically, the concentration of the culture supernatant stock solution was determined to be 100% such that 10-fold, 100-fold, 1000-fold, and 10000-fold diluted culture supernatants had supernatant concentrations of 10%, 1%, 0.1%, and 0.01%, respectively.

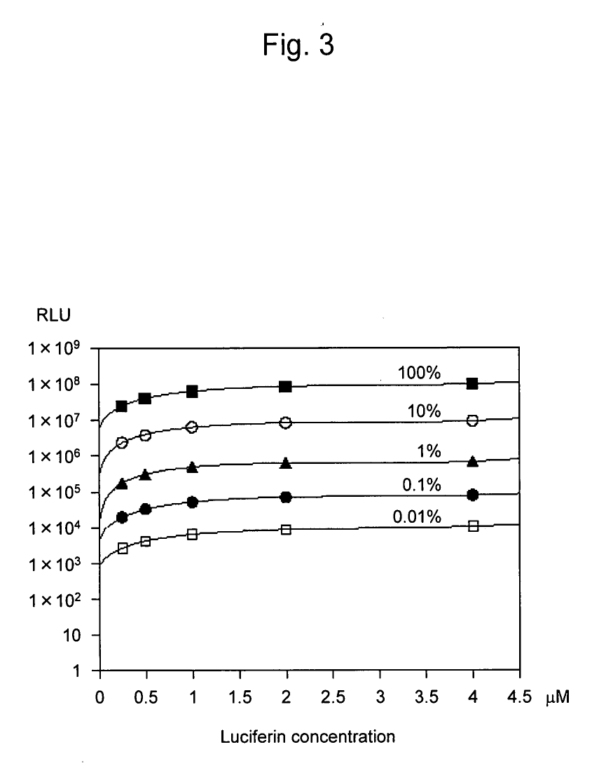

The different Cypridina luciferin diluted solutions were separately added to each culture supernatant prepared above in a volume 4 times that of the culture supernatant. Then, CLuc activity measurement was carried out as with the case of Example 1. FIG. 3 shows the results.

In FIG. 3, plots indicate results obtained by using culture supernatants that had been serial-diluted with a culture supernatant stock solution (100%) with reaction solutions with different final concentrations of luciferin. “RLU” on the vertical axis stands for the relative light units from a luminometer.

As shown in FIG. 3, concentration-reaction curves of the CLuc activity corresponding to the Michaelis-Menten equation were obtained. The activity was found to reach a saturation point when the final luciferin concentration in a reaction solution was approximately 1 μM or more.

Thus, in the following examples, CLuc activity measurement was carried out with the addition of luciferin such that the final luciferin concentration in a reaction solution was 2 μM.

### Example 4

### Examination of the Stability of CLuc in Yeast Culture Supernatants

The stability of CLuc in yeast culture supernatants following the secretory expression of CLuc in yeasts was examined.

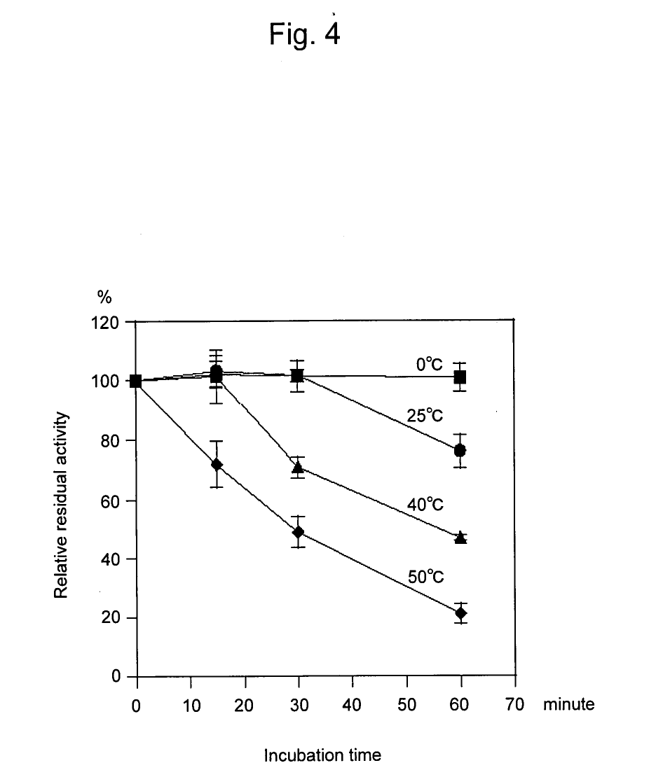

Preparation of a culture supernatant comprising αCLuc secreted from the transformant comprising pCLuRA-TDH3 that had been prepared in Example 1 and CLuc activity measurement were carried out as with the case of Example 1. The prepared culture supernatants were incubated at 0° C., 25° C., 40° C., and 50° C. for 0, 15, 30, and 60 minutes, respectively, followed by CLuc activity measurement. FIG. 4 shows the results.

In FIG. 4, each plot indicates the mean value and the standard deviation of relative residual activities of each sample subjected to incubation at a given temperature for a given period of time. The vertical axis denotes the relative residual activity based on the CLuc activity (100%) of a sample that had not been incubated.

As is shown in FIG. 4, CLuc was highly stable as an enzyme. CLuc was proved to be stable at room temperature even 15 minutes after the sampling that is required for the high-throughput assay method of the present invention.

### Example 5

### Examination of the Measurable Range Upon CLuc Activity Measurement

The measurable range (dynamic range) of the CLuc activity in yeast culture supernatants following the secretory expression of CLuc in yeasts was examined.

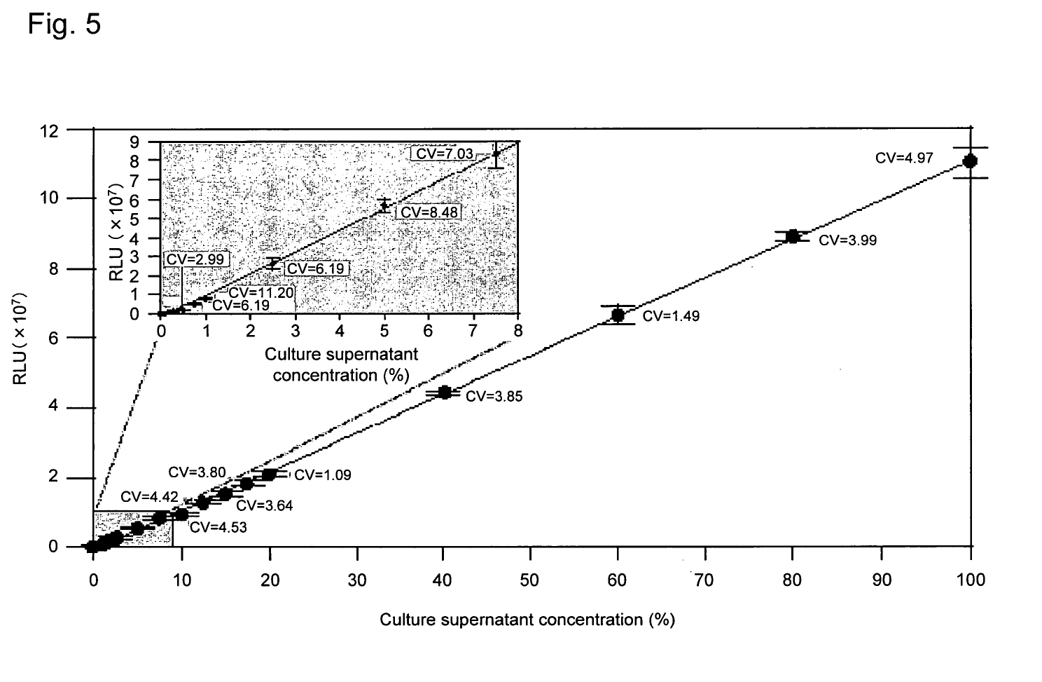

Preparation of a culture supernatant comprising αCLuc secreted from the transformant comprising pCLuRA-TDH3 that had been prepared in Example 1 and CLuc activity measurement were carried out as with the case of Example 1. In addition, the prepared culture supernatant was serial-diluted up to 1000-fold with a SD medium containing a 100 mM phosphate buffer solution such that the diluted culture supernatants were used for CLuc activity measurement. Specifically, the concentration of a culture supernatant stock solution was determined to be 100% such that the most diluted culture supernatant that was prepared had a culture supernatant concentration of 0.01%. FIG. 5 shows the results.

In FIG. 5, each plot indicates the mean value and the standard deviation of RLUs of serial-diluted culture supernatant samples (culture supernatant concentration: %). “RLU” on the vertical axis stands for the relative light units from a luminometer. In addition, CV values shown in the figure indicate variation coefficients (standard deviation/mean value×100) obtained through three repeated measurements.

As shown in FIG. 5, it was found that linearity was obtained in terms of CLuc activity within the range of 10³. Accordingly, it has been proved that changes (up to 1000-fold) can be quantified in the reporter assay system using CLuc.

### Example 6

### Examination of Influences of Various Chemical Substances on CLuc Activity Measurement

Influences of chemical substances on CLuc activity measurement were examined, as such substances coexist with yeast culture supernatants upon CLuc activity measurement following the secretory expression of CLuc in yeasts.

Various types of chemical substances (DTT, CuSO₄, Menadione, diamide, H₂O₂, ethanol, NaCl, sorbitol, galactose, raffinose, sucrose, and mannose) used for the examination have been known to induce yeast gene expression (Audrey P. Gasch, Paul T. Spellman, Camilla M. Kao Orna Carmel-Harel, Michael B. Eisen, Gisela Storz, David Botstein, and Patrick O. Brown (2000) Mol. Biol. Cell. 11, 4241-4257; Varela J. C., Praekelt U. M., Meacock P. A., Planta R. J., Mager W. H., (1995) Mol Cell Biol. 15: 6232-45; Macreadie I. G., Horaitis O., Verkuylen A. J., Savin K. W. (1991) Gene. 104: 107-11). In this Example, various types of chemical substances were serial-diluted from a known concentration at which the yeast gene expression can be sufficiently induced.

Preparation of a culture supernatant comprising αCLuc secreted from the transformant comprising pCLuRA-TDH3 that had been prepared in Example 1 and CLuc activity measurement were carried out as with the case of Example 1.

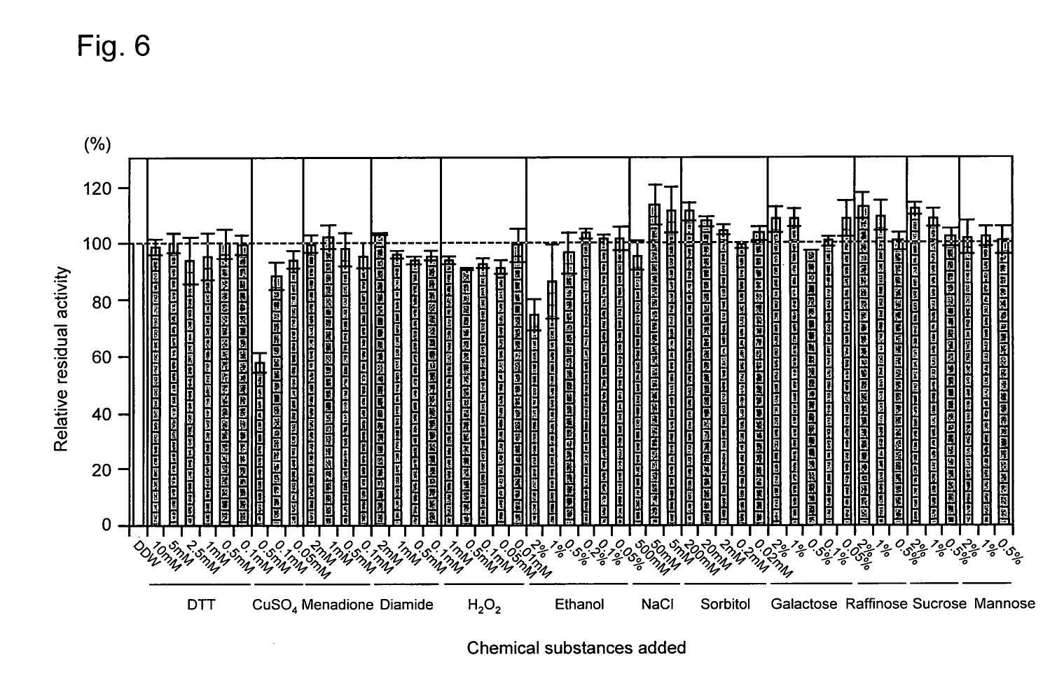

Chemical substances (2 μl) that had been adjusted to have different concentrations were separately added to 20 μl of the prepared culture supernatant, followed by CLuc activity measurement. FIG. 6 shows the results.

FIG. 6 shows relative residual activities with respect to chemical substances that had been separately added to the culture supernatant at different concentrations. The relative residual activity of each sample is represented by the mean value and the standard deviation on the condition that the CLuc activity of a sample to which a chemical substance was not added is determined to be 100%.

As shown in FIG. 6, it has been revealed that the enzyme activity of CLuc is not inhibited by chemical substances that activate yeast gene expression, excluding highly concentrated CuSO₄ or ethanol. Accordingly, it has been proved that reporter assay system using CLuc as a reporter protein can be applied to a wide range of chemical substances.

### Example 7

### Examination of a Method for Correcting the Amount of CLuc Produced Based on Turbidity

The amount of CLuc produced depends on the promoter activity in yeasts and the number of yeast cells. Thus, in order to measure the strength of a yeast promoter based on the CLuc activity, it is necessary to correct the number of yeast cells. Therefore, culture solutions at different cell body densities of a single type of transformed yeast were prepared, followed by CLuc activity measurement. In addition, it was examined whether or not the number of yeast cells were able to be corrected based on turbidity (absorbance at 600 nm) as an index of cell body density.

The transformant comprising pCLuRA-TDH3 that had been prepared in Example 1 was cultured as with the case of Example 1. Culture was carried out at 30° C. for approximately 48 hours. At the stationary phase, a culture solution was obtained. The culture solution was diluted with a new medium such that the concentration thereof was changed from 1/100 to 1/1000. Then, culture was continued at 30° C. for approximately 20 hours.

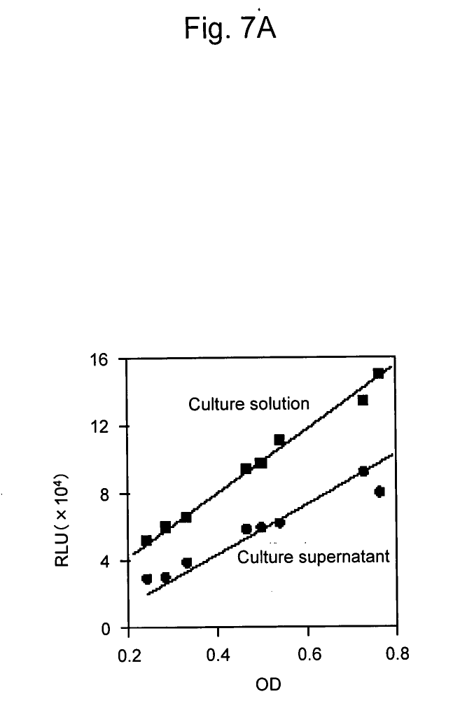

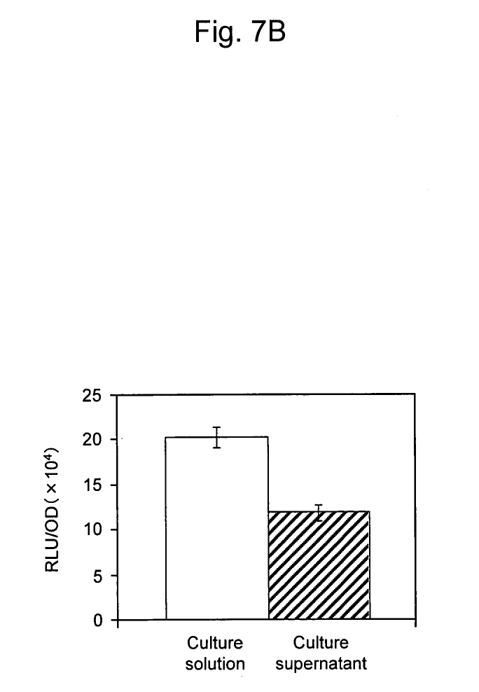

After culture, preparation of a culture supernatant and CLuc activity measurement were carried out as with the case of Example 1. In addition, 200 μl of the culture solution or culture supernatant was collected. The turbidity of the culture solution or culture supernatant was measured using a Tecan Sunrise Remote with absorbance at 600 nm (OD). FIGS. 7A and 7B show the results.

FIG. 7A shows relative luminescence concentrations in culture solutions or culture supernatants at different turbidities. “RLU” on the vertical axis stands for the relative luminescence concentration from a luminometer.

Meanwhile, FIG. 7B shows the mean value and the standard deviation (RLU/OD) obtained from all samples, including the culture solutions containing cell bodies and the culture supernatants obtained by centrifugation of the culture solutions shown in FIG. 7A. “RLU/OD” on the vertical axis indicates a value obtained by dividing the relative light units from a luminometer by absorbance at 600 nm.

As shown in FIG. 7B, the RLU of a culture solution or culture supernatant was corrected with turbidity of a culture solution such that the almost equivalent results shown as RLU/OD were obtained from culture solutions or culture supernatants of cultured yeasts comprising the identical promoter (TDH3 promoter). That is, it has been demonstrated that promoter activity can be evaluated by the method of the present invention even with the use of yeasts in different growth phases without controlling yeasts being in the same growth phase. Further, it has been found that there is a good correlation between measurement results obtained from culture supernatants and measurement results obtained from culture solutions containing cell bodies. Thus, it has been proved that centrifugation of cell bodies is not necessary for the measurement system.

### Example 8

### The stability of CLuc Activity After Sampling of Culture Solutions

In order to carry out high-throughput assay using a 96-well format by the method of the present invention, it is required that the CLuc activity level does not change while 96 samples are being subjected to CLuc activity measurement using a luminometer after sampling of culture solutions.

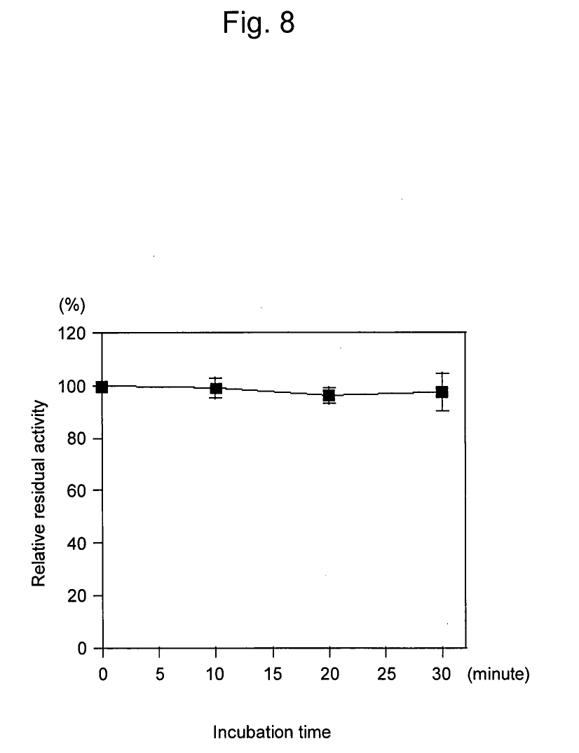

Thus, in order to examine the stability of CLuc activity after sampling of culture solutions, a transformant comprising pCLuRA-TDH3 prepared in Example 1 was cultured as with the case of Example 1. Then, the yeast culture solution was partially collected (sampling), followed by incubation at 25° C. for 0, 10, 20, and 30 minutes. After incubation, the CLuc activity of each culture solution sample was measured as with the case of Example 1. FIG. 8 shows the results.

In FIG. 8, CLuc activity is shown as the relative residual activity based on the CLuc activity of a sample that was measured immediately after sampling (100%). FIG. 8 shows the mean value and the standard deviation of the relative residual activities of all the samples.

As shown in FIG. 8, after sampling, there was almost no change in CLuc activity, even after incubation of the culture solution at 25° C. for 30 minutes. Specifically, there was almost no change in CLuc activity in the culture solution even after it was allowed to stand at room temperature for a time period of 30 minutes after sampling, such period being necessary for measurement of 96 samples using a luminometer. Thus, it has been demonstrated that high throughput measurement can be achieved with the use of a reporter assay system in accordance with the method of the present invention.

### Example 9

### Establishment of a High Throughput Reporter Assay Method Wherein Simultaneous Culture of CLuc-expressing Yeasts is Carried Out With the Use of a 96-deep Well Plate

In order to apply high-throughput assay to the method of the present invention, it is necessary to culture transformed yeasts in a 96-well format. Thus, the same transformed yeasts were separately cultured in a 96-deep well plate so as to obtain 96 samples. Then, it was examined whether or not the CLuc activities corrected with the turbidities exhibited by the samples corresponded to one another.

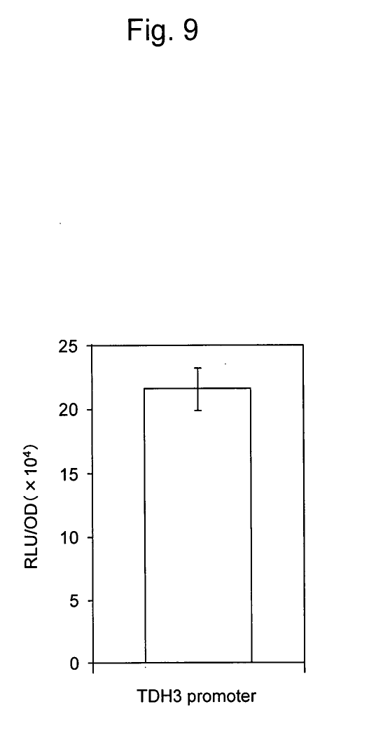

The transformant comprising pCLuRA-TDH3 prepared in Example 1 was cultured at 30° C. as with the case of Example 1. A culture solution that had reached the stationary phase was obtained. The culture solution was diluted with a new medium to a concentration of 1/100. 1 ml of the culture solution was introduced into each well of a 96-deep well plate. Then, the culture was continued at 30° C. for approximately 16 hours. The yeast culture solution was partially collected from each well, followed by incubation at 25° C. for 0, 10, 20, and 30 minutes. Then, the CLuc activity of each culture solution sample was measured as with the case of Example 1. FIG. 9 shows the results.

FIG. 9 shows the mean value and the standard deviation (RLU/OD) when culture was independently carried out in a 96-deep well plate, followed by CLuc activity measurement. In FIG. 9, “RLU/OD” on the vertical axis stands for the relative light units from a luminometer divided by absorbance at 600 nm.

As shown in FIG. 9, with the use of a 96-deep well plate for culture, the CLuc activity level corrected with turbidity was almost the same upon simultaneous culture of many samples. Thus, it has been demonstrated that high throughput measurement can be achieved with the use of the reporter assay system in accordance with the method of the present invention.

### Example 10

### Preparation of DNA Encoding CLuc in Which the Nucleotide Sequence has been Modified so as to be Used in Yeasts

In order to use CLuc as a reporter enzyme in budding yeasts (Saccharomyces cerevisiae), DNA encoding CLuc was redesigned. Firstly, the amino acid sequence of CLuc was converted into a nucleotide sequence using optimum codon of a yeast with reference to the frequency of using codon in a yeast described in the paper of Akashi et al. (Genetics 164: 1291-1303 (2003)). Then, a cis sequence contained in such nucleotide sequence encoding the specific CLuc described above and to which a yeast transcription factor may bind was searched for on a web site (Yeast Promoter Database; SCPD (http://cgsigma.cshl.org/jian/)). Such cis sequence found in the above search, to which a yeast transcription factor may bind, was subjected to substitution of 1 or more bases such that the amino acid sequence encoded by the sequence remained unchanged. Search for potential cis sequences was carried out in a similar manner with the use of a web site for control region analysis (http://www.genomatix.de/) (provided by Genomatix). These two types of cis sequence databases were repeatedly used so that searches for potential cis sequences and removal of such sequences by base substitution were repeatedly performed. Eventually, the sequence of DNA encoding CLuc with the fewest potential transcription factor binding sequences was designed (SEQ ID NO: 15). Hereafter, such sequence is referred to as “mCLuc DNA.” The nucleotide sequence of mCLuc DNA differs from that of CLuc cDNA (SEQ ID NO: 1). However, the amino acid sequence of the protein encoded by mCLuc DNA is identical to that of CLuc (SEQ ID NO: 2). Note that a protein encoded by mCLuc DNA is hereafter referred to as “mCLuc.”

(1) Production of mCLuc DNA Fragments

Whole DNA sequences of double strands constituting mCLuc DNA were produced by custom synthesis as 28 strands of synthetic DNA described below. The strands were 14 pairs of single-strand DNAs having sequences that were partially complementary to each other. Each pair was annealed. Then, a double strand was prepared therefrom by an elongation reaction using DNA polymerase.

The synthetic DNA sequences of the 1st pair are as follows.

native_signal_1F:

(SEQ ID NO: 25)

CATGAAGACCTTGATCTTGGCTGTCGCTTTGGTCTACTGTGCTACTGTTC
ACTGTCAAGACTGTCCATAC
CLuc_mature_1R:

(SEQ ID NO: 26)

GATACATTCACCTTCTTTAGCTTCACAAGAGGTAGGGACAGTGTTCGGTG
GGTCTGGTTCGTATGGACAG

native_signal_IF is synthetic DNA comprising the initiation codon of mCLuc DNA and having a length of 69 bp from the codon in the 3′ region. CLuc_mature_IR is synthetic DNA having a strand complementary to a 70-bp sequence ranging from 60 bp to 129 bp of mCLuc DNA. The two synthetic DNAs each comprise a 10-bp complementary sequence. The DNA elongation reaction with the use of the two synthetic DNAs was carried out using 50 μl of a reaction solution containing 3 μM each of the synthetic DNAs, 200 μM of dNTP (a mixed solution of 4 types of deoxynucleotide triphosphates), 100 μM of MgSO₄, KOD Plus buffer (1×), and DNA polymerase (1 U) by the following steps: a first step at 94° C. for 2 minutes; a second step at 94° C. for 15 seconds (denaturation), 42° C. for 30 seconds (annealing), and 68° C. for 30 seconds (elongation) for 35 cycles; and a third step at 68° C. for 1 minute.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 1.”

Synthetic DNA sequences of the 2nd pair are as follows.

CLuc_mature_2F:

(SEQ ID NO: 27)

TGAATGTATCGATTCTTCTTGTGGTACTTGTACCAGGGACATATTGTCTG
ACGGTTTGTGTGAAAACAAG
CLuc_mature_3R:

(SEQ ID NO: 28)

TGCCGCTTCAACTCTGCATTCGATAACGTATTGGCACATTCTACAACAAG
TCTTGCCAGGCTTGTTTTCA

CLuc_mature_{—}2F is synthetic DNA having a length of 70 bp ranging from 120 bp to 189 bp of mCLuc DNA. CLuc_mature_{—}3R is synthetic DNA having a strand complementary to a 70-bp sequence ranging from 180 bp to 249 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 2.”

Synthetic DNA sequences of the 3rd pair are as follows:

CLuc_mature_4F:

(SEQ ID NO: 29)

TGAAGCGGCAGGGTGGTTCAGGACTTTTTACGGTAAACGTTTCCAATTCC
AAGAACCAGGTACTTACGTC;
and
CLuc_mature_5R:

(SEQ ID NO: 30)

ATCAAGATTCTCTAATGTTATGGATACTTTCCAATCTCCACCTTTGGTAC
CTTGGCCTAAGACGTAAGTA.

CLuc_mature_{—}4F is synthetic DNA having the length of 70 bp ranging from 240 bp to 309 bp of mCLuc DNA. CLuc_mature_{—}5R is synthetic DNA having a strand complementary to a 70-bp sequence ranging from 300 bp to 369 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 3.”

Synthetic DNA sequences of the 4th pair are as follows.

CLuc_mature_6F:

(SEQ ID NO: 31)

GAATCTTGATGGCACTAAGGGTGCTGTCTTAACTAAAACCCGGTTAGAAG
TCGCTGGTGATATTATCGAC
CLuc_mature_7R:

(SEQ ID NO: 32)

GGCGATGATAGGATCAGCACCACCATTAACAGTAATCGGATTCTCAGTAG
CTTGAGCGATGTCGATAATA.

CLuc_mature_{—}6F is synthetic DNA having a length of 70 bp ranging from 360 bp to 429 bp of mCLuc DNA. CLuc_mature_{—}7R is synthetic DNA having a strand complementary to a 70-bp sequence ranging from 420 bp to 489 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 4.”

Synthetic DNA sequences of the 5th pair are as follows.

CLuc_mature_8F:

(SEQ ID NO: 33)

TATCATCGCCAACCCTTATACTATCGGTGAGGTTACCATCGCCGTCGTTG
AGATGCCAGGCTTTAACATT
CLuc_mature_9R:

(SEQ ID NO: 34)

TCTGACAGAACGACCACCCAAGATGTCGATGACGATCAACTTGAAGAACT
CGATTACGGTAATGTTAAAG

CLuc_mature_{—}8F is synthetic DNA having a length of 70 bp ranging from 480 bp to 549 bp of mCLuc DNA. CLuc_mature_{—}9R is synthetic DNA having a strand complementary to a 70-bp sequence ranging from 540 bp to 609 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 5.”

Synthetic DNA sequences of the 6th pair are as follows.

CLuc_mature_10F:

(SEQ ID NO: 35)

TTCTGTCAGAATCGCTCCAGACACTGCTAACAAGGGTATGATCTCTGGTT
TGTGTGGCGATCTCAAGATG
CLuc_mature_11R:

(SEQ ID NO: 36)

GTTAATCTTAGGTTGGATAGCTAGCTGCTCAGGATCTGAAGTGAAATCAG
TGTCCTCCATCATCTTGAGA

CLuc_mature_{—}1 OF is synthetic DNA having a length of 70 bp ranging from 600 bp to 669 bp of mCLuc DNA. CLuc_mature_{—}11R is synthetic DNA having a strand complementary to a 70-bp sequence ranging from 720 bp to 789 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 6.”

Synthetic DNA sequences of the 7th pair are as follows.

CLuc_mature_12F:

(SEQ ID NO: 37)

TAAGATTAACCAAGAGTTCGATGGGTGTCCGTTATACGGTAATCCTGATG
ACGTCGCTTACTGTAAAGGC
CLuc_mature_13R:

(SEQ ID NO: 38)

TATGGTATAGTAGTAAAAGTTGATAGGATTCCTACAAGAATCTTTGTATG
GTTCCAACAAGCCTTTACAG.

CLuc_mature_{—}12F is synthetic DNA having a length of 70 bp ranging from 720 bp to 789 bp of mCLuc DNA. CLuc_mature_{—}13R is synthetic DNA having a strand complementary to a 70-bp sequence ranging from 780 bp to 849 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 7.”

Synthetic DNA sequences of the 8th pair are as follows.

CLuc_mature_14F:

(SEQ ID NO: 39)

CTATACCATATCTTGTGCTTTCGCCAGGTGCATGGGGGGAGACGAGCGTG
CATCTCATGTTTTGTTGGAC
CLuc_mature_15R:

(SEQ ID NO: 40)

AGTGTGACCAGACAAGACACAAGTACCTCTAGTTTCTGGAGCAGCACAAG
TCTCTCTGTAGTCCAACAAA

CLuc_mature_{—}14F is synthetic DNA having a length of 70 bp ranging from 840 bp to 909 bp of mCLuc DNA. CLuc_mature_{—}15R is synthetic DNA having a strand complementary to a 70-bp sequence ranging from 900 bp to 969 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 8.”

Synthetic DNA sequences of the 9th pair are as follows.

CLuc_mature_16F:

(SEQ ID NO: 41)

TGGTCACACTTTCTACGACACTTTCGACAAGGCTAGGTACCAATTCCAAG
GGCCGTGCAAGGAAATACTA
CLuc_mature_17R:

(SEQ ID NO: 42)

ATCCACGTTACGGTGAGAGACCTTGACGTCCCAAGTGTTCCAGAAGCAGT
CCGCAGCCATTAGTATTTCC

CLuc_mature_{—}16F is synthetic DNA having a length of 70 bp ranging from 960 bp to 1029 bp of mCLuc DNA. CLuc_mature_{—}17R is synthetic DNA having a strand complementary to a 70-bp sequence ranging from 1020 bp to 1089 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 9.”

Synthetic DNA sequences of the 10th pair are as follows.

CLuc_mature_18F:

(SEQ ID NO: 43)

TAACGTGGATTCTTACACTGAAGTCGAAAAGGTCAGGATTCGTAAGCAGT
CCACTGTCGTCGAATTGATC
CLuc_mature_19R:

(SEQ ID NO: 44)

TTGAGAGGAATATGGCACAGAAACGGCTTCACCACCGACCAAGATTTGCT
TACCGTCAACGATCAATTCG

CLuc_mature_{—}18F is synthetic DNA having a length of 70 bp ranging from 1080 bp to 1149 bp of mCLuc DNA. CLuc_mature_{—}19R is synthetic DNA having a strand complementary to a 70-bp sequence ranging from 1140 bp to 1209 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 10.”

Synthetic DNA sequences of the 11th pair are as follows.

CLuc_mature_20F:

(SEQ ID NO: 45)

TTCCTCTCAAAACACTTCAATATACTGGCAAGACGGTGACATCTTAACCA
CTGCTATCTTGCCAGAAGCC
CLuc_mature_21R:

(SEQ ID NO: 46)

GTCGAATGGGTCTCTAATATGAACAACTAATAGCTGCTTGAAGTTGAACT
TGACGACCAAGGCTTCTGGC

CLuc_mature_{—}20F is synthetic DNA having a length of 70 bp ranging from 1200 bp to 1269 bp of mCLuc DNA. CLuc_mature_{—}21R is synthetic DNA having a strand complementary to a 70-bp sequence ranging from 1260 bp to 1329 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 11.”

Synthetic DNA sequences of the 12th pair are as follows.

CLuc_mature_22F:

(SEQ ID NO: 47)

CCCATTCGACGGTAAGACTTGTGGTATCTGTGGTAACTACAATCAGGACT
TCTCTGACGATTCTTTCGAC
CLuc_mature_23R:

(SEQ ID NO: 48)

TGGCTTTTGTTCTTCAGTACAACCAGGTGGATTCGGTGTTAGATCGCAAG
CACCTTCAGCGTCGAAAGAA

CLuc_mature_{—}22F is synthetic DNA having a length of 70 bp ranging from 1320 bp to 1389 bp of mCLuc DNA. CLuc_mature_{—}23R is synthetic DNA having a strand complementary to a 70-bp sequence ranging from 1380 bp to 1449 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 12.”

Synthetic DNA sequences of the 13th pair are as follows.

CLuc_mature_24F:

(SEQ ID NO: 49)

ACAAAAGCCAGAAGCTGAACGTTTGTGCAACAGTTTATTCGCTGGTCAAT
CCGATTTGGACCAAAAGTGT
CLuc_mature_25R:

(SEQ ID NO: 50)

CCAAAAGTGTAACGTCTGTCACAAGCCAGACAGAGTTGAAAGATGTATGT
ACGAATACTGTTTGAGAGGT

CLuc_mature_{—}24F is synthetic DNA having a length of 70 bp ranging from 1440 bp to 1509 bp of mCLuc DNA. CLuc_mature_{—}25R is synthetic DNA having a strand complementary to a 70-bp sequence ranging from 1500 bp to 1569 bp of mCLuc DNA. The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 130 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 13.”

Synthetic DNA sequences of the 14th pair are as follows.

CLuc_mature_26F:

(SEQ ID NO: 51)

TTTGAGAGGTCAACAAGGTTTCTGTGACCATGCTTGGGAGTTCAAGAAGG
AGTGCTATATCAAGCACGGC
CLuc_mature_27R:

(SEQ ID NO: 52)

CCTACTTGCACTCATCTGGGACCTCTAAGGTATCGCCGTGCTTG

The above CLuc_mature_{—}26F is synthetic DNA having a length of 70 bp ranging from 1560 bp to 1629 bp of mCLuc DNA. CLuc_mature_{—}27R is synthetic DNA having a strand complementary to a 44-bp sequence ranging from 1620 bp to 1663 bp of mCLuc DNA, and including the termination codon (TAG). The two synthetic DNAs each comprise a 10-bp complementary sequence. The DNA elongation reaction with the use of the two synthetic DNAs was carried out under the same conditions used for producing the above DNA fragment 1.

The obtained PCR product was analyzed by 2% agarose electrophoresis such that a DNA fragment (of approximately 100 bp) was confirmed.

The obtained PCR product was purified using a GenElute PCR clean-up kit. Then, DNA was eluted from the column of the kit using 40 μl of distilled water. The obtained eluate was diluted 100-fold with distilled water. The resultant is referred to as “DNA fragment 14.”

(2) Linkage of mCLuc DNA Fragments

DNA fragments 1 to 14 obtained in (1) above were subjected to linkage by overlap PCR.

2-1. Linkage Between DNA Fragment 1 and DNA Fragment 2

DNA fragment 1 and DNA fragment 2 were linked to each other by PCR using the above native_signal_{—}1F (SEQ ID NO: 25) and CLuc_mature_{—}3R (SEQ ID NO: 28) as primers.

PCR was carried out using 50 μl of a reaction solution containing 300 nM each of the primers, 200 μM of dNTP (a mixed solution of 4 types of deoxynucleotide triphosphates), 100 μM of MgSO₄, 1 μl each of DNA fragments 1 and 2 as templates, KOD Plus buffer (1×), and DNA polymerase (1 U) by the following steps: a first step at 94° C. for 2 minutes; a second step at 94° C. for 15 seconds (denaturation), 42° C. for 30 seconds (annealing), and 68° C. for 30 seconds (elongation) for 35 cycles; and a third step at 68° C. for 1 minute.

The obtained PCR product was analyzed by 2% agarose electrophoresis such that a DNA fragment (of approximately 250 bp) was confirmed. Then, the total amount of the PCR product obtained was subjected to 2% agarose gel electrophoresis. A band (of approximately 250 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 15.”

2-2. Linkage Between DNA Fragment 3 and DNA Fragment 4

DNA fragment 3 and DNA fragment 4 were linked to each other by PCR using the above CLuc_mature_{—}4F (SEQ ID NO: 29) and CLuc_mature_{—}7R (SEQ ID NO: 32) as primers.

PCR was carried out using the above primers under the same conditions used for PCR in 2-1 above except that 1 μl each of DNA fragment 3 and DNA fragment 4 was used as a template.

The obtained PCR product was analyzed by 2% agarose electrophoresis such that a DNA fragment (of approximately 250 bp) was confirmed. Then, the total amount of the PCR product obtained was subjected to 2% agarose gel electrophoresis. A band (of approximately 250 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 16.”

2-3. Linkage Between DNA Fragment 5 and DNA Fragment 6

DNA fragment 5 and DNA fragment 6 were linked to each other by PCR using the above CLuc_mature_{—}8F (SEQ ID NO: 33) and CLuc_mature_{—}11R (SEQ ID NO: 36) as primers.

PCR was carried out using the above primers under the same conditions used for PCR in 2-1 above except that 1 μl each of DNA fragment 5 and DNA fragment 6 was used as a template.

The obtained PCR product was analyzed by 2% agarose electrophoresis such that a DNA fragment (of approximately 250 bp) was confirmed. Then, the total amount of the PCR product obtained was subjected to 2% agarose gel electrophoresis. A band (of approximately 250 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 17.”

2-4. Linkage Between DNA Fragment 7 and DNA Fragment 8

DNA fragment 7 and DNA fragment 8 were linked to each other by PCR using the above CLuc_mature_{—}12F (SEQ ID NO: 37) and CLuc_mature_{—}15R (SEQ ID NO: 40) as primers.

PCR was carried out using the above primers under the same conditions used for PCR in 2-1 above except that 1 μl each of DNA fragment 7 and DNA fragment 8 was used as a template.

The obtained PCR product was analyzed by 2% agarose electrophoresis such that a DNA fragment (of approximately 250 bp) was confirmed. Then, the total amount of the PCR product obtained was subjected to 2% agarose gel electrophoresis. A band (of approximately 250 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 18.”

2-5. Linkage Between DNA Fragment 9 and DNA Fragment 10

DNA fragment 9 and DNA fragment 10 were linked to each other by PCR using the above CLuc_mature_{—}16F (SEQ ID NO: 41) and CLuc_mature_{—}19R (SEQ ID NO: 44) as primers.

PCR was carried out using the above primers under the same conditions used for PCR in 2-1 above except that 1 μl each of DNA fragment 9 and DNA fragment 10 was used as a template.

The obtained PCR product was analyzed by 2% agarose electrophoresis such that a DNA fragment (of approximately 250 bp) was confirmed. Then, the total amount of the PCR product obtained was subjected to 2% agarose gel electrophoresis. A band (of approximately 250 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 19.”

2-6. Linkage Between DNA Fragment 11 and DNA Fragment 12

DNA fragment 11 and DNA fragment 12 were linked to each other by PCR using the above CLuc_mature_{—}20F (SEQ ID NO: 45) and CLuc_mature_{—}23R (SEQ ID NO: 48) as primers.

PCR was carried out using the above primers under the same conditions used for PCR in 2-1 above except that 1 μl each of DNA fragment 11 and DNA fragment 12 was used as a template.

The obtained PCR product was analyzed by 2% agarose electrophoresis such that a DNA fragment (of approximately 250 bp) was confirmed. Then, the total amount of the PCR product obtained was subjected to 2% agarose gel electrophoresis. A band (of approximately 250 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 20.”

2-7. Linkage Between DNA Fragment 13 and DNA Fragment 14

DNA fragment 13 and DNA fragment 14 were linked to each other by PCR using the above CLuc_mature_{—}24F (SEQ ID NO: 49) and CLuc_mature_{—}27R (SEQ ID NO: 52) as primers.

PCR was carried out using the above primers under the same conditions used for PCR in 2-1 above except that 1 μl each of DNA fragment 13 and DNA fragment 14 was used as a template.

The obtained PCR product was analyzed by 2% agarose electrophoresis such that a DNA fragment (of approximately 230 bp) was confirmed. Then, the total amount of the PCR product obtained was subjected to 2% agarose gel electrophoresis. A band (of approximately 230 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 21.”

2-8. Linkage Between DNA Fragment 16 and DNA Fragment 17

DNA fragment 16 and DNA fragment 17 were linked to each other by PCR using the above CLuc_mature_{—}4F (SEQ ID NO: 29) and CLuc_mature_{—}11R (SEQ ID NO: 36) as primers.

PCR was carried out using the above primers under the same conditions used for PCR in 2-1 above except that 1 μl each of DNA fragment 16 and DNA fragment 17 was used as a template.

The obtained PCR product was analyzed by 2% agarose electrophoresis such that a DNA fragment Cof approximately 490 bp) was confirmed. Then, the total amount of the PCR product obtained was subjected to 2% agarose gel electrophoresis. A band (of approximately 490 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 22.”

2-9. Linkage Between DNA Fragment 18 and DNA Fragment 19

DNA fragment 18 and DNA fragment 19 were linked to each other, by PCR using the above CLuc_mature_{—}12F (SEQ ID NO: 37) and CLuc_mature_{—}19R (SEQ ID NO: 44) as primers.

PCR was carried out using the above primers under the same conditions used for PCR in 2-1 above except that 1 μl each of DNA fragment 18 and DNA fragment 19 was used as a template.

The obtained PCR product was analyzed by 2% agarose electrophoresis such that a DNA fragment (of approximately 490 bp) was confirmed. Then, the total amount of the PCR product obtained was subjected to 2% agarose gel electrophoresis. A band (of approximately 490 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 23.”

2-10. Linkage Between DNA Fragment 20 and DNA Fragment 21

DNA fragment 20 and DNA fragment 21 were linked to each other by PCR using the above CLuc_mature_{—}20F (SEQ ID NO: 45) and CLuc_mature_{—}27R (SEQ ID NO: 52) as primers.

PCR was carried out using the above primers under the same conditions used for PCR in 2-1 above except that 1 μl each of DNA fragment 20 and DNA fragment 21 was used as a template.

The obtained PCR product was analyzed by 2% agarose electrophoresis such that a DNA fragment (of approximately 470 bp) was confirmed. Then, the total amount of the PCR product obtained was subjected to 2% agarose gel electrophoresis. A band (of approximately 470 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 24.”

2-11. Linkage Between DNA Fragment 15 and DNA Fragment 22

DNA fragment 15 and DNA fragment 22 were linked to each other by PCR using the above native_signal_{—}1F (SEQ ID NO: 25) and CLuc_mature_{—}11R (SEQ ID NO: 36) as primers.

PCR was carried out using 50 μl of a reaction solution containing 300 nM each of the primers, 200 μM of dNTP (a mixed solution of 4 types of deoxynucleotide triphosphates), 100 μM of MgSO₄, 1 μl each of DNA fragments 15 and 22 as templates, KOD Plus buffer (133 ), and DNA polymerase (1 U) by the following steps: a first step at 94° C. for 2 minutes; a second step at 94° C. for 15 seconds (denaturation), 42° C. for 30 seconds (annealing), and 68° C. for 1 minute (elongation) for 35 cycles; and a third step at 68° C. for 2 minutes.

The obtained PCR product was analyzed by 1% agarose electrophoresis such that a DNA fragment (of approximately 730 bp) was confirmed. Then, the total amount of the PCR product obtained was subjected to 1% agarose gel electrophoresis. A band (of approximately 730 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 25.”

2-12. Linkage Between DNA Fragment 23 and DNA Fragment 24

DNA fragment 23 and DNA fragment 24 were linked to each other by PCR using the above CLuc_mature_{—}12F (SEQ ID NO: 37) and CLuc_mature_{—}27R (SEQ ID NO: 52) as primers.

PCR was carried out using the above primers under the same conditions used for PCR in 2-11 above except that 1 μl each of DNA fragment 23 and DNA fragment 24 was used as a template.

The obtained PCR product was analyzed by 1% agarose electrophoresis such that a DNA fragment (of approximately 950 bp) was confirmed. Then, the total amount of the PCR product obtained was subjected to 1% agarose gel electrophoresis. A band (of approximately 950 bp) was cleaved out therefrom, followed by extraction of a DNA fragment by a gel extraction method using phenol/chloroform. The obtained DNA fragment was dissolved in 20 μl of distilled water, followed by 100-fold dilution with distilled water. The resultant is referred to as “DNA fragment 26.”

2-13. Linkage Between DNA Fragment 25 and DNA Fragment 26

DNA fragment 25 and DNA fragment 26 were linked to each other by PCR using primers described below.

nativeCLuc_5′SmaI:

(SEQ ID NO: 53)

CATACCCGGGATGAAGACCTTGATTTTGGC
mCLuc_3′XbaI:

(SEQ ID NO: 54)

CCCTGTCTAGACTACTTGCACTCATCTG

nativeCLuc_{—}5′SmaI has a 20-bp sequence in the 3′ region of mCLuc DNA comprising the initiation codon (ATG), such sequence having a SmaI site added to the 5′ region thereof. mCLuc_{—}3′XbaI is a sequence complementary to a 19-bp sequence in the 5′ upstream region of mCLuc DNA comprising the termination codon (TGA), such sequence having an XbaI site added to the 5′ region thereof.

PCR was carried out using 50 μl of a reaction solution containing 300 nM each of the primers, 200 μM of dNTP (a mixed solution of 4 types of deoxynucleotide triphosphates), 100 μM of MgSO₄, 1 μl each of DNA fragments 25 and 26 as templates, KOD Plus buffer (133 ), and DNA polymerase (1 U) by the following steps: a first step at 94° C. for 2 minutes; a second step at 94° C. for 15 seconds (denaturation), 42° C. for 30 seconds (annealing), and 68° C. for 2 minutes (elongation) for 35 cycles; and a third step at 68° C. for 4 minutes.

The obtained PCR product was analyzed by 1% agarose electrophoresis such that a DNA fragment (of approximately 1600 bp) was confirmed. Then, the total amount of the PCR product obtained was subjected to 1% agarose gel electrophoresis. A band (of approximately 1600 bp) was cleaved out therefrom, followed by extraction of a DNA fragment by a gel extraction method using phenol/chloroform. The obtained DNA fragment was dissolved in 20 μl of distilled water. The resultant is referred to as “DNA fragment 27.”

(3) Production of a Plasmid Comprising mCLuc DNA

3-1. Incorporation of DNA Fragment 27 Into the pZErO-2 Plasmid

In accordance with the manufacturer's protocols, 1 μg of the pZErO-2 plasmid (Invitrogen) was linearized with EcoRV, followed by phenol/chloroform extraction. The resultant was dissolved in 20 μl of distilled water so as to be used.

The above linearized pZErO-2 plasmid and DNA fragment 27 were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. Then, the extracted plasmid was subjected to analysis in terms of the restriction enzyme cleavage pattern and the nucleotide sequence. Thus, a transformant retaining a plasmid into which DNA fragment 27 had been inserted (hereafter to be referred to as “pZErO-2-mCLuc”) was identified.

3-2. Preparation of an mCLuc DNA Fragment

The Escherichia coli retaining pZErO-2-mCLuc obtained in 3-1 above was introduced into 50 ml of a liquid medium containing LB kanamycin (50 μg/ml), followed by shake culture at 37° C. for 16 hours. After the termination of culture, plasmid DNA was prepared in accordance with the manufacturer's protocols using a GenElute Plasmid midi prep kit. Then, absorbance at OD260 nm was measured such that DNA concentration was quantified.

A portion of the obtained DNA (5 μg) was cleaved with SmaI (50 U) in 50 μl of a reaction solution for 18 hours. After cleavage with SmaI, the resultant was purified using a GenElute PCR clean-up kit. Then, DNA was eluted from the column of the kit with 40 μl of distilled water. Thereafter, DNA was cleaved with XbaI (50 U) in 50 μl of a reaction solution for 18 hours, followed by purification using a GenElute PCR clean-up kit. Next, DNA was eluted from the column of the kit with 40 μl of distilled water. The resultant is referred to as “mCLuc DNA fragment.”

3-3. Preparation of pCLuRA+HindIII-SalI

Inverse PCR was carried out using primers described below and pCLuRA as a template in a manner such that HindIII and Sall sites were added to a position 50 bp away from the SpeI site in the 5′ region of pCLuRA, such site being located at the 5′ end of the MCS of pCLuRA.

Inverse_SalI_F:

(SEQ ID NO: 55)

CCCTGTCGACAAGCGCGCAATTAACCCTC
Inverse_HindIII-Sal_R:

(SEQ ID NO: 56)

CCCTGTCGACCCTAAGCTTGGCGTAATCATGGTCATAGC

Inverse_SalI_F has a nucleotide sequence corresponding to a 20-bp sequence 3′ downstream from a position 50 bp upstream in the 5′ region from the SpeI site, such nucleotide sequence having a Sall site added to the 5′ region thereof. In addition, Inverse_HindIII-Sal_R has a sequence complementary to a 21-bp nucleotide sequence 5′ upstream from a position 50 bp 5′ upstream from the SpeI site, such sequence having HindIII and SalI sites added to the 5′ region thereof.

PCR was carried out using 50 μl of a reaction solution containing 300 nM each of the primers, 200 μM of dNTP (a mixed solution of 4 types of deoxynucleotide triphosphates), 100 μM of MgSO₄, 5% DMSO, 10 ng of pCLuRA as a template, KOD Plus buffer (133 ), and DNA polymerase (1 U) by the following steps: a first step at 94° C. for 2 minutes; a second step at 94° C. for 15 seconds (denaturation), 52° C. for 30 seconds (annealing), and 68° C. for 8 minutes (elongation) for 35 cycles; and a third step at 68° C. for 10 minutes.

The obtained PCR product was analyzed by 1% agarose electrophoresis such that a DNA fragment (of approximately 7 kbp) was confirmed. The obtained PCR product was purified using a GenElute PCR clean-up kit. Then, DNA was eluted from the column of the kit with 40 μl of distilled water. The eluted DNA solution was subjected to cleavage with SalI (50 U) in 50 μl of a reaction solution for 18 hours. After cleavage with SalI, the resultant was purified using a GenElute PCR clean-up kit. Next, DNA was eluted from the column of the kit with 40 μl of distilled water.

A portion of the obtained eluate was subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. Then, the extracted plasmid was subjected to analysis in terms of the restriction enzyme cleavage pattern and the nucleotide sequence. Thus, a transformant retaining a plasmid to which a sequence of interest had been added was identified.

The obtained plasmid DNA was cleaved with HindIII (50 U) in 50 μl of a reaction solution for 18 hours. After cleavage with HindIII, the resultant was purified using a GenElute PCR clean-up kit. Then, DNA was eluted from the column of the kit with 40 μl of distilled water. Thereafter, DNA was cleaved with SalI (50 U) in 50 μl of a reaction solution for 18 hours, followed by purification using a GenElute PCR clean-up kit. Next, DNA was eluted from the column of the kit with 40 μl of distilled water. The resultant is referred to as a “pCLuRA+HindIII-SalI DNA fragment.”

3-4. Preparation of an SV40 poly(A)X2 DNA Fragment

In order to produce a DNA fragment in which two SV40 poly(A) signals were tandemly linked to each other (hereafter to be referred to as a “SV40 poly(A)X2 DNA fragment”), PCR was carried out using a pair of the 2 types of primers described below.

| | |
|---|---|
| polyA_HindIII_F: | |
| CCCTAAGCTTAGACATGATAAGATACATTG | (SEQ ID NO:57) |
| polyA_SacII_R: | |
| CCTACCGCGGTTACCACATTTGTAGAGG | (SEQ ID NO:58) |

polyA_HindIII_F is a primer having a 20-bp sequence in the 3′ region starting from the 5′ end of the SV40 poly(A) signal, such sequence having a HindIII site added to the 5′ region thereof. polyA_SacI_R is a primer having a sequence complementary to a 20-bp nucleotide sequence in the 5′ region starting from the 3′ end of the SV40 poly(A) signal, such sequence having a Sacll site added to the 5′ region thereof.

PCR was carried out using 50 μl of a reaction solution containing 300 nM each of the primers, 200 μM of dNTP (a mixed solution of 4 types of deoxynucleotide triphosphates), 100 μM of MgSO₄, 10 ng of SV40 genome DNA (Invitrogen) as a template, KOD Plus buffer (133 ), and DNA polymerase (1 U) by the following steps: a first step at 94° C. for 2 minutes; a second step at 94° C. for 15 seconds (denaturation), 52° C. for 30 seconds (annealing), and 68° C. for 1 minutes (elongation) for 35 cycles; and a third step at 68° C. for 2 minutes.

The obtained PCR product was analyzed by 1% agarose electrophoresis such that a DNA fragment (of approximately 500 bp) was confirmed. The obtained PCR product was purified using a GenElute PCR clean-up kit. Then, DNA was eluted from the column of the kit with 40 μl of distilled water. The eluted DNA solution was subjected to cleavage with SacIl (50 U) in 50 μl of a reaction solution for 18 hours. After cleavage with SacIl, the resultant was purified using a GenElute PCR clean-up kit. Next, DNA was eluted from the column of the kit with 40 μl of distilled water. The obtained DNA fragment is referred to as “SV40 poly(A)_{—}5′.”

In addition, PCR was carried out using the primers described below.

| | |
|---|---|
| polyA_SacII_F: | |
| CCTACCGCGGAGACATGATAAGATACATTG | (SEQ ID NO:59) |
| polyA_SalI_R: | |
| CCCTGTCGACTTACCACATTTGTAGAGG | (SEQ ID NO:60) |

polyA_SacII_F is a primer having a 20-bp sequence in the 3′ region starting from the 5′ end of the SV40 poly(A) signal, such sequence having a SacIh site added to the 5′ region thereof. polyA_SalI_R is a primer having a sequence complementary to a 20-bp nucleotide sequence in the 5′ region starting from the 3′ end of the SV40 poly(A) signal, such sequence having a SalI site added to the 5′ region thereof.

PCR was carried out using 50 μl of a reaction solution containing 300 nM each of the primers, 200 μM of dNTP (a mixed solution of 4 types of deoxynucleotide triphosphates), 100 μM of MgSO₄, 10 ng of SV40 genome DNA (Invitrogen) as a template, KOD Plus buffer (133 ), and DNA polymerase (1 U) by the following steps: a first step at 94° C. for 2 minutes; a second step at 94° C. for 15 seconds (denaturation), 52° C. for 30 seconds (annealing), and 68° C. for 1 minute (elongation) for 35 cycles; and a third step at 68° C. for 2 minutes.

The obtained PCR product was analyzed by 1% agarose electrophoresis such that a DNA fragment (of approximately 500 bp) was confirmed. The obtained PCR product was purified using a GenElute PCR clean-up kit. Then, DNA was eluted from the column of the kit with 40 μl of distilled water. The eluted DNA solution was subjected to cleavage with SacII (50 U) in 50 μl of a reaction solution for 18 hours. After cleavage with SacII, the resultant was purified using a GenElute PCR clean-up kit. Next, DNA was eluted from the column of the kit with 40 μl of distilled water. The obtained DNA fragment is referred to as “SV40 poly(A)_{—}3′.”

SV40 poly(A)_{—}5′ and SV40 poly(A)_{—}3′ obtained above were linked to each other using a DNA Ligation Kit. After the termination of ligation reaction, PCR was carried out using 1 μl of the reaction product as a template, a polyA_HindIII_F primer (SEQ ID NO: 57), and a polyA_SalI_R primer (SEQ ID NO: 60) under the same conditions used for production of the above SV40 poly(A)_{—}5′.

The obtained PCR product was analyzed by 1% agarose electrophoresis such that a band (of approximately 1 kbp) that was supposed to be a DNA fragment of interest was confirmed. Then, the entire PCR product obtained was subjected to 1% agarose gel electrophoresis. A band (of approximately 1 kbp) was cleaved out therefrom, followed by extraction of a DNA fragment by a gel extraction method using phenol/chloroform. The obtained DNA fragment was dissolved in 20 μl of distilled water.

The DNA fragment and 1 μl of the aforementioned linearized pZErO-2plasmid were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. Then, the extracted plasmid was subjected to analysis in terms of the restriction enzyme cleavage pattern and the nucleotide sequence. Thus, a transformant retaining a plasmid into which an SV40 poly(A)X2 DNA fragment had been inserted (hereafter to be referred to as “pZErO-2-SV40 poly(A)X2”) was identified.

pZErO-2-SV40 poly(A)X2 was cleaved with HindIII (50 U) in 50 μl of a reaction solution for 18 hours. After cleavage with HindIII, the resultant was purified using a GenElute PCR clean-up kit. Then, DNA was eluted from the column of the kit with 40 μl of distilled water. Thereafter, DNA was cleaved with SalI (50 U) in 50 μl of a reaction solution for 18 hours, followed by purification using a GenElute PCR clean-up kit. Next, DNA was eluted from the column of the kit with 40 μl of distilled water. The entire eluate was subjected to 1% agarose gel electrophoresis. A band (of approximately 1 kbp) was cleaved out therefrom, followed by extraction of a DNA fragment by a gel extraction method using phenol/chloroform. The obtained DNA fragment was dissolved in 20 μl of distilled water. The DNA fragment is referred to as an “SV40 poly(A)X2 DNA fragment.”

3-5. Production of pCLuRA+SV40 poly(A)X2

The pCLuRA+HindIII-SalI DNA fragment and the SV40 poly(A)X2 DNA fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. Then, the extracted plasmid was subjected to analysis in terms of the restriction enzyme cleavage pattern and the nucleotide sequence. Thus, a transformant retaining a plasmid into which DNA encoding a gene of interest had been inserted was identified. The plasmid DNA is hereafter referred to as “pCLuRA+SV40 poly(A)X2.”

3-6. Production of pUG35-MET25-EGFP3+SV40 poly(A)X2

pCLuRA+SV40 poly(A)X2 was cleaved with SmaI (50 U) in 50 μl of a reaction solution for 18 hours. After cleavage with SmaI, the resultant was purified using a GenElute PCR clean-up kit. Then, DNA was eluted from the column of the kit with 40 μl of distilled water. Thereafter, DNA was cleaved-with XbaI (50 U) in 50 μl of a reaction solution for 18 hours, followed by purification using a GenElute PCR clean-up kit. Next, DNA was eluted from the column of the kit with 40 μl of distilled water. The entire eluate was subjected to 1% agarose gel electrophoresis. A band (of approximately 6 kbp) was cleaved out therefrom, followed by extraction of a DNA fragment by a gel extraction method using phenol/chloroform. The obtained DNA fragment was dissolved in 20 μl of distilled water. The DNA fragment is referred to as “pUG35-MET25-EGFP3+SV40 poly(A)X2.”

3-7. Construction of pmCLuRA

The aforementioned mCLuc DNA fragment and pUG35-MET25-EGFP3+SV40 poly(A)X2 were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding a gene of interest had been inserted was identified. The plasmid into which the mCLuc DNA fragment had been inserted at a desired site is hereafter referred to as “pmCLuRA.”

### Example 11

### Comparison Between Reporter Assay Using mCLuc and Reporter Assay Using β-galactosidase

(1) Production of Plasmids (Reporter Plasmids) Obtained by Linking Each Promoter to the 5′ Upstream Region of mCLuc DNA

1-1. Cleavage of pmCLuRA

pmCLuRA was cleaved with SmaI (50 U) in 50 μl of a reaction solution for 18 hours. After cleavage with SmaI, the resultant was purified using a GenElute PCR clean-up kit. Then, DNA was eluted from the column of the kit with 40 μl of distilled water. Thereafter, DNA was cleaved with BamHI (50 U) in 50 μl of a reaction solution for 18 hours, followed by purification using a GenElute PCR clean-up kit. Next, DNA was eluted from the column of the kit with 40 μl of distilled water. The entire eluate was subjected to 1% agarose gel electrophoresis. A band (of approximately 7 kbp) was cleaved out therefrom, followed by extraction of a DNA fragment by a gel extraction method using phenol/chloroform. The obtained DNA fragment was dissolved in 20 μl of distilled water. The DNA fragment is referred to as the “pmCLuRA Bam HI-Sma I fragment.”

Likewise, pmCLuRA (5 μg) was cleaved with SmaI (50 U) in 50 μl of a reaction solution for 18 hours. After cleavage with SmaI, the resultant was purified using a GenElute PCR clean-up kit. Then, DNA was eluted from the column of the kit with 40 μl of distilled water. Thereafter, DNA was cleaved with SpeI (50 U) in 50 μl of a reaction solution for 18 hours, followed by purification using a GenElute PCR clean-up kit. Next, DNA was eluted from the column of the kit with 40 μl of distilled water. The entire eluate was subjected to 1% agarose gel electrophoresis. A band (of approximately 7 kbp) was cleaved out therefrom, followed by extraction of a DNA fragment by a gel extraction method using phenol/chloroform. The obtained DNA fragment was dissolved in 20 μl of distilled water. The DNA fragment is referred to as the “pmCLuRA Spe I-Sma I fragment.”

1-2. Isolation of the ACT1 Promoter and Incorporation of the Same into pmCLuRA

A promoter (SEQ ID NO: 61; hereafter to be referred to as “ACT1 promoter”) of the ACT1 gene (systematic gene name: YFL039C) of Saccharomyces cerevisiae was incorporated into pmCLuRA. Herein, the term “ACT1 promoter” indicates an untranslated region in the 5′ upstream region of the ACT1 gene; that is to say, a region sandwiched between the ACT1 gene and the YPT1 gene that is the 5′ upstream adjacent gene of the ACT1 gene (see the Yeast Genome Database: http://www.yeastgenome.org/).

PCR primer sequences (5′ACTI_Spe I and 3′ACT1) used for isolaiton of the ACT1 promoter were as follows.

| | |
|---|---|
| 5′ACT1_Spe I: | |
| CCCATTACTAGTACAAGCGCGCCTCTACC | (SEQ ID NO:62) |
| 3′ACT1: | |
| TGTTAATTCAGTAAATTTTCGATCTTG | (SEQ ID NO:63) |

The 5′ACT1_Spe I primer has a sequence in which a SpeI restriction enzyme site is added to the 5′ region of 17 bases 3′ downstream of the termination codon of YPT1 ORF 5′ upstream of ACT1 ORF. The 3′ACT1 primer has a sequence complementary to 27 bases 5′ upstream from the initiation codon of ACT1 ORF. In addition, see the Yeast Genome Database (http://www.yeastgenome.org/) regarding the positions of the primers.

PCR was carried out under basically the same conditions used for the above mature CLuc cDNA synthesis (Example 1) except that: Saccharomyces cerevisiae S288C genomic DNA (1 ng) was used as a template; 5′ACT1_Spe I (SEQ ID NO: 62) and 3′ACT1 (SEQ ID NO: 63) were used as primers; the annealing temperature was 48° C. and the elongations reaction time was 1 minute in a second step; and a third step was carried out for 2 minutes.

The obtained PCR product was analyzed by 1% agarose gel electrophoresis such that a DNA fragment (of approximately 670 bp) was confirmed. The obtained PCR product was purified using a GenElute PCR clean-up kit. Then, DNA was eluted from the column of the kit with 40 μl of distilled water. The obtained DNA solution was subjected to cleavage with SpeI (50 U) in 50 μl of a reaction solution for 18 hours, followed by purification using a GenElute PCR clean-up kit. Next, DNA was eluted from the column of the kit with 40 μl of distilled water. The obtained DNA fragment is referred to as the “ACT1 promoter DNA fragment.”

Subsequently, the ACT1 promoter DNA fragment and the pmCLuRA Spe I-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the ACT1 promoter had been inserted was identified. A plasmid (hereafter to be referred to as “pmCLuRA-ACT1”) was prepared from the transformant retaining a plasmid into which DNA encoding the ACT1 promoter had been inserted.

1-3. Isolation of the ADH1 Promoter and Incorporation of the Same into pmCLuRA

A promoter (SEQ ID NO: 64; hereafter to be referred to as “ADH1 promoter”) of the ADH1 gene (systematic gene name: YOL086C) of Saccharomyces cerevisiae was incorporated into pmCLuRA. Herein, the term “ADH1 promoter” indicates an untranslated region in the 5′ upstream region of the ADH1 gene; that is to say, a region sandwiched between the ADH1 gene and the YOL085C gene that is the 5′ upstream adjacent gene of the ADH1 gene (see the Yeast Genome Database: http://www.yeastgenome.org/).

PCR primer sequences (5′ADH1_BamH I and 3′ADH1) used for isolaiton of the ADH1 promoter were as follows.

| | |
|---|---|
| 5′ADH1_BamH I: | |
| CCCAGGATCCGTATACTAGAAGAATGAGCC | (SEQ ID NO:65) |
| 3′ADH1: | |
| TGTATATGAGATAGTTGATTGTATG | (SEQ ID NO:66) |

The 5′ADH1_Bam HI primer has a sequence in which a BamHI restriction enzyme site is added to the 5′ region of 20 bases 3′ downstream of the termination codon of YOL085C ORF 5′ upstream of ADH1 ORF. The 3′ADH1 primer has a sequence complementary to 25 bases 5′ upstream from the initiation codon of ADH1 ORF. In addition, see the Yeast Genome Database (http://www.yeastgenome.org/) regarding the positions of the primers.

PCR was carried out under basically the same conditions used for ACT1 promoter synthesis of 1-2 above except that 5′ADHl_Bam HI (SEQ ID NO: 65) and 3′ADH1 (SEQ ID NO: 66) were used as primers and the annealing temperature was 52° C. in a second step.

The obtained PCR product was analyzed by 1% agarose gel electrophoresis such that a DNA fragment (of approximately 1100 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The obtained DNA fragment is referred to as the “ADH1 promoter DNA fragment.”

Subsequently, the ADH1 promoter DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the ADH1 promoter had been inserted was identified. A plasmid (hereafter to be referred to as “pmCLuRA-ADH1”) was prepared from the transformant retaining a plasmid into which DNA encoding the ADH1 promoter had been inserted.

1-4. Isolation of the CYC1 Promoter and Incorporation of the Same into pmCLuRA

A promoter (SEQ ID NO: 67; hereafter to be referred to as “CYCL promoter”) of the CYC1 gene (systematic gene name: YJR048) of Saccharomyces cerevisiae was incorporated into pmCLuRA. Herein, the term “CYC1 promoter” indicates an untranslated region in the 5′ upstream region of the CYC1 gene; that is to say, a region sandwiched between the CYC1 gene and the ANB1 gene that is the 5′ upstream adjacent gene of the CYC1 gene (see the Yeast Genome Database: http://www.yeastgenome.org/).

PCR primer sequences (5′CYC1_BamH I and 3′CYC1) used for isolaiton of the CYC1 promoter were as follows.

| | |
|---|---|
| 5′CYC1_BamH I: | |
| CCCAGGATCCGTTTTAGTGTGTGAATGAAA | (SEQ ID NO:68) |
| 3′CYC1: | |
| TATTAATTTAGTGTGTGTATTTGTG | (SEQ ID NO:69) |

The 5′CYC1_Bam HI primer has a sequence in which a BamHI restriction enzyme site is added to the 5′ region of 20 bases 3′ downstream of the termination codon of ANBL ORF 5′ upstream of CYC1 ORF. The 3′CYC1 primer has a sequence complementary to 25 bases 5′ upstream from the initiation codon of CYC1 ORF. In addition, see the Yeast Genome Database (http://www.yeastgenome.org/) regarding the positions of the primers.

PCR was carried out under basically the same conditions used for ADH1 promoter synthesis of 1-3 above except that 5′CYC1_Bam HI (SEQ ID NO: 68) and 3′CYC1 (SEQ ID NO: 69) were used as primers.

The obtained PCR product was analyzed by 1% agarose gel electrophoresis such that a DNA fragment (of approximately 950 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The obtained DNA fragment is referred to as “CYC1 promoter DNA fragment.”

Subsequently, the CYC1 promoter DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the CYC1 promoter had been inserted was identified. A plasmid (hereafter to be referred to as “pmCLuRA-CYC1”) was prepared from the transformant retaining a plasmid into which DNA encoding the CYC1 promoter had been inserted.

1-5. Isolation of the TEF1 Promoter and Incorporation of the Same into pmCLuRA

A promoter (SEQ ID NO: 70; hereafter to be referred to as “TEF1 promoter”) of the TEF1 gene (systematic gene name: YPR080W) of Saccharomyces cerevisiae was incorporated into pmCLuRA. Herein, the term “TEF1 promoter” indicates an untranslated region in the 5′ upstream region of the TEF1 gene; that is to say, a region sandwiched between the TEF1 gene and the MRL1 gene that is the 5′ upstream adjacent gene of the TEF1 gene (see the Yeast Genome Database: http://www.yeastgenome.org/).

PCR primer sequences (5′TEF1_BamH I and 3′TEF1) used for isolation of the TEF1 promoter were as follows.

| | |
|---|---|
| 5′TEF1_BamH I: | |
| CCCAGGATCCACAATGCATACTTTGTACG | (SEQ ID NO:71) |
| 3′TEF1: | |
| TTTGTAATTAAAACTTAGATTAGATTGC | (SEQ ID NO:72) |

The 5′TEF1_Bam HI primer has a sequence in which a BamHI restriction enzyme site is added to the 5′ region of 19 bases 3′ downstream of the termination codon of MRL1 ORF 5′ upstream of TEF1 ORF. The 3′TEF1 primer has a sequence complementary to 28 bases 5′ upstream from the initiation codon of TEF1 ORF. In addition, see the Yeast Genome Database (http://www.yeastgenome.org/) regarding the positions of the primers.

PCR was carried out under basically the same conditions used for ADH1 promoter synthesis of 1-3 above except that 5′TEF1_Bam HI (SEQ ID NO: 71) and 3′TEF1 (SEQ ID NO: 72) were used as primers.

The obtained PCR product was analyzed by 1% agarose gel electrophoresis such that a DNA fragment (of approximately 580 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The obtained DNA fragment is referred to as “TEF1 promoter DNA fragment.”

Subsequently, the TEF1 promoter DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the TEF1 promoter had been inserted was identified. A plasmid (hereafter to be referred to as “pmCLuRA-TEF1”) was prepared from the transformant retaining a plasmid into which DNA encoding the TEF1 promoter had been inserted.

1-6. Isolation of the CUP1 Promoter and Incorporation of the Same Into pmCLuRA

A promoter (SEQ ID NO: 73; hereafter to be referred to as “CUP1 promoter”) of the CUP1 gene (systematic gene name: YHR053C) of Saccharomyces cerevisiae was incorporated into pmCLuRA. Herein, the term “CUP1 promoter” indicates an untranslated region in the 5′ upstream region of the CUP1 gene; that is to say, a region sandwiched between the CUP1 gene and the YHR054C gene that is the 5′ upstream adjacent gene of the CUP1 gene (see the Yeast Genome Database: http://www.yeastgenome.org/).

PCR primer sequences (5′CUP1_BamH I and 3′CUP1) used for isolaiton of the CUP1 promoter were as follows.

| | |
|---|---|
| 5′CUP1_BamH I: | |
| CCCAGGATCCTAAGCCGATCCCATTAC | (SEQ ID NO:74) |
| 3′CUP1: | |
| TTTATGTGATGATTGATTGATTGATTGTAC | (SEQ ID NO:75) |

The 5′CUPI_Bam HI primer has sequence in which a BamHI restriction enzyme site is added to the 5′ region of 17 bases 3′ downstream of the termination codon of YHR054C ORF 5′ upstream of CUP1 ORF. The 3′CUP1 primer has a sequence complementary to 30 bases 5′ upstream from the initiation codon of CUP1 ORF. In addition, see the Yeast Genome Database (http://www.yeastgenome.org/) regarding the positions of the primers.

PCR was carried out under basically the same conditions used for ADH1 promoter synthesis of 1-3 above except that 5′CUP1_Bam HI (SEQ ID NO: 74) and 3′CUP1 (SEQ ID NO: 75) were used as primers.

The obtained PCR product was analyzed by 1% agarose gel electrophoresis such that a DNA fragment (of approximately 460 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The obtained DNA fragment is referred to as “CUP1 promoter DNA fragment.”

Subsequently, the CUP1 promoter DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the CUP1 promoter had been inserted was identified. A plasmid (hereafter to be referred to as “pmCLuRA-CUP1”) was prepared from the transformant retaining a plasmid into which DNA encoding the CUP1 promoter had been inserted.

1-7. Isolation of the GAL1 Promoter and Incorporation of the Same Into pmCLuRA

A promoter (SEQ ID NO: 76; hereafter to be referred to as “GALL promoter”) of the GALl gene (systematic gene name: YBR020W) of Saccharomyces cerevisiae was incorporated into pmCLuRA. Herein, the term “GAL1 promoter” indicates an untranslated region in the 5′ upstream region of the GAL1 gene; that is to say, a region sandwiched between the GAL1 gene and the GAL10 gene that is the 5′ upstream adjacent gene of the GAL1 gene (see the Yeast Genome Database: http://www.yeastgenome.org/).

PCR primer sequences (5′GAL1_Spe I and 3′GAL1) used for isolation of the GAL1 promoter were as follows.

| | |
|---|---|
| 5′GAL1_Spe I: | |
| CCCAACTAGTACGGATTAGAAGCCGCCGAG | (SEQ ID NO:77) |
| 3′GAL1: | |
| GGTTTTTTCTCCTTGACGTTAAAG | (SEQ ID NO:78) |

The 5′GAL1_Spe I primer has a sequence in which a SpeI restriction enzyme site is added to the 5′ region of 20 bases 3′ downstream of the termination codon of GAL1 ORF 5′ upstream of GAL1 ORF. The 3′GAL1 primer has a sequence complementary to 24 bases 5′ upstream from the initiation codon of GAL1 ORF. In addition, see the Yeast Genome Database (http://www.yeastgenome.org/) regarding the positions of the primers.

PCR was carried out under basically the same conditions used for ADH1 promoter synthesis of 1-3 above except that 5′GAL1_Spe I (SEQ ID NO: 77) and 3′GAL1 (SEQ ID NO: 78) were used as primers.

The obtained PCR product was analyzed by 1% agarose electrophoresis such that a DNA fragment (of approximately 450 bp) was confirmed. The obtained PCR product was purified using a GenElute PCR clean-up kit. Then, DNA was eluted from the column of the kit with 40 μl of distilled water. The obtained DNA solution was subjected to cleavage with SpeI (50 U) in 50 μl of a reaction solution for 18 hours, followed by purification using a GenElute PCR clean-up kit. Next, DNA was eluted from the column of the kit with 40 μl of distilled water. The obtained DNA fragment is referred to as “GAL1 promoter DNA fragment.”

Subsequently, the GALL promoter DNA fragment and the pmCLuRA Spe I-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the GAL1 promoter had been inserted was identified. A plasmid (hereafter to be referred to as “pmCLuRA-GAL1”) was prepared from the transformant retaining a plasmid into which DNA encoding the GAL1 promoter had been inserted.

1-8. Isolation of the HSP12 Promoter and Incorporation of the Same Into pmCLuRA

A promoter (SEQ ID NO: 79; hereafter to be referred to as “HSP12 promoter”) of the HSP12 gene (systematic gene name: YFL014W) of Saccharomyces cerevisiae was incorporated into pmCLuRA. Herein, the term “HSP 12 promoter” indicates an untranslated region in the 5′ upstream region of the HSP12 gene; that is to say, a region sandwiched between the HSP12 gene and the YFL015W-A gene that is the 5′ upstream adjacent gene of the HSP12 gene (see the Yeast Genome Database: http://www.yeastgenome.org/).

PCR primer sequences (5′HSP12_BamH I and 3′HSP12) used for isolation of the HSP12 promoter were as follows.

| | |
|---|---|
| 5′HSP12_BamH I: | |
| GGGTGGATCCGATCCCACTAACGGCCCAG | (SEQ ID NO:80) |
| 3′HSP12: | |
| TGTTGTATTTAGTTTTTTTTGTTTTGAG | (SEQ ID NO:81) |

The 5′HSP12_Bam HI primer has a sequence in which a BamHI restriction enzyme site is added to the 5′ region of 17 bases 3′ downstream of the termination codon of YFL015W-A ORF 5′ upstream of HSP12 ORF. The 3′HSP12 primer has a sequence complementary to 30 bases 5′ upstream from the initiation codon of HSP12 ORF. In addition, see the Yeast Genome Database (http://www.yeastgenome.org/) regarding the positions of the primers.

PCR was carried out under basically the same conditions used for ADH1 promoter synthesis of 1-3 above except that 5′HSP12_Bam HI (SEQ ID NO: 80) and 3′HSP12 (SEQ ID NO: 81) were used as primers.

The obtained PCR product was analyzed by 1% agarose electrophoresis such that a DNA fragment (of approximately 610 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The obtained DNA fragment is referred to as “HSP12 promoter DNA fragment.”

Subsequently, the HSP12 promoter DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the HSP12 promoter had been inserted was identified. A plasmid (hereafter to be referred to as “pmCLuRA-HSP12”) was prepared from the transformant retaining a plasmid into which DNA encoding the HSP12 promoter had been inserted.

1-9. Incorporation of the TDH3 Promoter into pmCLuRA

The TDH3 promoter DNA fragment that had been isolated in Example 1 and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the TDH3 promoter had been inserted was identified. A plasmid (hereafter to be referred to as “pmCLuRA-TDH3”) was prepared from the transformant retaining a plasmid into which DNA encoding the TDH3 promoter had been inserted.

(2) Transformation of Yeast (Saccharomyces cerevisiae Strain YPH500) With the Use of Each Reporter Plasmid Comprising mCLuc DNA

Saccharomyces cerevisiae strain YPH500 was transformed with the use of the following 8 plasmids: pmCLuRA-ACT1, pmCLuRA-ADH1, pmCLuRA-CYC1, pmCLuRA-TDH3, pmCLuRA-TEF1, pmCLuRA-CUP1, pmCLuRA-GAL1, and pmCLuRA-HSP12. An EZ-transformation kit (BIO101) was used for such transformation.

The obtained transformant was applied to a uracil-free synthetic agar medium (SD+KHLadeW), followed by culture at 30° C. for 3 days.

After culture for 3 days, transformants each comprising a different plasmid were obtained.

(3) Measurement of the Activity of Each Promoter Based on the mCLuc

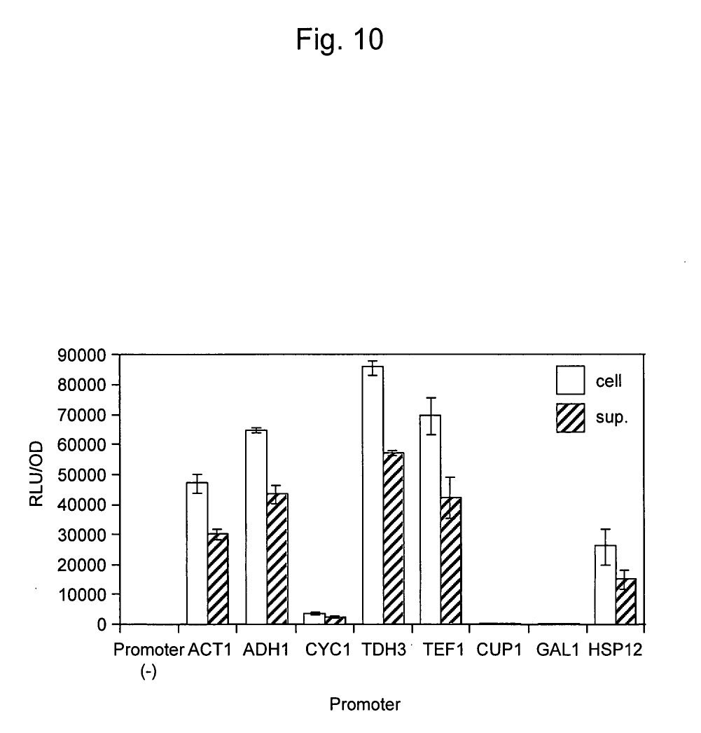

Three colonies each of a different single clone of a transformant comprising a plasmid obtained in (2) above were introduced into a synthetic liquid medium (SD+KHLadeW 200 mM KPi), followed by overnight culture. Then, 5 μl each of the obtained culture solutions that had reached the stationary phase after overnight culture was introduced into each well of a 96-deep well plate, with 1 ml of the same medium having been added to each such well. This was followed by shake culture at 30° C. 16 hours after the initiation of culture, a portion of each culture solution was recovered, followed by measurement of absorbance at 600 nm (OD600) and relative light units (RLU) from a luminometer in accordance with the method in Example 1. The activity of each promoter was digitized based on the obtained values. Specifically, mCLuc activity obtained by dividing relative light units by absorbance at 600 nm indicates the relative value of the transcriptional activity of each promoter. FIG. 10 shows the results. In FIG. 10, the expression “cell” indicates mCLuc activity measured using a culture solution containing yeast cells; and the expression “sup.” indicates mCLuc activity measured using a culture supernatant obtained by centrifugation of a culture solution at 15,000 rpm. Also, “Promoter (−)” indicates a result obtained from a transformant having a plasmid not comprising a promoter.

(4) Construction of the β-galactosidase Reporter Plasmid (pGALuRA)

In order to construct a reporter plasmid using β-galactosidase, cDNA of β-galactosidase was prepared by PCR using the primers described below.

| | |
|---|---|
| LacZ_5′SmaI_F: | |
| GGGTCCCGGGATGACCGGTTCCGGAGCTTG | (SEQ ID NO:82) |
| LacZ_3′XbaI_R: | |
| CCCTGTCTAGATTACGCGAAATACGGGCAG | (SEQ ID NO:83) |

The LacZ_{—}5′SmaI_F primer has a sequence in which a SmaI restriction enzyme site is added to the the 5′ region of 20 bases 3′ downstream of the initiation codon of β-galactosidase ORF. The LacZ_{—}3′XbaI_R primer has a sequence complementary to 19 bases 5′ upstream from the termination codon of β-galactosidase ORF.

PCR was carried out under basically the same conditions used for the above mature CLuc cDNA synthesis (Example 1) except that: 10 ng of pJM133 DNA (Genes Develop., 7, 833-843 (1993)) was used as a template; LacZ_{—}5′SmaI_F (SEQ ID NO: 82) and LacZ_{—}3′XbaI_R (SEQ ID NO: 83) were used as primers; the annealing temperature was 52° C. and the elongation reaction time was 4 minutes in a second step; and a third step was carried out for 8 minutes.

The obtained PCR product. was analyzed by 1% agarose gel electrophoresis such that a DNA fragment (of approximately 3.3 kbp) was confirmed. The obtained PCR product was purified using a GenElute PCR clean-up kit. Then, DNA was eluted from the column of the kit with 40 μl of distilled water. The obtained DNA solution was subjected to cleavage with SmaI (50 U) in 50 μl of a reaction solution for 18 hours, followed by purification using a GenElute PCR clean-up kit. Next, DNA was eluted from the column of the kit with 40 μl of distilled water. The obtained DNA solution was subjected to cleavage with XbaI (50 U) in 50 μl of a reaction solution for 18 hours, followed by purification using a GenElute PCR clean-up kit. Thereafter, DNA was eluted from the column of the kit with 40 μl of distilled water. The obtained DNA fragment is referred to as the “β-galactosidase DNA fragment.”

The β-galactosidase DNA fragment and 1 μl of pUG35-MET25-EGFP3+SV40 poly(A)X2 were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding β-galactosidase had been inserted was identified. A plasmid (hereafter to be referred to as “pGALuRA ”) was prepared from the transformant retaining a plasmid into which DNA encoding β-galactosidase had been inserted.

Subsequently, the Escherichia coli retaining pGALuRA was introduced into 50 ml of a liquid medium containing LB ampicillin (50 μg/ml), followed by shake culture at 37° C. for 16 hours. After the termination of culture, plasmid DNA was prepared in accordance with the manufacturer's protocols using a GenElute Plasmid midi prep kit. Then, absorbance at OD260 nm was measured so as to quantify DNA concentration.

A portion of the obtained DNA (5 μg) was cleaved with SmaI (50 U) in 50 μl of a reaction solution for 18 hours. After cleavage with SmaI, the resultant was purified using a GenElute PCR clean-up kit. Then, DNA was eluted from the column of the kit with 40 μl of distilled water. Thereafter, DNA was cleaved with BamHI (50 U) in 50 μl of a reaction solution for 18 hours, followed by purification using a GenElute PCR clean-up kit. Then, DNA was eluted from the column of the kit with 40 μl of distilled water. The entire eluate was subjected to 1% agarose gel electrophoresis. A band (of approximately 7 kbp) was cleaved out therefrom, followed by extraction of a DNA fragment by a gel extraction method using phenol/chloroform. The obtained DNA fragment was dissolved in 20 μl of distilled water. The DNA fragment is referred to as the “pGALuRA Bam HI-Sma I fragment.”

Likewise, pGALuRA (5 μg) was cleaved with SmaI (50 U) in 50 μl of a reaction solution for 18 hours. After cleavage with SmaI, the resultant was purified using a GenElute PCR clean-up kit. Then, DNA was eluted from the column of the kit with 40 μl of distilled water. Thereafter, DNA was cleaved with SpeI (50 U) in 50 μl of a reaction solution for 18 hours, followed by purification using a GenElute PCR clean-up kit. Next, DNA was eluted from the column of the kit with 40 μl of distilled water. The entire eluate was subjected to 1% agarose gel electrophoresis. A band (of approximately 7 kbp) was cleaved out therefrom, followed by extraction of a DNA fragment by a gel extraction method using phenol/chloroform. The obtained DNA fragment was dissolved in 20 μl of distilled water. The DNA fragment is referred to as the “pGALuRA Spe I-Sma I fragment.”

(5) Incorporation of Different Promoters into pGALuRA

As with the case of incorporation of different promoters into pmCLuRA in (1) above, the ACT1 promoter DNA fragment, the ADH1 promoter DNA fragment, the CYC1 promoter DNA fragment, the TDH3 promoter DNA fragment, the TEF1 promoter DNA fragment, the CUP1 promoter DNA fragment, the GAL1 promoter DNA fragment, and the HSP12 promoter DNA fragment were separately incorporated into pGALuRA. The obtained plasmids are referred to as “pGALuRA-ACT1,” “pGALuRA-ADH1,” “pGALuRA-CYC 1,” “pGALuRA-TDH3,” “pGALuRA-TEF 1,” “pGALuRA-CUP 1,” “pGALuRA-GAL1,” and “pGALuRA-HSP12,” respectively.

(6) Transformation of Yeast (Saccharomyces cerevisiae Strain YPH500) With the Use of Each Reporter Plasmid Comprising DNA Encoding β-galactosidase

Saccharomyces cerevisiae strain YPH500 was transformed with the use of the following 8 plasmids: pGALuRA-ACT1, pGALuRA-ADH1, pGALuRA-CYC1, pGALuRA-TDH3, pGALuRA-TEF1, pGALuRA-CUP1, pGALuRA-GAL1, and pGALuRA-HSP12. An EZ-transformation kit (BIO101) was used for transformation.

The obtained transformant was applied to an uracil-free synthetic agar medium (SD+KHLadeW), followed by culture at 30° C. for 3 days.

After culture for 3 days, transformants each comprising a different plasmid were obtained.

(7) Preparation of a Sample of a Cell Disruption Supernatant

Three colonies each of a different single clone of a transformant comprising a plasmid obtained in (6) above were introduced into a synthetic liquid medium (SD+KHLadeW 200 mM KPi), followed by overnight culture. Then, 100 μl each of the obtained culture solutions that had reached the stationary phase after overnight culture was added to 20 ml of the same medium, followed by shake culture at 30° C. 16 hours after the initiation of culture, a portion of each culture solution was recovered, followed by measurement of OD600. Then, cells at the logarithmic phase were recovered. The cells recovered were again agitated in 500 μl of CelLytic (Sigma) so as to be vortexed at 4° C. for approximately 1 hour in the presence of tungsten beads for cell disruption. After cell disruption, centrifugation was carried out so as to obtain the supernatant. The obtained supernatant (extract) was quantified in terms of protein concentration using a modified procedure of the method of Lowry et al. (J. Biol. Chem., 193, 265-275 (1951)).

(8) β-galactosidase Assay

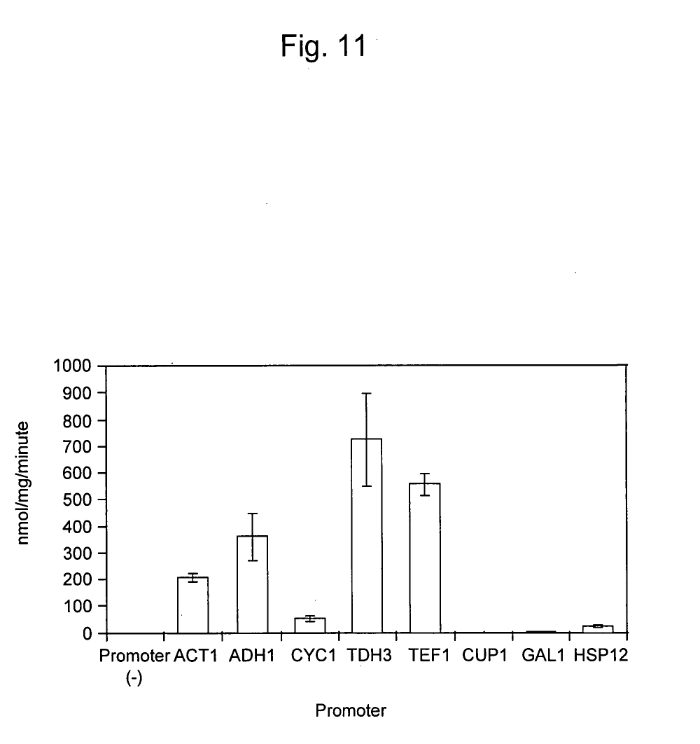

β-galactosidase assay was carried out by the method of Rose and Botein (1983, Methods Enzymol. 101: 167-180) using the extract obtained in (7) above. Z buffer was added to the extract and the volume of the obtained solution was adjusted using breaking buffer. The resulting solution was incubated at 28° C. for 5 minutes. Thereafter, an ONPG (o-nitrophenyl-β-D-galactopyranoside) solution was added thereto, thereby initiating reaction. The reaction solution was incubated at 28° C. until the color thereof became yellow. Thereafter, reaction was developed using a Na₂CO₃ solution, followed by measurement of absorbance at 420 nm. Based on the values obtained, promoter activity was calculated using the following calculating formula: OD420×1.7/(0.0045×protein amount×extract amount×time). That is, β-galactosidase activity corresponds to the relative value of the transcriptional activity of each promoter. FIG. 11 shows the results. In FIG. 11, the expression “Promoter (−)” indicates a result obtained from a transformant having a plasmid not comprising a promoter.

(9) Comparison Between Reporter Assay Using mCLuc and Reporter Assay Using β-galactosidase

As is apparent from FIGS. 10 and 11, there was a strong correlation between the relative activity of each promoter measured using mCLuc as a reporter enzyme and the relative activity of each promoter measured by the conventional method using β-galactosidase as a reporter enzyme. Based on these results, it has been found that results obtained by the method using mCLuc as a reporter enzyme were similar to those obtained by the conventional method using β-galactosidase as a reporter enzyme, meaning that the conventional method can be replaced by the method using mCLuc as a reporter enzyme. Further, in view of the necessary amount of culture solution and ease of measurement protocols, it has been found that the above method is more suitable for high-throughput assay than the conventional β-galactosidase method.

### Example 12

### Comparison Between the Results of Reporter Assay Using mCLuc and the Results of Measurement of Intracellular mRNA Content

mCLuc mRNA content was quantified so as to examine whether or not the results of assay using the reporter plasmid pmCLuRA in Example 11 reflected the intracellular expression level of mRNA.

(1) Preparation of Samples

Three colonies each of a different single clone of a transformant comprising a plasmid (pmCLuRA-ACT1, pmCLuRA-ADH1, pmCLuRA-CYCI, pmCLuRA-TDH3, pmCLuRA-TEF1, pmCLuRA-CUP1, pmCLuRA-GAL1, or pmCLuRA-HSP12) obtained in Example 11 were introduced into a synthetic liquid medium (SD+KHLadeW 200 mM KPi), followed by overnight culture. Then, 100 μl of the obtained culture solution that had reached the stationary phase after overnight culture was added to 20 ml of the same medium, followed by shake culture at 30° C. for approximately 16 hours. When the value of OD600 reached the mid-log phase, culture was terminated and the cells were recovered so as to be stored at −80° C. RNA was prepared using an RNeasy mini kit (QIAGEN). The obtained total RNA was subjected to reverse transcription using ReverTra Ace (TOYOBO) such that cDNA was synthesized.

(2) Production of a Control Plasmid

The TDH3 gene was selected as a control gene necessary for mRNA measurement by real-time PCR. Then, the ORF of the gene was cloned into pZErO-2 so as to obtain a control plasmid. PCR was carried out using the primers shown below so as to obtain the TDH3 gene.

(SEQ ID NO: 84)

TDH3_CDS_F: ATGGTTAGAGTTGCTATTAACGGTTTCG

(SEQ ID NO: 85)

TDH3_CDS_R: TTAAGCCTTGGCAACGTGTTCAACCAAG

The TDH3_CDS_F primer has a 28-bp sequence 3′ downstream from the initiation codon of TDH3 ORF. The TDH3_CDS_R primer has a sequence comprementary to a 28-bp sequence 5′ upstream from the termination codon of TDH3 ORF.

PCR was carried out under basically the same conditions used for the above mature CLuc cDNA synthesis (Example 1) except that: Saccharomyces cerevisiae S288C genomic DNA (1 ng) was used as a template; TDH3_CDS_F (SEQ ID NO: 84) and TDH3_CDS_R (SEQ ID NO: 85) were used as primers; the annealing temperature was 55° C. and the elongation reaction time was 1 minute in a second step; and a third step was carried out for 2 minutes.

The obtained PCR product was analyzed by 1% agarose gel electrophoresis such that a DNA fragment (of approximately 1 kbp) was confirmed. The obtained PCR product was purified using a GenElute PCR clean-up kit. Then, DNA was eluted from the column of the kit with 40 μl of distilled water.

The obtained DNA and the aforementioned linearized pZErO-2 were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding TDH3 ORF had been inserted was identified. The plasmid is referred to as “pZErO-2-TDH3 ORF.”

(3) Real-time PCR

The two primers used for real-time PCR described below were designed using the primer design software Primer Express (ABI).

(SEQ ID NO: 86)

mCLuc_RT_F: GGGCCGTGCAAGGAAAT

(SEQ ID NO: 87)

mCLuc_RT_R: GACGTCCCAAGTGTTCCAGAA

The mCLuc_RT_F primer has a nucleotide sequence from 1009 bp to 1025 bp 3′ downstream from the initiation codon of mCLuc ORF. The mCLuc_RT_R primer has a nucleotide sequence complementary to a sequence from 1045 bp to 1066 bp 3′ downstream from the initiation codon of mCLuc ORF.

Meanwhile, the two primers described below used for detection of the TDH3 gene used for quantification of a control plasmid were also designed using the primer design software Primer Express (ABI).

(SEQ ID NO: 88)

TDH3_RT_F: CTGCTAAGGCTGTCGGTAAGGT

(SEQ ID NO: 89)

TDH3_RT_R: TGAAAGCCATACCGGTCAACT

The TDH3_RT_F primer has a nucleotide sequence from 632 bp to 653 bp 3′ downstream from the initiation codon of TDH3 ORF. Meanwhile, the TDH3_RT_R primer has a nucleotide sequence complementary to a sequence from 674 bp to 694 bp 3′ downstream from the initiation codon of TDH3 ORF.

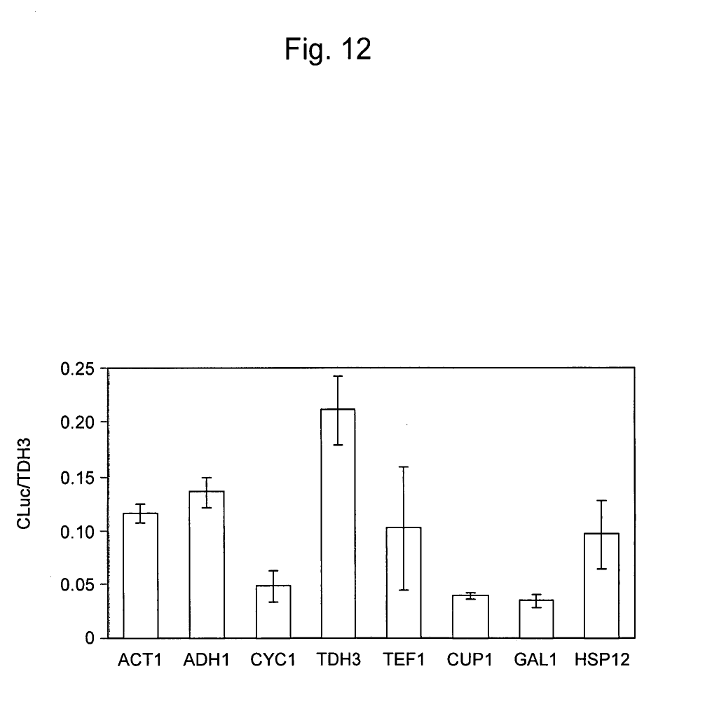

Real-time PCR was carried out using 20 μl of a reaction solution (10 μl of the master mix; 50 nM each of the primers; and 5 ng of cDNA) using a Power SYBR Green PCR Master Mix (ABI). PCR was carried out by the following steps: a first step at 95° C. for 10 minutes; and a second step at 95° C. for 15 seconds (denaturation) and at 60° C. for 60 seconds (annealing/elongation) for 40 cycles. FIG. 12 shows the results. The results shown in FIG. 12 were obtained by relative quantification of the mCLuc mRNA content with the use of the control plasmid as an internal standard.

As is apparent from FIGS. 10 and 12, there was a strong correlation between the results obtained using mCLuc as a reporter enzyme and the mCLuc mRNA contents measured by real-time PCR. Thus, reporter assay using mCLuc as a reporter enzyme was found to be an excellent measurement system used for transcriptional activity measurement.

### Example 13

### Measurement of Promoter Induction Caused By Chemical Substances Upon Reporter Assay Using mCLuc

(1) The CuSO₄ Induction Experiment

It has been known that the expression of the CUP1 gene (systematic gene name: YHR053C) of Saccharomyces cerevisiae is induced in the presence of copper ions (Gene, 48, 13-22 (1986)). That is, a CUP1 promoter is a copper-ion-inducible promoter. Thus, with the use of pmCLuRA-CUP1, it was examined whether or not such induction would be observed in the case of mCLuc. In addition, a similar experiment was carried out using β-galactosidase. The results of both experiments were compared and examined.

Three clones of a transformant comprising pmCLuRA-CUP1 and those of a transformant comprising pGALuRA-CUP1 described above were separately introduced into a synthetic liquid medium (SD+KHLadeW 200 mM KPi). Each resultant was shake-cultured overnight at 30° C. until the stationary phase.

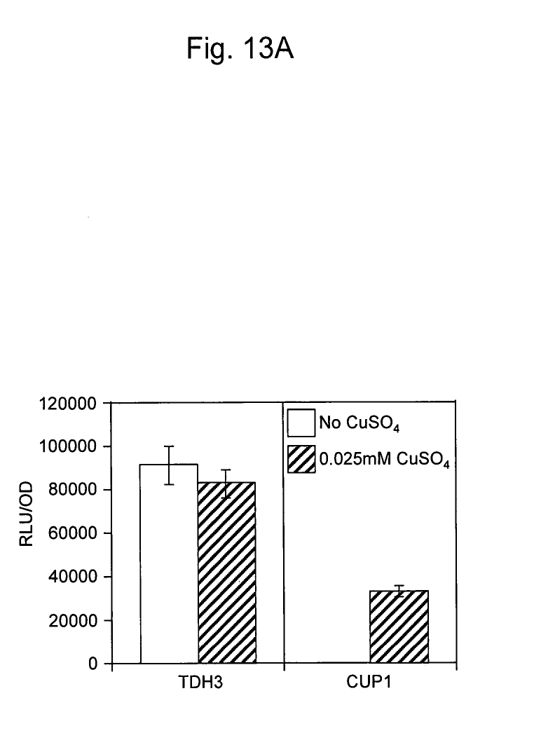

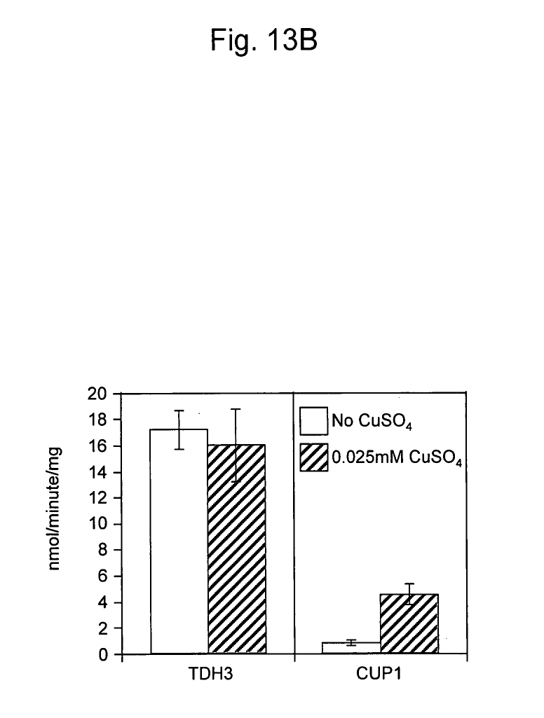

Subsequently, 5 μl of the culture solution containing a transformant comprising pmCLuRA-CUP1 was introduced into each well of a 96-deep well plate, with 1 ml of a 0.025 mM CuSO₄-containing synthetic liquid medium (SD+KHLadeW 200 mM KPi) or a CuSO₄-free synthetic liquid medium (SD+KHLadeW 200 mM KPi) having been added to such well. This was followed by shake culture at 30° C. 8 hours after the initiation of culture, a portion of the culture solution was recovered, followed by measurement of absorbance at 600 nm (OD600) and relative light units (RLU) from a luminometer in accordance with the method in Example 1. The activity of each promoter was digitized based on the obtained values, as with the case of Example 11. In addition, a transformant comprising pmCLuRA-TDH3 as a control was also subjected to a similar experiment. FIG. 13A shows the results.

Meanwhile, 100 μl of a culture solution containing a transformant comprising pGALuRA-CUP1 was introduced into 20 ml of a 0.025 mM CuSO₄-containing synthetic liquid medium (SD+LHLadeW 200 mM KPi) or a CuSO₄-free synthetic liquid medium (SD+LHLadeW 200 mM KPi), followed by shake culture at 30° C. 16 hours after the initiation of culture, a portion of the culture solution was recovered, followed by measurement of OD600. Then, cells at the logarithmic phase were recovered. Subsequently, the promoter activity was digitized by carrying out β-galactosidase assay as with the case of Example 11. In addition, a transformant comprising pGALuRA-TDH3 as a control was also subjected to a similar experiment. FIG. 13B shows the results.

(2) The Galactose Induction Experiment

It has been known that the expression of GAL1 gene (systematic gene name: YBR020W) of Saccharomyces cerevisiae is induced in the absence of glucose and the presence of galactose (West, R. W. J. et al., Mol. Cell. Biol., 4: 2467-2478 (1984)). That is, a GAL1 promoter is a galactose-inducible promoter. Thus, with the use of pmCLuRA-GAL1, it was examined whether or not such induction would be observed in the case of mCLuc. In addition, a similar experiment was carried out using β-galactosidase. The results of both experiments were compared and examined.

Three clones of a transformant comprising pmCLuRA-GAL1 and those of a transformant comprising pGALuRA-GAL1 described above were separately introduced into a synthetic liquid medium (SD+KHLadeW 200 mM KPi). Each resultant was shake-cultured overnight at 30° C. until the stationary phase.

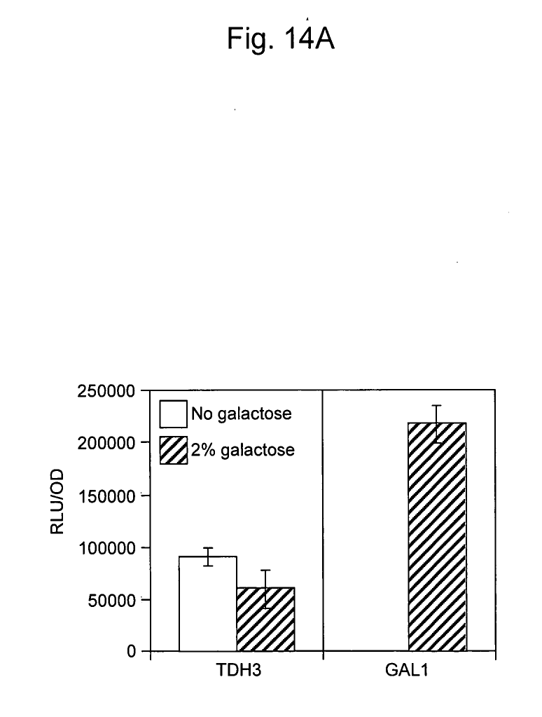

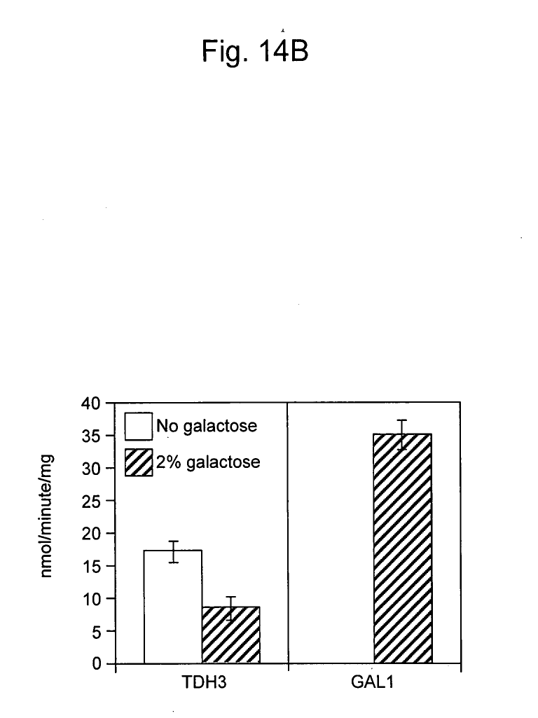

Subsequently, 20 μl of the culture solution containing a transformant comprising pmCLuRA-GAL1 was introduced into each well of a 96-deep well plate, with 1 ml of a 2% galactose-containing synthetic liquid medium (SC+KHLadeW (prepared by removing glucose from SD+KHLWade in Example 1) 200 mM KPi) or a synthetic liquid medium (SD+KHLadeW 200 mM KPi) having been added to such well. This was followed by shake culture at 30° C. 32 hours after the initiation of culture, a portion of the culture solution was recovered, followed by measurement of absorbance at 600 nm (OD600) and relative light units (RLU) from a luminometer in accordance with the method in Example 1. The activity of each promoter was digitized based on the obtained values as with the case of Example 11. In addition, a transformant comprising pmCLuRA-TDH3 as a control was also subjected to a similar experiment. FIG. 14A shows the results.

Meanwhile, 500 μl of a culture solution containing a transformant comprising pGALuRA-GAL1 was introduced into 20 ml of a 2% galactose-containing synthetic liquid medium (SC+KHLadeW 200 mM KPi) or a synthetic liquid medium (SD+KHLadeW 200 mM KPi), followed by shake culture at 30° C. 32 hours after the initiation of culture, a portion of the culture solution was recovered, followed by measurement of OD600. Thus, cells at the logarithmic phase were recovered. Subsequently, the promoter activity was digitized by carrying out β-galactosidase assay as with the case of Example 11. In addition, a transformant comprising pGALuRA-TDH3 as a control was also subjected to a similar experiment. FIG. 14B shows the results.

(3) Measurement Results of Promoter Induction Caused By Chemical Substances

As shown in FIGS. 13A, 13B, 14A, and 14B, regarding transcription induction of the CUP1 promoter caused by copper ions and transcription induction of the GAL1 promoter caused by galactose, there was a strong correlation between the method for measuring mCLuc as a reporter, enzyme and the conventional method using β-galactosidase as a reporter enzyme. Based on the result, it has been found that the results of promoter transcription induction caused by chemical substances obtained by the method using mCLuc as a reporter enzyme are similar to those obtained by the conventional method using β-galactosidase as a reporter enzyme, meaning that the conventional method can be replaced by the method using mCLuc. Further, in terms of the necessary amount of a culture solution and the ease of measurement protocols, it was found that the above method is more suitable for high-throughput assay than the conventional β-galactosidase method.

### Example 14

### Search for a Cis Sequence Involved in Transcriptional Activation Within a Promoter Upon Reporter Assay Using mCLuc

(1) Production of a TDH3 Promoter-deficient Mutant

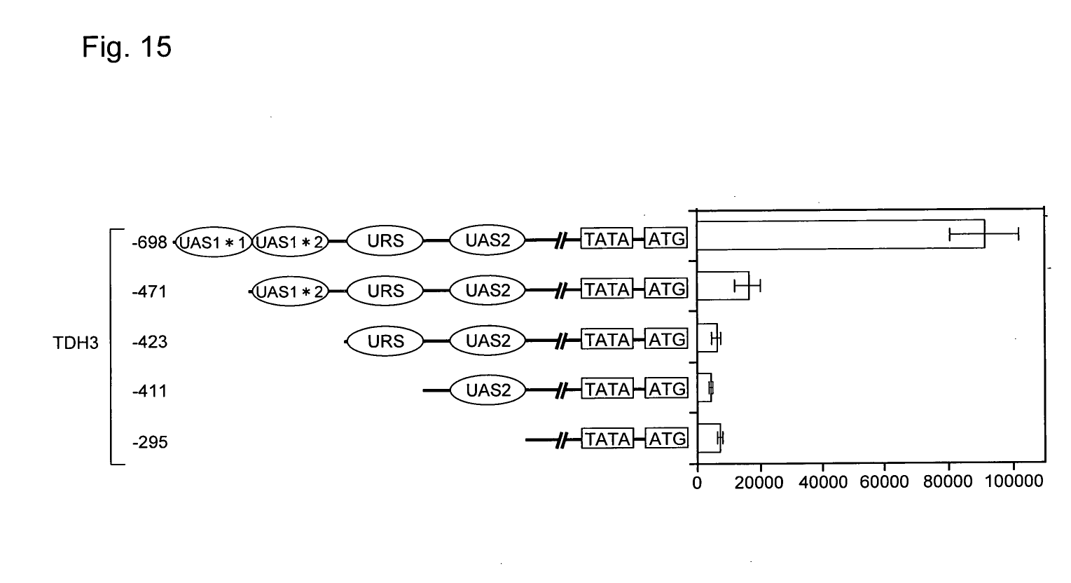

A promoter of the TDH3 gene (systematic gene name: YGR192C) of Saccharomyces cerevisiae has a nucleotide sequence that contains a plurality of control sequences, which are important for regulation of expression (see JBC, 1994, 269: 6153-6162, and FIG. 15). Thus, it was examined whether or not it would be possible to detect the promoter activity corresponding to removal of each control sequence using mCLuc after the removal of the sequence.

1-1. Production of a Plasmid in Which a TDH3 Promoter (−471) is Linked to the 5′ Upstream Region of mCLuc DNA

The TDH3 promoter (−471) has a promoter sequence designed for examining how the promoter activity changes with the removal of a fermentable carbon source-dependent upstream activation sequence (UAS1*1) that exists between −486 bp and −474 bp 5′ upstream from the initiation codon of TDH3 ORF.

The TDH3 promoter (−471) was obtained by PCR using the primer described below and the 3′TDH3 primer (SEQ ID NO: 24).

(SEQ ID NO: 90)

TDH3-471_F: AAGAGGATCCAATGGAGCCCGCTTTTTAAG

The TDH3-471_F primer has a sequence in which a BamHI restriction enzyme site is added to the 5′ region of 20 bp 3′ downstream starting from 471 bp 5′ upstream of TDH3 ORF. In addition, see the Yeast Genome Database (http://www.yeastgenome.org/) regarding the position of the primer.

PCR was carried out under basically the same conditions used for the above mature CLuc cDNA synthesis (Example 1) except that: Saccharomyces cerevisiae S288C genomic DNA (1 ng) was used as a template; TDH3-471_F (SEQ ID NO: 90) and 3′TDH3 (SEQ ID NO: 24) were used as primers; the annealing temperature was 54° C. and the elongation reaction time was 1 minute in a second step; and a third step was carried out for 2 minutes.

The obtained PCR product was analyzed by 2% agarose gel electrophoresis such that a DNA fragment (of approximately 470 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The DNA fragment is referred to as the “TDH3 promoter (−471) DNA fragment.”

The TDH3 promoter (−471) DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the TDH3 promoter (−471) had been inserted was identified. A plasmid (hereafter to be referred to as “pmCLuRA-TDH3 (−471)”) was prepared from the transformant retaining a plasmid into which DNA encoding the TDH3 promoter (−471) had been inserted.

1-2. Production of the Plasmid in Which a TDH3 Promoter (−423) is Linked to the 5′ Upstream Region of mCLuc DNA

The TDH3 promoter (−423) has a promoter sequence designed for examining how the promoter activity changes with the removal of the UAS 1* 1 described above and a fermentable carbon source-dependent upstream activation sequence (UAS1*2) that exists between −448 bp and −436 bp 5′ upstream from the initiation codon of TDH3 ORF.

The TDH3 promoter (−423) was obtained by PCR using a primer described below and the 3′TDH3 primer (SEQ ID NO: 24).

(SEQ ID NO: 91)

TDH3-423_F: AAGAGGATCCAGAATCCCAGCACCAAAATA

The TDH3-423_F primer has a sequence in which a BamHI restriction enzyme site is added to the 5′region of 20 bp 3′ downstream starting from 423 bp 5′ upstream of TDH3 ORF. In addition, see the Yeast Genome Database (http://www.yeastgenome.org/) regarding the position of the primer.

PCR was carried out under basically the same conditions used for the above mature CLuc cDNA synthesis (Example 1) except that: Saccharomyces cerevisiae S288C genomic DNA (1 ng) was used as a template; TDH3-423_F (SEQ ID NO: 91) and 3′TDH3 (SEQ ID NO: 24) were used as primers; the annealing temperature was 54° C. and the elongation reaction time was 1 minute in a second step; and a third step was carried out for 2 minutes.

The obtained PCR product was analyzed by 2% agarose gel electrophoresis such that a DNA fragment (of approximately 420 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The DNA fragment is referred to as “TDH3 promoter (−423) DNA fragment.”

The TDH3 promoter (−423) DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the TDH3 promoter (−423) had been inserted was identified. A plasmid (hereafter to be referred to as “pmCLuRA-TDH3 (−423)”) was prepared from the transformant retaining a plasmid into which DNA encoding the TDH3 promoter (−423) had been inserted.

1-3. Production of the Plasmid in Which a TDH3 Promoter (−411) is Linked to the 5′ Upstream Region of mCLuc DNA

The TDH3 promoter (−411) has a promoter sequence designed for examining how the promoter activity changes with the removal of the UAS1*1 and UAS1*2 described above and a fermentable carbon source-dependent upstream repression sequence (URS) that exists between −431 bp and −419 bp 5′ upstream from the initiation codon of TDH3 ORF.

The TDH3 promoter (−411) was obtained by PCR using a primer described below and the 3′TDH3 primer (SEQ ID NO: 24).

(SEQ ID NO: 92)

TDH3-411_F: AAGAGGATCCTGTTTTCTTCACCAACCATC

The TDH3-411 1F primer has a sequence in which a BamHI restriction enzyme site is added to the 5′ region of 20 bp 3′ downstream starting from 411 bp 5′ upstream of TDH3 ORF. In addition, see the Yeast Genome Database (http://www.yeastgenome.org/) regarding the position of the primer.

PCR was carried out under basically the same conditions used for the above mature CLuc cDNA synthesis (Example 1) except that: Saccharomyces cerevisiae S288C genomic DNA (1 ng) was used as a template; TDH3-411_F (SEQ ID NO: 92) and 3′TDH3 (SEQ ID NO: 24) were used as primers; the annealing temperature was 54° C. and the elongation reaction time was 1 minute in a second step; and a third step was carried out for 2 minutes.

The obtained PCR product was analyzed by 2% agarose gel electrophoresis such that a DNA fragment (of approximately 410 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The DNA fragment is referred to as “TDH3 promoter (−411) DNA fragment.”

The TDH3 promoter (−411) DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the TDH3 promoter (−411) had been inserted was identified. A plasmid (hereafter to be referred to as “pmCLuRA-TDH3 (−411)”) was prepared from the transformant retaining a plasmid into which DNA encoding the TDH3 promoter (−411) had been inserted.

1-4. Production of the Plasmid in Which a TDH3 Promoter (−295) is Linked to the 5′ Upstream Region of mCLuc DNA

The TDH3 promoter (−295) has a promoter sequence designed for examining how the promoter activity changes with the removal of the UAS1*1, UAS1*2, and URS described above and a non-fermentable carbon source-dependent upstream activation sequence (UAS2) that exists between −305 bp and −297 bp 5′ upstream from the initiation codon of TDH3 ORF.

The TDH3 promoter (−295) was obtained by PCR using a primer described below and the 3′TDH3 primer (SEQ ID NO: 24).

(SEQ ID NO: 93)

TDH3-295_F: AAGAGGATCCGGAGTAAATGATGACACAAG

The TDH3-295_F primer has a sequence in which a BamHI restriction enzyme site is added to the 5′ region of 20 bp 3′ downstream starting from 295 bp 5′ upstream of TDH3 ORF. In addition, see the Yeast Genome Database (http://www.yeastgenome.org/) regarding the position of the primer.

PCR was carried out under basically the same conditions used for the above mature CLuc cDNA synthesis (Example 1) except that: Saccharomyces cerevisiae S288C genomic DNA (1 ng) was used as a template; TDH3-295_F (SEQ ID NO: 93) and 3′TDH3 (SEQ ID NO: 24) were used as primers; the annealing temperature was 54° C. and the elongation reaction time was 1 minute in a second step; and a third step was carried out for 2 minutes.

The obtained PCR product was analyzed by 2% agarose gel electrophoresis such that a DNA fragment (of approximately 290 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The DNA fragment is referred to as “TDH3 promoter (−295) DNA fragment.”

The TDH3 promoter (−295) DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the TDH3 promoter (−295) had been inserted was identified. A plasmid (hereafter to be referred to as pmCLuRA-TDH3 (−295)”) was prepared from the transformant retaining a plasmid into which DNA encoding the TDH3 promoter (−295) had been inserted.

(2) Transformation of Yeast (Saccharomyces cerevisiae Strain YPH500) With the Use of Reporter Plasmids Comprising Each Deficient Mutant TDH3 Promoter

The Saccharomyces cerevisiae strain YPH500 was transformed with the use of the following 5 plasmids: pmCLuRA-TDH3 (−471), pmCLuRA-TDH3(−423), pmCLuRA-TDH3(−41 1), pmCLuRA-TDH3(−295), and pmCLuRA-TDH3 (corresponding to “−698” in FIG. 15). An EZ-transformation kit (BIO101) was used for transformation.

The obtained transformant was applied to an uracil-free synthetic agar medium (SD+KHLadeW), followed by culture at 30° C. for 3 days.

After culture for 3 days, transformants each comprising a different plasmid were obtained.

(3) Measurement of the Activity of Each Mutant TDH3 Promoter

Three colonies each of a different single clone of a transformant comprising a plasmid obtained in (2) above were introduced into a synthetic liquid medium (SD+KHLadeW 200 mM KPi), followed by overnight culture. Then, 5 μl of the obtained culture solution that had reached the stationary phase after overnight culture was introduced into each well of a 96-deep well plate, with 1 ml of the same medium having been added to such well. This was followed by shake culture at 30° C. 16 hours after the initiation of culture, a portion of the culture solution was recovered, followed by measurement of absorbance at 600 nm (OD600) and relative light units (RLU) from a luminometer in accordance with the method in Example 1. The activity of each promoter was digitized based on the obtained values as with the case of Example 11. FIG. 15 shows the results.

(4) Production of a GAL1 Promoter-deficient Mutant

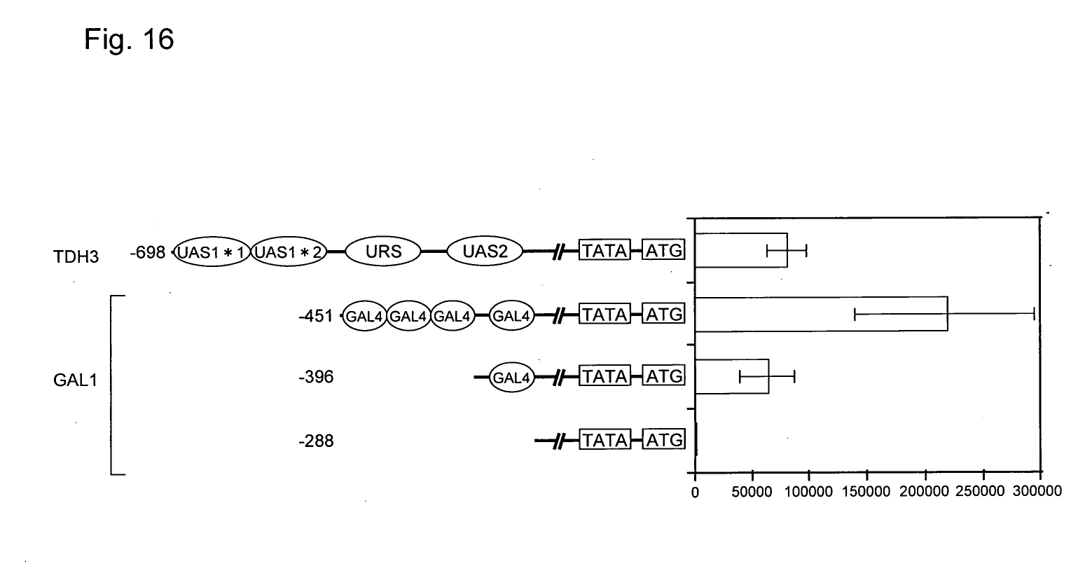

A promoter of the GAL1 gene (systematic gene name: YBR020W) of Saccharomyces cerevisiae has a nucleotide sequence that contains four GAL4 binding domains (corresponding to “GAL4” in FIG. 16), which are important for regulation of expression (see: Giniger, E. et al., Cell, 40: 767-774 (1985); Johnston and Davis, Mol. Cell. Biol. 4: 1440-1448 (1984); Yocum, R. R. et al., Mol. Cell. Biol. 4: 1985-1998 (1984); and FIG. 16). Thus, it was examined whether or not it would be possible to detect the promoter activity corresponding to removal of these sequences using mCLuc after the removal of the sequence.

4-1. Production of a Plasmid in Which a GAL1 Promoter (−396) is Linked to the 5′ Upstream Region of mCLuc DNA

The GAL1 promoter (−396) has a promoter sequence designed for examining how the promoter activity changes with the removal of the GAL4 binding domains (3 repetitions of GAL4), which are located between −451 bp and −397 bp 5′ upstream from the initiation codon of GAL1 ORF.

The GAL1 promoter (−396) was obtained by PCR using a primer described below and the 3′GAL1 primer (SEQ ID NO: 78).

(SEQ ID NO: 94)

GAL1-396_F: AAGAGGATCCTGCGTCCTCGTCTTCACCGG

The GAL1-396_F primer has a sequence in which a BamHI restriction enzyme site is added to the 5′ region of 20 bp 3′ downstream starting from 396 bp 5′ upstream from the initiation codon of GAL1 ORF. In addition, see the Yeast Genome Database (http:/Hwww.yeastgenome.org/) regarding the position of the primer.

PCR was carried out under basically the same conditions used for the above mature CLuc cDNA synthesis (Example 1) except that: Saccharomyces cerevisiae S288C genomic DNA (1 ng) was used as a template; GAL1-396_F (SEQ ID NO: 94) and 3′GAL1 (SEQ ID NO: 78) were used as primers; the annealing temperature was 54° C. and the elongation reaction time was 1 minute in a second step; and a third step was carried out for 2 minutes.

The obtained PCR product was analyzed by 2% agarose gel electrophoresis such that a DNA fragment (of approximately 390 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The DNA fragment is referred to as “GAL1 promoter (−396) DNA fragment.”

The GAL1 promoter (−396) DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the GAL1 promoter (−396) had been inserted was identified. A plasmid (hereafter to be referred to as “pmCLuRA-GAL1 (−396)”) was prepared from the transformant retaining a plasmid into which DNA encoding the GAL1 promoter (−396) had been inserted.

4-2. Production of a Plasmid in Which a GAL1 Promoter (−288) is Linked to the 5′ Upstream Region of mCLuc DNA

The GAL1 promoter (−288) has a promoter sequence designed for examining how the promoter activity changes with the removal of the aforementioned GAL4 binding domains, which are 3 repetitions of GAL4, and the GAL4 binding domain that independently exists between −349 bp and −332 bp 5′ upstream from the initiation codon of GAL1 ORF.

The GAL1 promoter (−288) was obtained by PCR using a primer described below and the 3′GAL1 primer (SEQ ID NO: 78).

(SEQ ID NO: 95)

GAL1-288_F: AAGAGGATCCGAAAAATTGGCAGTAACCTG

The GAL1-288_F primer has a sequence in which a BamHI restriction enzyme site is added to the 5′ region of 20 bp 3′ downstream starting from 288 bp 5′ upstream from the initiation codon of GAL1 ORF. In addition, see the Yeast Genome Database (http://www.yeastgenome.org/) regarding the position of the primer.

PCR was carried out under basically the same conditions used for the above mature CLuc cDNA synthesis (Example 1) except that: Saccharomyces cerevisiae S288C genomic DNA (1 ng) was used as a template; GAL1-288_F (SEQ ID NO: 95) and 3′GAL1 (SEQ ID NO: 78) were used as primers; the annealing temperature was 54° C. and the elongation reaction time was 1 minute in a second step; and a third step was carried out for 2 minutes.

The obtained PCR product was analyzed by 2% agarose gel electrophoresis such that a DNA fragment (of approximately 390 bp) was confirmed. The obtained PCR product was purified using a GenElute PCR clean-up kit. Then, DNA was eluted from the column of the kit with 40 μl of distilled water. The obtained DNA solution was subjected to cleavage with BamHI (50 U) in 50 μl of a reaction solution for 18 hours, followed by purification using a GenElute PCR clean-up kit. Next, DNA was eluted from the column of the kit with 40 μl of distilled water. The DNA fragment is referred to as “GAL1 promoter (−288) DNA fragment.”

The GAL1 promoter (−288) DNA fragment and the pmCLuRA Bam HI-Sma I fragment were subjected to ligation and circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid into which DNA encoding the GAL1 promoter (−288) had been inserted was identified. A plasmid (hereafter to be referred to as “pmCLuRA-GAL1(−288)”) was prepared from the transformant retaining a plasmid into which DNA encoding the GAL1 promoter (−288) had been inserted.

(5) Transformation of Yeast (Saccharomyces cerevisiae Strain YPH500) With the Use of Each Reporter Plasmid Comprising a Deficient Mutant GAL1 Promoter

Saccharomyces cerevisiae strain YPH500 was transformed with the use of the following 3 plasmids: pmCLuRA-GAL1 (−396); pmCLuRA-GAL1 (−288); and pmCLuRA-GAL1 (corresponding to “−451” in FIG. 16). An EZ-transformation kit (BIO101) was used for transformation.

The obtained transformant was applied to an uracil-free synthetic agar medium (SD+KHLadeW), followed by culture at 30° C. for 3 days.

After culture for 3 days, transformants each comprising a different plasmid were obtained.

(6) Measurement of the Activity of Each Deficient Mutant GAL1 Promoter

Three colonies each of a different single clone of a transformant comprising a plasmid obtained in (5) above were introduced into a synthetic liquid medium (SD+KHLadeW 200 mM KPi), followed by overnight culture. Then, 20 μl of the obtained culture solution that had reached the stationary phase after overnight culture was introduced into each well of a 96-deep well plate, with 1 ml of a 2% galactose-containing synthetic liquid medium (SC+KHLadeW 200 mM KPi) having been added to such well. This was followed by shake culture at 30° C. 32 hours after the initiation of culture, a portion of the culture solution was recovered, followed by measurement of absorbance at 600 nm (OD600) and relative light units (RLU) from a luminometer in accordance with the method in Example 1. The activity of each promoter was digitized based on the obtained values as with the case of Example 11. FIG. 16 shows the results. Also, FIG. 16 shows the results of measurement of CLuc activity using a culture solution of a transformant comprising DNA encoding mCLuc linked to the TDH3 promoter as comparison.

(7) Result of Search for a Cis Sequence Involved in Transcriptional Activation Within Promoters

As shown in FIGS. 15 and 16, the values measured using mCLuc as a reporter enzyme changed significantly based on the presence or absence of a cis sequence that is considered to be involved in transcriptional activation within the TDH3 promoter and the GAL1 promoter. The correlation between the promoter fragments and measurement values of mCLuc reporter assay corresponds well to the results described in JBC, 1994, 269: 6153-6162. Based on such results, it has been found that the method using mCLuc as a reporter enzyme can be used for experimentation for examination of a sequence involved in transcriptional activation by sequentially shortening promoters similar to those used in conventional methods.

### Example 15

### Establishment of a Method for Examining Gene Mutation Using CLuc

When mutation suddenly occurs in a gene, it sometimes results in a situation whereby a codon that originally encodes an amino acid at the mutation site or in the 3′ downstream region in cases of insertion or deletion of a base becomes the termination codon (Such mutation is referred to as “nonsense mutation”). Accordingly, a portion of a protein may be produced, instead of the entire length of the protein that is usually produced. Many cases are known in which such portion of a protein causes diseases. This is because, in many cases, a portion of a protein alone cannot sufficiently function as the original protein. A method using CLuc with improved convenience and measurement sensitivity has been established as a method for examining genetic abnormalities such as nonsense mutations that prevent normal protein translation in various types of animal individuals, such as humans.

Specifically, a method for detecting nonsense mutations by reporter assay using CLuc has been developed, such mutation having occurred in the third exon (a nucleotide sequence between the 2567^{th} and the 3402^{nd} bases of the nucleotide sequence set forth in SEQ ID NO: 96) in the rat Apo E gene (SEQ ID NO: 96: J. Biol. Chem. 261, 13777-13783 (1986)).

The nucleotide sequence set forth in SEQ ID NO: 97 is a sequence comprising the coding region of the third exon of the rat Apo E gene and the termination codon at the 3′ end thereof.

In the experiments described below, DNA encoding the total amino acid sequence of the coding region contained in the third exon of the rat Apo E gene (a nucleotide sequence between the 1^{st} and the 723^{rd} bases of the nucleotide sequence set forth in SEQ ID NO: 97; hereafter to be referred to as “ApoE(+)”) and DNA obtained by adding the termination codon to ApoE(+) (a nucleotide sequence between the 1^{st} and the 726^{th} bases of the nucleotide sequence set forth in SEQ ID NO: 97; hereafter to be referred to as “ApoE(−)”) were used as a normal DNA model and a nonsense mutation model DNA, respectively. Then, three cases were identified in which: both ApoE genes on two homologous chromosomes were ApoE(+); both ApoE genes on two homologous chromosomes were ApoE(−); and one of the ApoE genes on two homologous chromosomes was ApoE(+) while the other was ApoE(−).

(1) Construction of the Plasmid pCLuTr

In order to establish a plasmid vector used for examining genetic abnormality, a restriction enzyme site was introduced between α-factor secretory signal peptide DNA and mature CLuc DNA of DNA encoding αCLuc in the plasmid pCLuRA produced in Example 1. Then, PCR was carried out using the primers described below.

aCL_inv_5′-Hind-Sph:

(SEQ ID NO: 98)

CCCTAAGCTTATCGCATGCCAGGACTGTCCTTACGAACCTGATCCAC
aCL_inv_3′-Hind:

(SEQ ID NO: 99)

GGGTAAGCTTGAGCTTCAGCCTCTCTTTTCTCGAGAG

The αCL_inv_{—}5′-Hind-Sph primer has a sequence in which HindIII and SphI restriction enzyme sites are added to the 5′ region of a 28-bp sequence starting from the 268^{th} base (the 1^{st} bp of the first codon of mature CLuc) of DNA encoding αCLuc (SEQ ID NO: 13). The αCL_inv_{—}3′-Hind primer has a sequence complementary to a 26-bp sequence (26 bp from the 3′ end region of α-factor secretory signal peptide DNA (SEQ ID NO: 11)) starting from the 242^{nd} base of DNA encoding α-CLuc (SEQ ID NO: 13), which has a single base “G” and a HindIII restriction enzyme site added to the 5′ region thereof. Such single base “G” is located in a manner such that homologous recombination with a foreign DNA fragment (ApoE(+) or ApoE(−) in the cases described below) does not occur and frame shift takes place such that CLuc is not expressed when a ring-opened reporter plasmid (the “pCLuTr Hind III-Sph I DNA fragment” in cases described below) is self-circularized during the steps of in vivo recombination described below. When homologous recombination with a foreign DNA fragment occurs during the steps of in vivo recombination, such single base “G” does not remain in a plasmid, resulting in the expression of a fusion protein of CLuc.

PCR was carried out under basically the same conditions used for the above mature CLuc cDNA synthesis (Example 1) except that: αCL_inv_{—}5′-Hind-Sph primer (SEQ ID NO: 98) and αCL_inv_{—}3′-Hind primer (SEQ ID NO: 99) were used; 5% DMSO was added to a reaction solution; and 10 ng of a pCLuRA-TDH3 plasmid was used as a template in the following steps: a first step at 94° C. for 2 minutes; a second step at 94° C. for 15 seconds (denaturation), 52° C. for 30 seconds (annealing), and 68° C. for 8 minutes (elongation) for 35 cycles; and a third step at 68° C. for 10 minutes.

The obtained PCR product was analyzed by 1% agarose gel electrophoresis such that a DNA fragment (of approximately 7 kbp) was confirmed. The obtained PCR product was purified using a GenElute PCR clean-up kit. Then, DNA was eluted from the column of the kit with 40 μl of distilled water. The obtained DNA solution was subjected to cleavage with HindIII (50 U) in 50 μl of a reaction solution for 18 hours, followed by purification using a GenElute PCR clean-up kit. Next, DNA was eluted from the column of the kit with 40 μl of distilled water. The DNA fragment is referred to as the “pCLuTr DNA fragment.”

Subsequently, the pCLuTr DNA fragment was subjected to ligation and self-circularization using a DNA Ligation kit. The resultant was introduced into Escherichia coli DH5α. The obtained transformant was cultured overnight, followed by extraction of a plasmid using a GenElute plasmid MiniPrep kit. In addition, the extracted plasmid was subjected to analysis in terms of the restriction enzyme cleavage pattern and the nucleotide sequence. Thus, a transformant retaining a plasmid in which the pCLuTr DNA fragment had been circularized (hereafter to be referred to as “pCLuTr”) was identified. pCLuTr was prepared using the transformant retaining pCLuTr.

A portion of the obtained pCLuTr (5 μg) was cleaved with HindIII (50 U) in 50 μl of a reaction solution for 18 hours. After cleavage with HindIII, the resultant was purified using a GenElute PCR clean-up kit. Then, DNA was eluted from the column of the kit with 40 μl of distilled water. Thereafter, DNA was cleaved with SphI (50 U) in 50 μl of a reaction solution for 18 hours, followed by purification using a GenElute PCR clean-up kit. Next, DNA was eluted from the column of the kit with 40 μl of distilled water. The entire eluate was subjected to 1% agarose gel electrophoresis. A band (of approximately 7 kbp) was cleaved out therefrom, followed by extraction of a DNA fragment by a gel extraction method using phenol/chloroform. The DNA fragment is referred to as “pCLuTr Hind III-Sph I DNA fragment.”

(2) Preparation of Model DNA of the Coding Region That Exists in the Third Exon of the Rat ApoE Gene

With the use of in vivo recombination of yeasts, in order to incorporate a coding region that exists in the third exon of the rat ApoE gene into a pCLuTr Hind III-Sph I DNA fragment, a coding region that exists in the third exon of the rat ApoE gene having sequences complementary to both ends (of approximately 40 bp) of a pCLuTr Hind III-Sph I DNA fragment was prepared by PCR using primers described below.

ApoE-aCL_gapF:

(SEQ ID NO: 100)

GAAGAAGGGGTATCTCTCGAGAAAAGAGAGGCTGAAGCTGTACTGATGGA
GGACACTATG
ApoE-aCL_gapR:

(SEQ ID NO: 101)

GAACTGTGTTTGGTGGATCAGGTTCGTAAGGACAGTCCTGTTGATTCTCC
AGGGGCACTG

The ApoE-αCL_gapF primer has a sequence in which a 21-bp sequence starting from the 1^{st} base of the coding region (SEQ ID NO: 97) contained in the third exon of the rat ApoE gene is added to the 3′ region of a 39-bp sequence (39 bp from the 3′ end region of α-factor secretory signal peptide DNA (SEQ ID NO: 11)) starting from the 229^{th} base of DNA encoding (αCLuc (SEQ ID NO: 13). The ApoE-αCL_gapR primer has a sequence in which a sequence complementary to a nucleotide sequence between the 704^{th} to the 723^{rd} bases of the coding region (SEQ ID NO: 97) contained in the third exon of the rat Apo E gene is added to the 3′ region of a sequence complementary to a 40-bp sequence 3′ downstream of a position located 268 bp from the initiation codon of αCLuc ORF (the 1^{st} bp of the first codon of mature CLuc).

PCR was carried out under basically the same conditions used for the above mature CLuc cDNA synthesis (Example 1) except that: an ApoE-αCL_gapF primer (SEQ ID NO: 100) and an ApoE-aCL_gapR primer (SEQ ID NO: 101) were used; 5% DMSO was added to a reaction solution; and 10 ng of rat genomic DNA was used as a template in the following steps: a first step at 94° C. for 2 minutes; a second step at 94° C. for 15 seconds (denaturation), 50° C. for 30 seconds (annealing), and 68° C. for 1 minute (elongation) for 35 cycles; and a third step at 68° C. for 2 minutes.

The obtained PCR product was analyzed by 1% agarose gel electrophoresis such that a DNA fragment (of approximately 800 bp) was confirmed. The obtained PCR product was purified using a GenElute PCR clean-up kit. Then, DNA was eluted from the column of the kit with 40 μl of distilled water. The DNA fragment is referred to as the “ApoE(+).”

Subsequently, a coding region (comprising the termination codon) that exists in the third exon of the rat ApoE gene having sequences complementary to both ends (of approximately 40 bp) of a pCLuTr Hind III-Sph I DNA fragment was prepared by PCR using the ApoE-aCL_gapF primer (SEQ ID NO: 100) and a primer described below.

ApoE-aCL_gapR_NC:

(SEQ ID NO: 102)

GAACTGTGTTTGGTGGATCAGGTTCGTAAGGACAGTCCTGTCATTGATTC
TCCAGGGGCACTG

The ApoE-aCL_gapR_NC primer has a sequence in which a sequence complementary to a nucleotide sequence between the 704^{th} and the 726^{th} bases of the coding region (SEQ ID NO: 97) contained in the third exon of the rat ApoE gene is added to the 3′ region of a sequence complementary to a 40-bp sequence 3′ downstream from a position that is 268 bp from the initiation codon of αCLuc ORF.

PCR was carried out as with the case of PCR regarding the ApoE(+) described above except that the ApoE-aCL_gapF primer (SEQ ID NO: 100) and the ApoE-αCL_gapR_NC primer (SEQ ID NO: 102) were used.

The obtained PCR product was analyzed by 1% agarose gel electrophoresis such that a DNA fragment (of approximately 800 bp) was confirmed. The obtained PCR product was purified using a GenElute PCR clean-up kit. Then, DNA was eluted from the column of the kit with 40 μl of distilled water. The DNA fragment is referred to as “ApoE(−).”

Subsequently, solutions containing the obtained DNA fragments of Apo E(+) and Apo E(−), respectively, were serial-diluted, followed by 1% agarose gel electrophoresis. Band intensities were digitized based on the obtained electrophoresis images. Then, dilution was carried in a manner such that the concentration of the DNA fragment of Apo E(+) (not comprising the termination codon) became equivalent to that of the DNA fragment of Apo E(−) (comprising the termination codon). OD 260 of each obtained diluted DNA fragment was measured, followed by quantification of DNA concentration. Further, equal amounts of the DNA fragments of Apo E(+) and Apo E(−) were mixed together such that Apo E(±) (where the ratio between Apo E(+) and Apo E(−) was 1:1) was produced.

(3) Establishment of a Method for Examining Genetic Abnormalities Using CLuc

A single colony of Saccharomyces cerevisiae strain YPH500 was introduced into a 2-fold concentrated YPD liquid medium, followed by overnight shake culture at 30° C. Further, 10 μl of the culture solution was introduced into 100 ml of a YPD medium, followed by shake culture at 30° C. When the OD600 value reached 0.6, culture was discontinued. Cells were recovered therefrom so as to be agitated again in distilled water. Then, cells are recovered again. The obtained cells were suspended in 1 ml of LiAc-TE Buffer (100 mM Tris-HCl/10 mM EDTA (pH 8.0): 1M Li acetate: water=1:1:8) that had been prepared immediately before use using a pipette. The resultants were determined to be yeast competent cells.

A pCLuTr Hind III-SphI DNA fragment (3 μg) and 100 μl of the herring sperm DNA (Clontech) were added to 1 ml of the competent cells, followed by sufficient mixing. 10 μl of the resultant was dispensed into each well of a 96-well PCR tube. In addition, 1 μl of the above ApoE(+), ApoE(−), or ApoE(±) solution that had been adjusted to 10-50 ng/μl was added thereto. Further, as a negative control, a sample not containing any of the DNA solutions was used. 60 μl of a PEG solution (100 mM Tris-HCl/10 mM EDTA (pH 8.0): 1M Li acetate: 50% Polyethylene glycol 4000=1:1:8) that had been prepared immediately before use was dispensed into each well of a 96-well PCR tube, followed by sufficient mixing. After incubation at 30° C. for 30 minutes, 7 μl of DMSO was added thereto. The resultant was sufficiently mixed, followed by incubation at 42° C. for 15 minutes. Then, the resultant was immediately cooled in ice. 130 μl of a culture solution (SD+WKHLade 200 mM Kpi; pH 6.0) was added to each well containing the obtained solution, followed by sufficient agitation (with the resultant hereafter to be referred to as “transformation mix”). 1 ml of a culture solution (SD+WKHLade 200 mM Kpi; pH 6.0) was added to each well of a 96-deep well plate. The above transformation mix (50 μl) was introduced into each well, followed by culture at 30° C. at 160 r/min for 5 days.

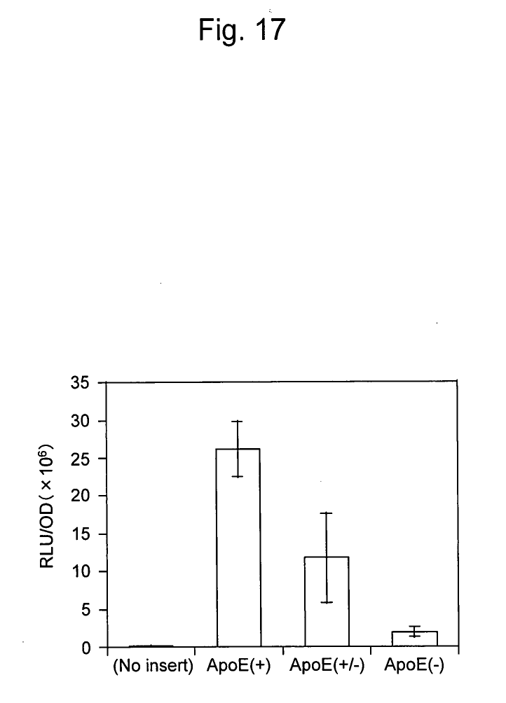

Subsequently, it was confirmed that the stationary phase had been achieved in all wells. Then, 1 ml of a culture solution (SD+WKHLade 200 mM Kpi; pH 6.0) was added to each well of a new 96-deep well plate. The above culture solution (10 μl) was introduced into each well of the plate, followed by culture for approximately 16 hours. A portion of the culture solution was recovered so that absorbance at 600 nm (OD600) and relative light units (RLU) from a luminometer were measured in accordance with the method of Example 1. Based on the obtained values, a value obtained by dividing relative light units (RLU) by absorbance at 600 nm was calculated. FIG. 17 shows the results. In addition, in FIG. 17, the expression “No insert” indicates the result of a negative control.

As shown in FIG. 17, it has been found that CLuc is secreted and has the activity even when CLuc is expressed as a fusion protein of CLuc and a protein encoded by the third exon of the rat ApoE gene. In addition, the CLuc activity corrected with the turbidity of Apo E(−) (sequence comprising the termination codon) is significantly lower than the CLuc activity corrected with the turbidity of Apo E(+) (sequence not comprising the termination codon). In addition, the CLuc activity corrected with the turbidity of Apo E(±) was found to be of a level that was almost half of the level of the activity of Apo E(+). Based on the results, it has been found that gene mutation in the coding region of the third exon of the Apo E gene can be conveniently detected based on reporter assay using CLuc by using, as a starting material, rat genomic DNA regarding which information concerning a genetic abnormality of the third exon in the Apo E gene has not been obtained and comparing a case in which such rat genomic DNA is used with a case in which the above model DNA is used.

In addition, the method for plasmid construction and yeast transformant production using the in vivo recombination of yeast used in the present Example is significantly convenient. Thus, it is considered that many types of DNAs can be quickly incorporated into reporter plasmids during reporter assay using CLuc, even in a case in which any type of DNA (such as a promoter or a gene) is used. Therefore, it has been demonstrated that high-throughput reporter assay using CLuc can be achieved. Moreover, the operations described above basically consist of dispensing operations. Thus, automatization using a dispensing robot (e.g. Beckman Coulter Biomek 200) has already been achieved.

Free Text of Sequence Listing

SEQ ID NO: 13 represents a gene encoding a fusion protein.

SEQ ID NO: 14 represents a fusion protein.

SEQ ID NO: 15 represents a synthetic gene.

SEQ ID NOS: 16 to 19, 23, 24, 53 to 60, 62, 63, 65, 66, 68, 69, 71, 72, 74, 75, 77, 78, 80 to 95, and 98 to 102 represent primers.

SEQ ID NOS: 20 and 21 represent oligo DNAs.

SEQ ID NOS: 25 to 52 represent synthetic DNAs.

SEQ ID NO: 97 represents a sequence comprising the coding region of the third exon of the rat Apo E gene and the termination codon at the 3′ end thereof.

All publications, patents, and patent applications cited herein are incorporated herein by reference in their entirety.

1. A reporter assay method, comprising:
a first step of introducing a gene encoding a secretory luminescent enzyme into a host;
a second step of allowing the culture or culture supernatant of the transformant obtained in the first step to come into contact with the substrate of the secretory luminescent enzyme; and
a third step of measuring the enzyme activity of the secretory luminescent enzyme;
wherein the expression, functions, transcriptional activity, or transcriptional control functions of the foreign gene or foreign DNA fragment that has been introduced into the host is evaluated based on the enzyme activity of the secretory luminescent enzyme.

2. The reporter assay method according to claim 1, wherein the foreign gene is linked to the gene encoding a secretory luminescent enzyme or is inserted between a gene encoding a secretory signal peptide and a gene encoding a mature protein in the gene encoding a secretory luminescent enzyme.

3. The reporter assay method according to claim 1, wherein the foreign gene is expressed in the host.

4. The reporter assay method according to claim 1, wherein the foreign DNA fragment is linked to the gene encoding a secretory luminescent enzyme.

5. The reporter assay method according to claim 1, wherein the secretory luminescent enzyme is a secretory luciferase.

6. The reporter assay method according to claim 5, wherein the secretory luciferase is Cypridina luciferase.

7. The reporter assay method according to claim 6, wherein the Cypridina luciferase is a Cypridina noctiluca-derived luciferase.

8. The reporter assay method according to claim 1, wherein the secretory luminescent enzyme is a fusion protein of a secretory signal peptide that functions in the host and a mature protein of a secretory luminescent enzyme.

9. The reporter assay method according to claim 1, wherein the host is a yeast.

10. The reporter assay method according to claim 9, wherein the yeast is Saccharomyces cerevisiae.

11. The reporter assay method according to claim 9, wherein a transformant of the yeast is cultured under conditions of pH 3.5 to 6.5.

12. A reporter assay method, comprising:
a first step of introducing into a host DNA in which a gene encoding a secretory signal peptide or secretory protein is linked to the upstream of a gene encoding a mature protein of a secretory luminescent enzyme;
a second step of allowing the culture or culture supernatant of the transformant obtained in the first step to come into contact with the substrate of the secretory luminescent enzyme; and
a third step of measuring the enzyme activity of the secretory luminescent enzyme;
wherein the secretion capacity of the secretory signal peptide or secretory protein is evaluated based on the enzyme activity of the secretory luminescent enzyme.

13. The reporter assay method according to claim 12, wherein the secretory luminescent enzyme is a secretory luciferase.

14. The reporter assay method according to claim 13, wherein the secretory luciferase is Cypridina luciferase.

15. The reporter assay method according to claim 14, wherein the Cypridina luciferase is a Cypridina noctiluca-derived luciferase.

16. The reporter assay method according to claim 12, wherein the host is a yeast.

17. The reporter assay method according to claim 16, wherein the yeast is Saccharomyces cerevisiae.

18. The reporter assay method according to claim 16, wherein a transformant of the yeast is cultured under conditions of pH 3.5 to 6.5.

19. A reporter assay method, comprising:
a first step of introducing a gene encoding a secretory luminescent enzyme into a host;
a second step of allowing the culture or culture supernatant of the transformant obtained in the first step to come into contact with the substrate of the secretory luminescent enzyme; and
a third step of measuring the enzyme activity of the secretory luminescent enzyme;
wherein chemical or physical change is measured based on the enzyme activity of the secretory luminescent enzyme.

20. The reporter assay method according to claim 19, wherein the method comprises a step of exposing the transformant to a subject of chemical or physical change after the first step.

21. The reporter assay method according to claim 19, wherein a foreign DNA fragment that is responsive to the chemical or physical change is linked to the gene encoding a secretory luminescent enzyme.

22. The reporter assay method according to claim 19, wherein a foreign DNA fragment that interacts with a protein that is responsive to the chemical or physical change is linked to the gene encoding a secretory luminescent enzyme.

23. The reporter assay method according to claim 22, wherein the protein that is responsive to the chemical or physical change is encoded by a foreign gene.

24. The reporter assay method according to claim 19, wherein the secretory luminescent enzyme is a secretory luciferase.

25. The reporter assay method according to claim 24, wherein the secretory luciferase is Cypridina luciferase.

26. The reporter assay method according to claim 25, wherein the Cypridina luciferase is a Cypridina noctiluca-derived luciferase.

27. The reporter assay method according to claim 19, wherein the host is a yeast.

28. The reporter assay method according to claim 27, wherein the yeast is Saccharomyces cerevisiae.

29. The reporter assay method according to claim 27, wherein a transformant of the yeast is cultured under conditions of pH 3.5 to 6.5.
